(19) United States
(12) Patent Application Publication
(10) Pub. No.: US 2024/0252051 A1
(43) Pub. Date: Aug. 1, 2024

(54) METHOD AND SYSTEM FOR GENERATING DYNAMIC REAL-TIME PREDICTIONS USING HEART RATE VARIABILITY

(71) Applicant: SPORTS DATA LABS, INC., Royal Oak, MI (US)

(72) Inventors: Vivek KHARE, Cupertino, CA (US); Anuroop YADAV, Livingston, NJ (US); Mark GORSKI, Royal Oak, MI (US); Stan MIMOTO, Bethel Island, CA (US)

(73) Assignee: SPORTS DATA LABS, INC., Royal Oak, MI (US)

(21) Appl. No.: 18/564,184

(22) PCT Filed: May 25, 2022
(86) PCT No.: PCT/US22/30939

§ 371 Date: Nov. 27, 2023

Related U.S. Application Data
(60) Provisional application No. 63/192,815, filed on May 25, 2021.
(60) Provisional application No. 63/281,950, filed on Nov. 22, 2021.

(51) Int. Cl. A61B 5/024; A61B 5/00; A61B 5/0245; A61B 5/35; A61B 5/352; G16H 40/67

(52) U.S. Cl. CPC A61B 5/02405; A61B 5/0006; A61B 5/0022; A61B 5/0245; A61B 5/35; A61B 5/352; G16H 40/67; A61B 2503/10; A61B 2503/40

(57) ABSTRACT

A method for generating dynamic real-time predictions using heart rate variability is provided. The method includes steps of gathering or calculating R-R intervals derived from one or more source sensors. A primary insight and a reference insight are calculated from the R-R intervals. The primary insight is compared with the reference insights to a predictive indicator. A system implementing the method is also provided.

### CROSS-REFERENCE TO RELATED APPLICATIONS

This application claims the benefit of U.S. provisional application Ser. No. 63/281,950 filed Nov. 22, 2021 and U.S. provisional application Ser. No. 63/192,815 filed May 25, 2021, the disclosures of which are hereby incorporated in their entirety by reference herein.

### TECHNICAL FIELD

In at least one aspect, the present invention is related to methods and systems for generating dynamic real-time predictions using heart rate variability.

### BACKGROUND

Heart rate variability (HRV) is a measure of the variation in time between each heartbeat. HRV is controlled by the autonomic nervous system, which has two divisions—the sympathetic nervous system and the parasympathetic nervous system—that work together to regulate a variety of biological processes and ensure an effective bodily response to a given event. These bodily responses can affect outcomes of events. While HRV can be measured in a variety of ways, it is not traditionally used as a real-time measurement of performance, nor is it used as a real-time indicator of event outcomes. In the context of sports, HRV is not captured in live competition for use as a real-time or near real-time measurement to predict event outcomes.

Accordingly, there is a need for methods and systems that can capture real-time or near real-time HRV and other contextual data in order to predict outcomes of events.

### SUMMARY

In at least one aspect, a method for generating dynamic real-time predictions using heart rate variability is provided. The method includes a step of gathering or calculating subsequent R-R intervals derived from one or more source sensors from a targeted individual where the subsequent R-R intervals are R-R intervals during an event. Differences in subsequent successive R-R intervals are calculated. One or more subsequent heart rate variability values are calculated from the successive differences between heartbeats. Characteristically, one value of the one or more subsequent heart rate variability values is calculated for each sub-event amongst two or more sub-events that comprise at least a portion of the event. HRV difference(s) are calculated based upon the difference between the heart rate variability values for consecutive sub-events and a heart rate variability baseline, divided by the heart rate variability baseline. The difference between two successive HRV differences is calculated to create at least one variability indicator. A threshold is calculated utilizing at least a portion of contextual data to characterize information derived from one or more variability indicators. At least one primary insight is created by comparing the threshold and the at least one variability indicator. The method also includes steps of accessing one or more reference insights and comparing the at least one primary insight and the one or more reference insights to create one or more predictive indicators. In a refinement, the heart rate variability baseline is determined by gathering or calculating R-R intervals derived from the one or more source sensors from a targeted individual prior to the event; calculating differences in successive R-R intervals; calculating one or more heart rate variability values from successive differences between heartbeats; and establishing the heart rate variability baseline for the targeted individual using at least a portion of the calculated one or more heart rate variability values for an event associated with the targeted individual with a definable, quantifiable, measurable, or observable outcome. Advantageously, the heart rate variability baseline can be created, modified, or enhanced based on values collected prior to a start of the event.

In another aspect, a method for generating dynamic real-time predictions using heart rate variability is described. The method includes a step of gathering or calculating R-R intervals derived from one or more source sensors from a targeted individual. The difference in successive R-R intervals is calculated. One or more heart rate variability values are calculated from successive differences between heartbeats (e.g., normal heartbeats and e.g., using at least a portion of an RMSSD-based methodology or formula). A normal heartbeat can be the heartbeat of a subject at rest or prior to an event being analyzed. A normal heartbeat can be the heartbeat of a subject during warmup for an event or the heartbeat during a prior event. A heart rate variability baseline is established for the targeted individual using at least a portion of the calculated one or more heart rate variability values for an event with a definable, quantifiable, measurable, or observable outcome, wherein the heart rate variability baseline is created, modified, or enhanced based upon values collected prior to the start of the event. Subsequent R-R intervals derived from the one or more source sensors from the targeted individual are gathered or calculated. The difference in subsequent successive R-R intervals is calculated. One or more subsequent heart rate variability values are calculated from successive differences between heartbeats (e.g., normal heartbeats and e.g., using at least a portion of an RMSSD-based methodology or formula). One value of the one or more subsequent heart rate variability values is calculated for each sub-event (e.g., one or more events within the event that have a definable outcome, such as a point within a game, with each sub-event having a start time and end time) amongst two or more sub-events that comprise at least a portion of the event. A HRV difference is calculated based upon the difference between the heart rate variability values for consecutive sub-events and the heart rate variability baseline. The HRV difference is divided by the heart rate variability baseline. The difference between two successive HRV Differences is calculated to create at least one variability indicator. A plurality of variability indicators includes the variability indicator. A threshold is created utilizing at least a portion of contextual data to characterize (e.g., categorize, illustrate, characterize) information derived from one or more variability indicators. At least one primary insight is created based upon the comparison of the threshold and at least one variability indicator. One or more reference insights are accessed (e.g., accessed or created). Lastly, the at least one primary insight and the one or more reference insights are compared to create one or more predictive indicators. The one or more steps are executed on one or more computing devices, one or more sensors, or a combination thereof. In a refinement, the at least one primary insight, the one or more reference insights, or the one or more predictive indicators are modified or enhanced based upon new R-R intervals or contextual data being calculated or gathered (e.g., collected or received by one or more computing devices). One or more Artificial Intelligence techniques (e.g., including models, methods, and the like) can be utilized for the one or more creations, modifications, or enhancements.

In another aspect, a system for generating dynamic real-time predictions using heart rate variability is described. The system includes one or more source sensors that gather animal data from a targeted individual wherein at least a portion of the gathered animal data is heart rate variability data (e.g., wherein the HRV data is derived from the animal data or provided as part of the animal data). Animal data is transmitted by the one or more source sensors electronically. A transmission subsystem provides the transmitted animal data to a computing subsystem. The computing subsystem gathers the animal data in real-time or near real-time. The computing subsystem further gathers contextual data related to the gathered animal data and the event associated with the targeted individual. The computing subsystem takes one or more actions with the gathered contextual data and the animal data to create, modify, or enhance at least one primary insight related to at least one physiological-based condition of the targeted individual. The computing subsystem is further operable to make one or more modifications or enhancements to the at least one primary insight as additional animal data, additional contextual data, or a combination thereof, is gathered by the computing subsystem. The computing subsystem is also configured to access at least one reference insight. The at least one reference insight and the at least one primary insight are used to create, modify, or enhance at least one predictive indicator. Lastly, the at least one predictive indicator is used by one or more computing devices to at least one of: (1) create, modify, enhance, or evaluate one or more odds; (2) create, modify, enhance, or evaluate one or more bets; (3) as a market upon which one or more bets are placed or accepted; (4) formulate one or more strategies; (5) create, modify, enhance, acquire, offer, or distribute one or more products; or (6) mitigate, prevent, or take one or more risks.

In another aspect, a system for generating dynamic real-time predictions using heart rate variability is described. The system includes one or more source sensors that gather animal data from a targeted individual in the context of one or more events (e.g., animal data gathered prior to, during, or after one or more events), the animal data inclusive of heart rate variability data or animal data from which heart rate variability can be derived. The system is configured to also gather contextual data and event outcome data from one or more sources (e.g., via one or more sensors or sensing systems, one or more other computing devices, one or more lines of code that enable the input of data, and the like). The system is trained using one or more artificial intelligence techniques to understand each of the one or more biological functions that occur with the targeted individual's body (e.g., biological processes or responses in the body; physical occurrences or responses from the body) (either voluntarily or involuntarily) based upon the gathered animal data, the context (e.g., of the event) in which the animal data was gathered (i.e., contextual data), and the event outcome associated with the biological occurrence (i.e., event outcome data), at least a portion of which becomes part of a reference animal database. The system is further trained to associate each of the one or more biological functions with the targeted individual, the contextual data, and the event outcome, creating a personalized understanding of each targeted individual and their one or more biological functions for each event, sub-event, or a subset of events. The personalized understanding of each targeted individual and their one or more biological functions for each event, sub-event, or a subset of events enables the system to create, modify, or enhance at least one baseline for each targeted individual on a per-event or multi-event basis. Characteristically, the system is configured to weigh various aspects of the gathered data (e.g., one or more variables) using the one or more Artificial Intelligence techniques. The weight of these various aspects is distributed based upon the strength of the correlation between the reference animal data and the event outcome. In some variations, the system may utilize one or more data sets from one or more other individuals to determine the weight, at least in part. Based upon a comparison with the at least one baseline, the system uses the real-time animal data gathered from the targeted individual via one or more source sensors (or one or more derivatives of the animal data) and gathered contextual data to make one or more predictions related to an event outcome for one or more targeted events. Characteristically, the one or more predictions can occur dynamically and in real-time or near real-time based upon new animal data being gathered the system from the one or more source sensors. The system is configured to observe the real-world event outcomes and learn from the gathered animal data, contextual data, and event outcome data in order to measure the performance (e.g., evaluate the accuracy) of the one or more predictions and increase the accuracy of future predictions.

In another aspect, a system for generating dynamic real-time predictions using heart rate variability is provided. The system includes one or more sensors that gather as gathered animal data from a targeted individual associated with an event. At least a portion of the gathered animal data is heart rate variability. Characteristically, animal data is transmitted by the one or more sensors electronically. A transmission subsystem receives animal data from the sensors and provides transmitted animal data to a computing subsystem. A computing subsystem gathers the animal data and contextual data associated with the gathered animal data. Advantageously, the contextual data includes event data associated with the targeted individual. The computing subsystem is configured to take one or more actions to transform the gathered animal data and contextual data into reference data. At least a portion of the reference data is used to create, modify, or enhance one or more features, the one or more features including information derived from heart rate variability data measured over an adjustable time interval. Each of the one or more features is measured in an adjustable time period prior to an outcome associated with the event being determined. The computing subsystem is also configured to organize the reference data for the targeted individual by event such that the computing subsystem implements one or more artificial intelligence-based models designed and trained with the reference data. The reference data includes the one or more features and event data, on an initial subset of one or more events associated with the targeted individual from which a predictive indicator for each event outcome is generated and compared against actual outcomes. The one or more Artificial Intelligence-based models can be further tested on a holdout data set derived from at least a portion of the event data on a rolling basis to validate the accuracy of one or more model performances. The computing subsystem is further configured to correlate one or more aspects of a targeted individual's reference data, the one or more aspects including the one or more features and the event data, to create one or more baselines for the targeted individual.

In another aspect, a system for generating dynamic real-time predictions using heart rate variability is provided. The system includes one or more source sensors that gather animal data from a targeted individual in real-time or near real-time prior to or during a targeted event. Characteristically, at least a portion of the gathered animal data is heart rate variability data. The animal data is transmitted by the one or more source sensors electronically. A transmission subsystem receives the animal data and provides transmitted animal data to a computing subsystem. A computing subsystem gathers the animal data and associated contextual data related to the gathered animal data. The associated contextual data includes event data from the targeted event associated with the targeted individual. The computing subsystem is configured to take one or more actions to transform the gathered animal data and the associated contextual data to transformed data which includes the event data from the targeted event, into a data format. At least a portion of the transformed data is used to create, modify, or enhance one or more features which include information derived from heart rate variability data measured over an adjustable time interval. Each of the features is measured in an adjustable time period prior to an outcome of the targeted event being determined. The computing subsystem is further configured to take one or more actions to transform the gathered animal data and contextual data into reference data. At least a portion of the reference data is used to create, modify, or enhance one or more features, the one or more features including information derived from heart rate variability data measured over an adjustable time interval. Each of the one or more features is measured in an adjustable time period prior to an outcome associated with the event being determined. The computing subsystem is further configured to access one or more baselines for the targeted individual prior to each targeted event. The computing subsystem utilizes the one or more baselines and the real-time or near real-time animal data or its one or more derivatives to perform one or more calculations to derive a difference in values. The computer subsystem to compare the difference in values to create, modify, or enhance at least one predictive indicator related to the outcome of the targeted event or another targeted event. The at least one predictive indicator is used by one or more computing devices to at least one of: (1) create, modify, enhance, or evaluate one or more odds; (2) create, modify, enhance, or evaluate one or more bets; (3) as a market upon which one or more bets are placed or accepted; (4) formulate one or more strategies; (5) create, modify, enhance, acquire, offer, or distribute one or more products; or (6) mitigate, prevent, or take one or more risks.

The foregoing summaries are illustrative only and are not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1 provides a schematic illustration of a method and system that uses heart rate variability-based animal data and contextual data to enable creation, modification, and enhancement of one or more dynamic real-time or near real-time predictions.

FIG. 2 provides a graph illustrating ECG measurements for a targeted individual with an increasing heart rate.

FIGS. 3A and 3B provide a graph illustrating a reference insight for two targeted individuals based upon heart rate variability data and contextual data.

FIG. 4 provides a schematic illustration of one or more transmission subsystems that can be used in conjunction with, or as part of, the method and system of FIG. 1.

FIGS. 5A and 5B provide example illustrations of comparison predictions made by the system with and without the use of animal data.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that can be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described herein, as specific components, parameters, and/or conditions may, of course, vary. Furthermore, the terminology used herein is used only for the purpose of describing particular embodiments of the present invention and is not intended to be limiting in any way.

While the terms “probability” and “odds” are mathematically different (e.g., “probability” can be defined as the number of occurrences of a certain event expressed as a proportion of all events that could occur, whereas “odds” can be defined as the number of occurrences of a certain event expressed as a proportion of the number of non-occurrences of that event), both describe the likeliness that an event will occur. They are used interchangeably to avoid redundancy, and reference to one term should be interpreted to mean reference to both.

With respect to the terms “bet” and “wager,” both terms mean an act of taking a risk (e.g., money, non-financial consideration) on the outcome of a future event. Risk includes both financial (e.g., monetary) and non-financial risk (e.g., health, life). A risk can be taken against another one or more parties (e.g., an insurance company deciding whether to provide insurance to an individual; a healthcare system deciding whether to administer one drug versus another drug, or one treatment plan versus another treatment plan, to an individual; a coach deciding whether or not to leave an athlete on the field based upon their injury history and their one or more animal data readings or derivatives thereof; and the like) or against oneself (e.g., an individual deciding whether to obtain insurance), on the basis of an outcome, or the likelihood of an outcome, of a future event. The act of making a “bet” or “wager” can occur within or as part of any system or subsystem where one or more risks can be taken, including any system where a risk is gamified (e.g., gambling, sports betting). Where the terms “bet” or “wager” are used herein, the presently disclosed and claimed subject matter can use either of the other two terms interchangeably.

It must also be noted that, as used in the specification and the appended claims, the singular form “a,” “an,” and “the” comprise plural referents unless the context clearly indicates otherwise. For example, reference to a component in the singular is intended to comprise a plurality of components.

The phrase “data is” is meant to include both “datum is” and “data are,” as well as all other possible meanings, and is not intended to be limiting in any way.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The term “one or more” means “at least one” and the term “at least one” means “one or more.” The terms “one or more” and “at least one” include “plurality” and “multiple” as a subset. In a refinement, “one or more” includes “two or more.”

The term “derivative” wherein referring to data means that the data is mathematically transformed to produce the derivative as an output. In a refinement, a mathematic function receives the data as input and outputs the derivative as an output.

The term “or its one or more derivatives” can be interchangeable with “and its one or more derivatives” depending on the use case and is not intended to be limiting in any way.

Throughout this application, where publications are referenced, the disclosures of these publications in their entireties are hereby incorporated by reference into this application to more fully describe the state of the art to which this invention pertains.

The term “server” refers to any computer or computing device (including, but not limited to, desktop computer, notebook computer, laptop computer, mainframe, mobile phone, smart watch, smart contact lens, head-mountable units such as smart glasses, headsets such as augmented reality headsets, virtual reality headsets, mixed reality headsets, and the like, hearables, augmented reality devices, virtual reality devices, mixed reality devices, and the like), distributed system, blade, gateway, switch, processing device, or a combination thereof adapted to perform the methods and functions set forth herein.

The term “computing device” refers generally to any device that can perform at least one function, including communicating with another computing device. In a refinement, a computing device includes a central processing unit that can execute program steps and memory for storing data and a program code.

When a computing device is described as performing an action or method step, it is understood that the one or more computing devices are operable to and/or configured to perform the action or method step typically by executing one or more lines of source code. The actions or method steps can be encoded onto non-transitory memory (e.g., hard drives, optical drive, flash drives, and the like).

The term “electronic communication” means that an electrical signal is either directly or indirectly sent from an originating electronic device to a receiving electronic device. Indirect electronic communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electronic communication can be accomplished with wired components, wirelessly-connected components, or a combination thereof.

The processes, methods, or algorithms disclosed herein can be deliverable to or implemented by a computer, controller, or other computing device, which can include any existing programmable electronic control unit or dedicated electronic control unit. Similarly, the processes, methods, or algorithms can be stored as data and instructions executable by a computer, controller, or other computing device in many forms including, but not limited to, information permanently stored on non-writable storage media such as ROM devices and information alterably stored on writeable storage media such as floppy disks, magnetic tapes, CDs. RAM devices, other magnetic and optical media, and shared or dedicated cloud computing resources. The processes, methods, or algorithms can also be implemented in an executable software object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software, and firmware components.

The terms “subject” and “individual” are synonymous, interchangeable, and refer to a human or other animal, including birds, reptiles, amphibians, and fish, as well as all mammals including, but not limited to, primates (particularly higher primates), horses, sheep, dogs, rodents, pigs, cats, rabbits, and cows. The one or more subjects or individuals can be, for example, humans participating in athletic training or competition, horses racing on a race track, humans playing a video game, humans monitoring their personal health, humans providing their animal data to a third party (e.g., insurance system, health system, monetization system), humans participating in a research or clinical study, humans participating in a fitness class, and the like. A subject or individual can also be a derivative of a human or other animal (e.g., lab-generated organism derived at least in part from a human or other animal), one or more individual components, elements, or processes of a human or other animal (e.g., cells, proteins, biological fluids, amino acid sequences, tissues, hairs, limbs) that make up the human or other animal, one or more digital representations that share at least one characteristic with a human or other animal (e.g., data set representing a human that shares at least one characteristic with a human representation in digital form—such as sex, age, biological function as examples—but is not generated from any human that exists in the physical world; a simulated individual or digital individual that is based on, at least in part, a real-world human or other animal, such as a digital representation of an individual or avatar in a virtual environment or simulation such as a video game or metaverse), or one or more artificial creations that share one or more characteristics with a human or other animal (e.g., lab-grown human brain cells that produce an electrical signal similar to that of human brain cells). In a refinement, the subject or individual can be one or more programmable computing devices such as a machine (e.g., robot, autonomous vehicle, mechanical arm) or network of machines that share at least one biological function with a human or other animal and from which one or more types of biological data can be derived, which can be, at least in part, artificial in nature (e.g., data from Artificial Intelligence-derived activity that mimics biological brain activity; biomechanical movement data derived a programmable machine that mimics, at least in part, biomechanical movement of an animal).

The term “animal data” refers to any data obtainable from, or generated directly or indirectly by, a subject that can be transformed into a form that can be transmitted to a server or other computing device. Typically, the animal data is electronically transmitted via a wired or wireless connection. Animal data includes, but is not limited to, any subject-derived data, including any signals or readings (e.g., metrics), that can be obtained from one or more sensors or sensing equipment/systems, and in particular, biological sensors (i.e., biosensors) that produce biological data, as well as its one or more derivatives. Animal data also includes any biological phenomena capable of being captured from a subject and converted to electrical signals that can be captured by one or more sensors, descriptive data related to a subject (e.g., name, age, height, eye-color, gender, anatomical information), auditory data related to a subject, visually-captured data related to a subject (e.g., image, likeness, observable information related to the subject), neurologically-generated data (e.g., brain signals from neurons), evaluative data related to a subject (e.g., skills of a subject), data that can be manually entered or gathered related to a subject (e.g., medical history, social habits, feelings of a subject, mental health data, financial information, subjective data), and the like (e.g., attributes/characteristics of the individual). The term “animal data” can be meant to include one or more types of animal data. In a refinement, the term “animal data” is inclusive of any derivative of animal data, including one or more reference insights (e.g., including historical reference insights), primary insights (e.g., including historical primary insights), predictive indicators (e.g., including reference predictive indicators), artificial data (e.g., simulated animal data in a virtual environment, video game, or other simulation derived from the digital representation of the subject), or a combination thereof. In another refinement, animal data includes one or more inputs (e.g., signals, readings, other data) from one or more non-animal data sources. In another refinement, animal data includes at least a portion of non-animal data that provides contextual information related to the animal data. In another refinement, animal data includes any metadata gathered or associated with the animal data. In another refinement, animal data includes at least a portion of simulated data. In yet another refinement, animal data is inclusive of simulated data.

The term “reference animal data” refers to any animal data used as a reference or baseline (e.g., a base for measurement) to classify, categorize, or evaluate (e.g., compare, analyze) other animal data, as well as to derive information from other data. It can include any available, accessible, or gathered data, including any type of animal data and/or non-animal data, either directly or indirectly related to (or derived from) one or more targeted subjects or events associated with the one or more targeted subjects that enables one or more forecasts, predictions, probabilities, assessments, possibilities, projections, determinations, or recommendations related to one or more outcomes for one or more current or future events or sub-events to be calculated, computed, derived, extracted, extrapolated, quantified, simulated, created, modified, assigned, enhanced, estimated, evaluated, inferred, established, determined, converted, deduced, observed, communicated, or actioned upon. Reference animal data can be gathered from any number of subjects (e.g., one, tens, hundreds, thousands, millions, billions, and the like) and data sources (e.g., it can be gathered from sensors or computing devices, manually inputted, artificially created, derived from one or more actions, and the like). It can be structured (e.g., created, curated) in a way to facilitate one or more evaluations (e.g., comparisons) of (or between) data sets and/or derivatives of data sets. Reference animal data can also include any (i) previously-collected animal data (e.g., historical animal data), including derivatives from animal data (e.g., one or more reference insights, historical reference insights, primary insights, historical primary insights, predictive indicators, or reference predictive indicators) and previously-collected animal data derived from one or more sensors, as well as (ii) created derivatives from animal data. In some variations, it can also include associated contextual data, which can include other animal data, non-animal data (e.g., including non-animal data directly or indirectly related to (or associated with) the previously-collected animal data), or a combination thereof. In a refinement, reference animal data includes at least a portion of the previously collected animal data derived from one or more sensors. In another refinement, reference animal data includes at least a portion of non-animal data (e.g., including non-animal contextual data to provide additional context to the animal data). In another refinement, reference animal data is stored, categorized, and accessed by the system with associated reference contextual data. In another refinement, reference animal data has associated reference contextual data which comprises, at least in part, the reference animal data. In another refinement, reference animal data includes at least a portion of simulated animal data (e.g., the system may generate artificial animal data as reference animal data; the system may run one or more simulations, the output of which can be reference animal data; one or more animal data sets may include simulated data; and the like). In another refinement, reference animal data includes metadata gathered or associated with animal data (e.g., contextual data, reference contextual data). In another refinement, reference animal data for a targeted individual includes any animal data that is derived directly from the targeted individual, indirectly from the targeted individual, or a combination thereof. In another refinement, reference animal data for a targeted individual includes data that is not derived directly or indirectly from the targeted individual but shares at least one attribute (e.g., characteristic) with the one or more targeted individuals or biological responses. In another refinement, reference animal data can include identifiable, de-identified (e.g., pseudonymized), semi-anonymous, or anonymous data tagged with metadata (e.g., that has associated metadata) related to one or more biological responses. In another refinement, reference animal data includes data derived from the one or more biological responses derived from anonymized, semi-anonymized, or de-identified (e.g., pseudonymized) sources. In a variation, reference animal data can be categorized, or grouped together, to form one or more units of such data. In another refinement, reference animal data can be dynamically created, modified, or enhanced with one or more additions, changes, or removal of non-functioning data (e.g., data that the system will remove or stop using). In another refinement, at least a portion of the reference animal data can be weighted based upon one or more characteristics of (or related to) the one or more sensors (e.g., reference animal data from sensors that produce average quality data may have a lower weighted score than reference animal data from sensors that produce high quality data), the one or more individuals or groups of individuals, the contextual data associated with the animal data (e.g., other animal data, non-animal data), or a combination thereof. In another refinement, the system can be operable to conduct one or more data audits on reference animal data. For example, the system may recall reference animal data originating from one or more sensors based upon one or more sensor characteristics (e.g., a faulty data gathering functionality within the one or more sensors could cause the system to recall and remove the data from the reference animal data database to enable more accurate predictions). In another refinement, the reference animal data includes previously collected animal data that are typically analyzed and characterized.

In a refinement, the term “reference animal data” is synonymous and used interchangeably with the term “reference data.” and a reference to any one of the terms should not be interpreted as limiting but rather as encompassing all possible meanings of all the terms. In another variation, the term “reference data” includes reference animal data, reference contextual data, or a combination thereof.

The term “artificial data” refers to artificially-created data that is derived from, based on, or generated using, at least in part, animal data or one or more derivatives thereof. It can be created by running one or more simulations utilizing one or more Artificial Intelligence (“AI”) techniques or statistical models, and can include one or more inputs (e.g., signals, readings, other data) from one or more non-animal data sources. In a refinement, artificial data includes any artificially-created data that shares at least one biological function with a human or another animal (e.g., artificially-created vision data, artificially-created movement data). The term “artificial data” is inclusive of “synthetic data,” which can be any production data applicable to a given situation that is not obtained by direct measurement. Synthetic data can be created by statistically modeling original data and then using the one or more models to generate new data values that reproduce at least one of the original data's statistical properties. In another refinement, the term “artificial data” is inclusive of any derivative of artificial data. In another refinement, artificial data is generated utilizing at least a portion of reference animal data. For the purposes of the presently disclosed and claimed subject matter, the terms “simulated data” and “synthetic data” are synonymous and used interchangeably with “artificial data” (and vice versa), and a reference to any one of the terms should not be interpreted as limiting but rather as encompassing all possible meanings of all the terms. In another refinement, the term “artificial data” is inclusive of the term “artificial animal data.”

The term “predictive indicator” refers to a metric or other indicator (e.g., one or more colors, codes, numbers, values, graphs, charts, plots, readings, numerical representations, descriptions, text, physical responses, auditory responses, visual responses, kinesthetic responses) derived from at least a portion of animal data from which one or more forecasts, predictions, probabilities, assessments, possibilities, projections, or recommendations related to one or more outcomes for one or more future events or sub-events that includes one or more targeted individuals, or one or more groups of targeted individuals, can be calculated, computed, derived, extracted, extrapolated, quantified, simulated, created, modified, assigned, enhanced, estimated, evaluated, inferred, established, determined, converted, deduced, observed, communicated, or actioned upon. In a refinement, a predictive indicator is derived, at least in part, either directly or indirectly from the comparison between the at least one primary insight and the at least one reference insight. In another refinement, a predictive indicator includes one or more inputs (e.g., signals, readings, other data) from one or more non-animal data sources as one or more inputs in the one or more calculations, computations, combinations, measurements, derivations, extractions, extrapolations, simulations, creations, modifications, assignments, enhancements, estimations, evaluations, inferences, establishments, determinations, conversions, deductions, observations, or communications of its one or more forecasts, predictions, probabilities, possibilities, assessments, projections, or recommendations. In another refinement, a predictive indicator includes at least a portion of simulated data as one or more inputs in the one or more calculations, computations, combinations, measurements, derivations, extractions, extrapolations, simulations, creations, modifications, assignments, enhancements, estimations, evaluations, inferences, establishments, determinations, conversions, deductions, observations, or communications of its one or more forecasts, predictions, probabilities, possibilities, assessments, projections, or recommendations. In another refinement, a predictive indicator is derived from two or more types of animal data. In another refinement, a predictive indicator is comprised of a plurality of predictive indicators. In yet another refinement, a created, modified, or enhanced predictive indicator is used as training data for one or more Artificial Intelligence-based techniques to create, modify, or enhance of one or more subsequent predictive indicators.

For the purposes of this invention, any reference to the collection or gathering of animal data from one or more source sensors from a subject includes gathering the animal data from one or more computing devices associated with the one or more source sensors (e.g., a cloud server or other computing device associated with the one or more source sensors where the data is stored or accessible). Additionally, the terms “gathering” and “collecting” can be used interchangeably, and reference to any one of the terms should not be interpreted as limiting but rather as encompassing all possible meanings of both terms. In a refinement, the terms “gathering” and “collecting” can be used interchangeably with the term “receiving” (and vice versa), and reference to any one of the terms should not be interpreted as limiting but rather as encompassing all possible meanings of all the terms.

The term “modify” can be inclusive of “revise.” “amend,” “adjust,” “update,” “change,” and “refine” (and vice versa). Additionally, the term “create” can be inclusive of “derive” and vice versa. Similarly, “create” can be inclusive of “generate” and vice versa. In a refinement, “create” can also include an action that is calculated, computed, derived, extracted, extrapolated, simulated, modified, enhanced, estimated, evaluated, inferred, established, determined, converted, or deduced. The term “enhance” refers to an improvement of quality or value in data and in particular the animal data or one or more derivatives thereof (e.g., unique asset, predictive indicator, insight).

A modification or enhancement of data can occur (1) as new data (e.g., animal data, non-animal data) is gathered by the system; (2) based upon one or more evaluations of existing data (e.g., one or more new patterns, trends, features, measurements, outliers, abnormalities, anomalies, readings, signals, data sets, characteristics/attributes, and the like that are identified in existing data sets by the system); (3) as existing data is removed or replaced in the system; (4) as the system learns one or more new methods of transforming existing data into new data sets or deriving new data sets from existing data (e.g., the system learns to derive respiration rate data from raw sensor data that is traditionally used to extrapolate ECG data); (5) as new data is generated artificially; and/or (6) as a result of one or more simulations; and the like. For example, new data entering the system may enhance the accuracy of the system's predictive indicator. In another example, a data set or animal data derivative can be modified if data is removed from, or replaced in, the system (e.g., the system's removal of data from the reference animal data database may enable a more accurate identification of a targeted individual). In some variations, modification may result in a decrease in quality or value of the animal data or its one or more derivatives (e.g., a decrease in prediction accuracy).

The term “or a combination thereof” can mean any subset of possibilities or all possibilities. In a refinement, “or a combination thereof” includes both “or combinations thereof” and “and combinations thereof” and vice versa.

The term “neural network” refers to a Machine Learning model that can be trained with training input to approximate unknown functions. In a refinement, neural networks include a model of interconnected digital neurons that communicate and learn to approximate complex functions and generate outputs based on a plurality of inputs provided to the model.

The terms “use”, “uses”, or “used” when referring to actions taken by a computing system mean that the item being “used” is received as an input for a calculation performed by the computing system to provide an indicated output.

In a refinement, one or more comparisons or a step of comparing occur when the system utilizes one or more programs, which may incorporate one or more techniques (e.g., Artificial Intelligence techniques which can include, but are not limited to, Machine Learning techniques, Deep Learning techniques, Statistical Learning techniques, or other statistical techniques), to measure, observe, calculate, derive, extract, extrapolate, simulate, create, combine, modify, enhance, estimate, evaluate, infer, establish, determine, convert, or deduce one or more similarities, dissimilarities, or a combination thereof, between two or more animal data sets (e.g., which can include one or more derivatives of animal data and its associated metadata), at least one of which is derived from reference animal data and at least one of which is derived—at least in part—from one or more source sensors. In a further refinement, at least one of the two or more animal data sets incorporates at least one primary insight and at least one of the two or more animal data sets incorporates at least one reference insight to enable the creation, modification, or enhancement of at least one predictive indicator. In one scenario, a comparison occurs when the system utilizes a sophisticated ensemble clustering algorithm that uses a combination of clustering algorithms that can include Density-Based Spatial Clustering Of Applications With Noise (DBSCAN), BIRCH, Gaussian Mixture Model (GMM), Hierarchical Clustering Algorithm (HCA) and Spectral-based clustering while using metrics of similarity grouping that can include inertia and silhouette scoring, as well as information criteria scores to identify the group or cluster. The output of the above methodology map gives data to a cluster or group. Within the identified group, one or more additional Machine Learning algorithms can be used that measure the nearness of data to similar sub-groups to identify, at least in part, the potential target the given data belongs to. In another refinement, “compare” can mean “evaluate” and/or “analyze,” and vice versa. For example, a step of comparing a primary insight and a reference insight to create one or more predictive indicators can involve forming insights for a target individual from R-R intervals. Reference insights can be created in which predetermined ranges of values are associated with predefined primary insights. Therefore, in this context, “compare” means to select the primary insight corresponding to the range measured from an individual.

### Abbreviations:

“AI” means artificial intelligence.

“CNN” means convolutional neural networks.

“DBSCAN” means Density-Based Spatial Clustering Of Applications With Noise.

“GMM” means Gaussian Mixture Model.

“HCA” means Hierarchical Clustering Algorithm.

“HF” means high-frequency.

“HRV” means heart rate variability.

“IBI” means Interbeat Intervals.

“LF” means low-frequency.

“PCA” means principal component analysis.

“PPG” means photoplethysmogram.

“PRED” means prediction.

“RMSSD” means root mean square of successive differences between normal heartbeats.

“R-R” or “R-R interval” means the time elapsed between two successive R waves of the QRS signal on the electrocardiogram.

“ULF” means ultra-low-frequency.

“VLF” means very-low-frequency.

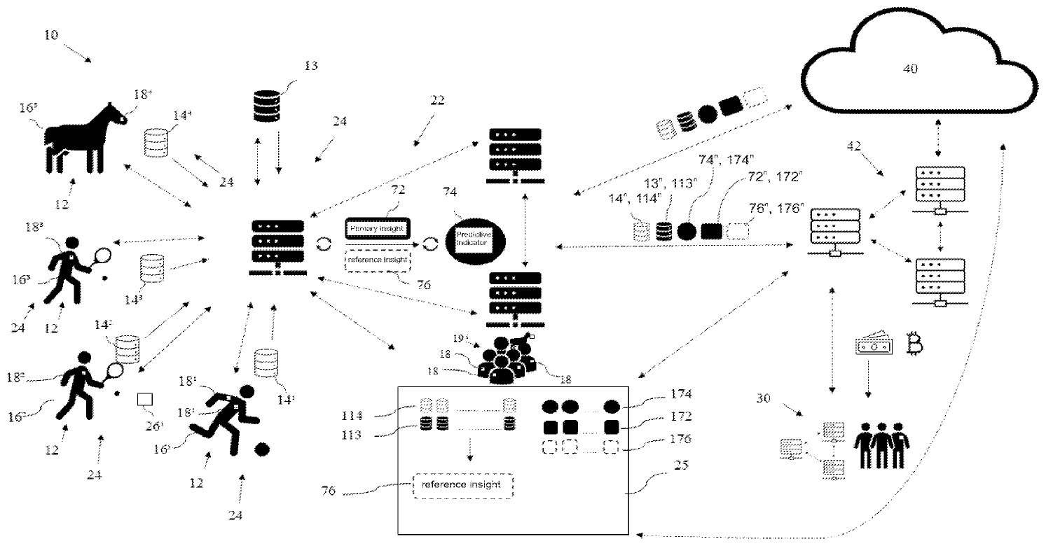

With reference to FIG. 1, a schematic of a method and system for generating dynamic real-time predictions using heart rate variability is provided. Prediction system 10 includes a source 12 of animal data 14 that can be transmitted electronically. In this context, transmitted electronically includes being provided in an electronic form. In some variations, source 12 of animal data 14 refers to data related to targeted individual 16ⁱ. Targeted individual 16ⁱ is the subject from which corresponding animal data 14 is collected. Label i is merely an integer label from 1 to iₘₐₓ associated with each targeted individual where iₘₐₓ is the total number of individuals, which can be 1 to several thousand to several million or more. In this context, animal data can refer to any data related to a subject. In some embodiments, animal data refers to data related to a subject's body derived, at least in part, from one or more sensors and, in particular, biological sensors (also referred to as biosensors). Therefore, in these embodiments the one or more sources 12 of animal data 14 includes one or more sensors. In many useful applications, targeted individual 16ⁱ is a human (e.g., an athlete, a soldier, a healthcare patient, a research subject, a participant in a fitness class, a video gamer) and the animal data 14 is human data.

Animal data can be derived from (e.g., collected from) a targeted individual or multiple targeted individuals (e.g., including a targeted group of multiple targeted individuals, multiple targeted groups of multiple targeted individuals). In the case of sensors, the animal data can be obtained from a single sensor 18 gathering information from each targeted individual 16ⁱ, or from multiple sensors 18 gathering information from each targeted individual 16ⁱ. Each sensor 18 gathering animal data from source 12 of animal data 14 from targeted individual 16ⁱ can be classified as a source sensor. In some cases, a single sensor can capture data from multiple targeted individuals, a targeted group of multiple targeted individuals, or multiple targeted groups of multiple targeted individuals (e.g., an optical-based camera sensor that can locate and measure distance run or respiratory data for a targeted group of targeted individuals). Each sensor can provide a single type of animal data or multiple types of animal data. In a variation, sensor 18 can include multiple sensing elements to measure one or more parameters within a single sensor (e.g., heart rate and accelerometer data). One or more sensors 18 can collect data from a targeted individual engaged in a variety of activities including strenuous activities that can change one or more biological signals or readings in a targeted individual such as blood pressure, heart rate, or biological fluid levels. Activities may also include sedentary activities such as sleeping or sitting where changes in biological signals or readings may have less variance. One or more sensors 18 can also collect data before or after one or more other activities (e.g., after a run, after waking up, after ingesting one or more substances or medications, and any other activity suitable for data collection from one or more sensors). In a refinement, one or more sensors 18 can be classified as a computing device with one or more computing capabilities. In a variation, prediction system 10 can also gather (e.g., receive, collect) animal data not obtained from sensors (e.g., animal data that is inputted or gathered via a computing device; animal data sets that include artificial data values not generated directly from a sensor; animal data received from another computing device). This can occur via computing subsystem 22 or via one or more other computing devices in communication with computing subsystem 22 that gather animal data (e.g., computing device 26). In a refinement, one or more sensors 18 are operable to collect at least a portion of non-animal data. In another refinement, at least one sensor of the one or more source sensors 18 captures two or more types of animal data. In another refinement, at least one sensor of the one or more source sensors 18 is comprised of two or more sensors. In another refinement, the one or more sensors 18 can collect data over a continuous period of time or at regular or irregular intervals. In many variations, one or more sensors 18 are operable for real-time or near real-time communication. In another refinement, at least one of the one or more sensors 18 are operable to provide streaming animal data. In a variation, one or more sensor functionalities, parameters, or properties are operable to be configured either directly or indirectly (e.g., via another one or more other computing devices) by the system.

One or more sensors 18 can include one or more biological sensors (also referred to as biosensors). Biosensors collect biosignals, which in the context of the present embodiment are any signals or properties in, or derived from, animals that can be continually or intermittently measured, monitored, observed, calculated, computed, or interpreted, including both electrical and non-electrical signals, measurements, and artificially-generated information. A biosensor can gather biological data (e.g., including readings and signals, both in raw or manipulated/processed form) such as physiological data, biometric data, chemical data, biomechanical data, genetic data, genomic data, glycomic data, location data or other biological data from one or more targeted individuals. For example, some biosensors may measure, or provide information that can be converted into or derived from, biological data such as eye tracking & recognition data (e.g., pupillary response, movement, pupil diameter, iris recognition, retina scan, eye vein recognition, EOG-related data), blood flow data and/or blood volume data (e.g., photoplethysmogram (PPG) data, pulse transit time, pulse arrival time), biological fluid data (e.g., analysis derived from blood, urine, saliva, sweat, cerebrospinal fluid), body composition data (e.g., bioelectrical impedance analysis, weight-based data including weight, body mass index, body fat data, bone mass data, protein data, basal metabolic rate, fat-free body weight, subcutaneous fat data, visceral fat data, body water data, metabolic age, skeletal muscle data, muscle mass data), pulse data, oxygenation data (e.g., SpO2), core body temperature data, galvanic skin response data, skin temperature data, perspiration data (e.g., rate, composition), blood pressure data (e.g., systolic, diastolic, MAP), glucose data (e.g., fluid balance I/O, glycogen usage), hydration data (e.g., fluid balance I/O), heart-based data (e.g., heart rate, average HR, HR range, heart rate variability, HRV time domain, HRV frequency domain, autonomic tone, ECG-related data including PR, QRS, QT, R-R intervals, echocardiogram data, thoracic electrical bioimpedance data, transthoracic electrical bioimpedance data), neurological data and other neurological-related data (e.g., EEG-related data), genetic-related data (e.g., performance enhancing polymorphisms (PEPs) such as ACTN3, ACE, ADRB2, AMPD1, BDKRB2, APOE, and others), genomic-related data, skeletal data, muscle data (e.g., EMG-related data including surface EMG, amplitude, adenosine triphosphate (ATP) data, muscle fiber types, muscle contraction velocity, muscle elasticity, soft-tissue strength), respiratory data (e.g., respiratory rate, respiratory pattern, inspiration/expiration ratio, tidal volume, spirometry data), and the like. Some biosensors may detect biological data such as biomechanical data which may include, for example, angular velocity, joint paths, kinetic or kinematic loads, gait description, step count, reaction time, or position or accelerations in various directions from which a subject's movements can be characterized. Some biosensors may gather biological data such as location and positional data (e.g., GPS, ultra-wideband RFID-based data; posture data), facial recognition data, posterior profiling data, audio data, kinesthetic data (e.g., physical pressure captured from a sensor located at the bottom of a shoe), other biometric authentication data (e.g., fingerprint data, hand geometry data, voice recognition data, keystroke dynamics data—including usage patterns on computing devices such as mobile phones, signature recognition data, ear acoustic authentication data, eye vein recognition data, finger vein recognition data, footprint and foot dynamics data, body odor recognition data, palm print recognition data, palm vein recognition data, skin reflection data, thermography recognition data, speaker recognition data, gait recognition data, lip motion data), or auditory data (e.g., speech/voice data, sounds made by the subject, emotion captured derived from verbal tone or words used) related to the one or more targeted individuals. Some biological sensors can be image or video-based and collect, provide and/or analyze video or other visual data (e.g., still or moving images, including video, MRIs, computed tomography scans, ultrasounds, echocardiograms, X-rays) upon which biological data can be detected, measured, monitored, observed, extrapolated, calculated, or computed (e.g., biomechanical movements or location-based information derived from video data, a fracture detected based on an X-Ray, or stress or a disease of a subject observed based on video or image-based visual analysis of a subject; observable animal data such as facial movements, bodily movements or a wince which can indicate pain or fatigue). Some biosensors may derive information from biological fluids such as blood (e.g., venous, capillary), saliva, urine, sweat, and the like including (but not limited to) triglyceride levels, red blood cell count, white blood cell count, adrenocorticotropic hormone levels, hematocrit levels, platelet count, ABO/Rh blood typing, blood urea nitrogen levels, calcium levels, carbon dioxide levels, chloride levels, creatinine levels, glucose levels, hemoglobin A1c levels, lactate levels, sodium levels, potassium levels, bilirubin levels, alkaline phosphatase (ALP) levels, alanine transaminase (ALT) levels, and aspartate aminotransferase (AST) levels, albumin levels, total protein levels, prostate-specific antigen (PSA) levels, microalbuminuria levels, immunoglobulin A levels, folate levels, cortisol levels, amylase levels, lipase levels, gastrin levels, bicarbonate levels, iron levels, magnesium levels, uric acid levels, folic acid levels, vitamin B-12 levels, and the like. In a variation, some biosensors may collect biochemical data including acetylcholine data, dopamine data, norepinephrine data, serotonin data, GABA data, glutamate data, hormonal data, and the like. In addition to biological data related to one or more targeted individuals, some biosensors may measure non-biological data (e.g., ambient temperature data, humidity data, elevation data, barometric pressure data, and the like). In a refinement, one or more sensors provide biological data that include one or more calculations, computations, combinations, predictions, probabilities, possibilities, estimations, evaluations, inferences, determinations, deductions, observations, or forecasts that are derived, at least in part, from animal data. In another refinement, the one or more biosensors are capable of providing at least a portion of artificial data. In another refinement, the one or more biosensors are capable of providing two or more types of data, at least one of which is biological data (e.g., heart rate data and VO2 data, muscle activity data and accelerometer data, VO2 data and elevation data, or the like). In a refinement, the one or more sensors is a biosensor that gathers physiological, biometric, chemical, biomechanical, location, environmental, genetic, genomic, glycomic, or other biological data from one or more targeted individuals. In another refinement, one or more biosensors collect image data and/or video data (e.g., one or more images of the subject, one or more videos of the subject, or a combination thereof) via one or more image sensors, video sensors, or a combination thereof.

In another refinement, at least one sensor 18 and/or its one or more appendices thereof can be affixed to, are in contact with, or send one or more electronic communications in relation to or derived from, one or more targeted subjects including the one or more targeted subjects' body, skin, eyeball, vital organ, muscle, hair, veins, biological fluid, blood vessels, tissue, or skeletal system, embedded in one or more targeted subjects, lodged or implanted in one or more targeted subjects, ingested by one or more targeted subjects, or integrated to include at least a subset of one or more targeted subjects. For example, a saliva sensor affixed to a tooth, a set of teeth, or an apparatus that is in contact with one or more teeth, a sensor that extracts DNA information derived from a targeted subject's biological fluid or hair, sensor that is wearable (e.g., on a human or other animal body), a sensor in a computing device (e.g., phone) that is tracking a targeted individual's location information or collecting other biometric information (e.g., facial recognition, voice, fingerprint), one or more sensors integrated within a head-mountable unit such as smart glasses or a virtual/augmented/mixed reality headset that track eye movements and provide eye tracking data and recognition data, one or more sensors that are integrated into one or more computing devices that analyze biological fluid data, a sensor affixed to or implanted in the targeted subject's brain that may detect brain signals from neurons, a sensor that is ingested by a targeted subject to track one or more biological functions, a sensor attached to, or integrated with, a machine (e.g., robot) that shares at least one characteristic with an animal (e.g., a robotic arm with an ability to perform one or more tasks similar to that of a human; a robot with an ability to process information similar to that of a human), and the like. Advantageously, the machine itself can include one or more sensors, and can be classified as both a sensor and a subject. In another refinement, the one or more sensors 18 are integrated into or as part of, affixed to, or embedded within, a textile, fabric, cloth, material, fixture, object, or apparatus that contacts or is in communication with a targeted individual either directly or via one or more intermediaries or interstitial items. Examples include, but are not limited to, a sensor attached to the skin via an adhesive, a sensor integrated into a watch or head-mountable or wearable unit (e.g., augmented reality or virtual reality headset, smart glasses, hat, headband), a sensor integrated or embedded into clothing (e.g., shirt, jersey, shorts, wristband, socks, compression gear), a sensor integrated into a steering wheel, a sensor integrated into a computing device controller (e.g., video game or virtual environment controller, augmented reality headset controller, remote control for media), a sensor integrated into a ball that is in contact with an extremity of a targeted subject's body such as their hands (e.g. basketball) or feet (e.g., soccer), a sensor integrated into a ball that is in contact with an intermediary being held by the targeted subject (e.g., bat), a sensor integrated into a hockey stick or a hockey puck that is in intermittent contact with an intermediary being held by the targeted subject (e.g., hockey stick), a sensor integrated or embedded into the one or more handles or grips of fitness equipment (e.g., treadmill, bicycle, row machine, bench press, dumbbells), a sensor that is integrated within a robot (e.g., robotic arm) that is being controlled by the targeted individual, a sensor integrated or embedded into a shoe that may contact the targeted individual through the intermediary sock and adhesive tape wrapped around the targeted individual's ankle, and the like. In another refinement, one or more sensors can be interwoven into, embedded into, integrated with, or affixed to, a flooring or ground (e.g., artificial turf, grass, basketball floor, soccer field, a manufacturing/assembly-line floor, yoga mat, modular flooring), a seat/chair, helmet, a bed, an object that is in contact with the targeted subject either directly or via one or more intermediaries (e.g., a subject that is in contact with a sensor in a seat via a clothing intermediary), and the like. In another refinement, one or more sensors can be integrated with or affixed to one or more aerial apparatus such as an unmanned aerial vehicle (e.g., drone, high-altitude long-endurance aircraft, a high-altitude pseudo satellite (HAPS), an atmospheric satellite, a high-altitude balloon, a multirotor drone, an airship, a fixed-wing aircraft, or other altitude systems) or other aerial computing device that utilize one or more sensors (e.g., optical, infrared) to collect animal data (e.g., skin temperature, body temperature, heart rate, heart rate variability, respiratory rate, facial recognition, gait recognition, location data, image data, one or more subject characteristics or attributes, and the like) from one or more targeted subjects or groups of targeted subjects. In another refinement, the sensor and/or its one or more appendices can be in contact with one or more particles or objects derived from the targeted subject's body (e.g., tissue from an organ, hair from the subject) from which the one or more sensors derive, or provide information that can be converted into, biological data. In yet another refinement, one or more sensors can be optically-based (e.g., camera-based) and provide an output from which biological data can be detected, measured, monitored, observed, extracted, extrapolated, inferred, deducted, estimated, determined, combined, calculated, or computed. In yet another refinement, one or more sensors can be light-based and use infrared technology (e.g., temperature sensor or heat sensor) to gather or calculate biological data (e.g., skin or body temperature) from an individual or the relative heat of different parts of an individual. In yet another refinement, the one or more sensors gather animal data related to one or more attributes/characteristics or states of being of an individual (e.g., an optical sensor that gathers animal data such as skin color, facial hair, eye color, conditions of the skin, and the like; an optical sensor that detects pain, fatigue, injury, a medical event/episode/condition, and the like).

Still referring to FIG. 1, each individual 16ⁱ has at least one sensor 18 that gathers animal data 14 from the targeted individual 16ⁱ. In one variation, animal data 14 includes heart rate variability data. In another variation, Animal data 14 includes animal data from which heart rate variability can be derived. Heart rate variability (HRV) is the variation between each heartbeat and can be computed based upon (e.g., from) the Interbeat Intervals (IBI). HRV can be measured using a variety of methods and techniques including time-domain (e.g. AVNN, SDNN, pNN50, RMSSD), frequency domain (e.g., ultra-low-frequency (ULF), very-low-frequency (VLF), low-frequency (LF), and high-frequency (HF) bands), non-linear analysis, and other methods. The inventors prefer using the root mean square of successive differences between normal heartbeats (RMSSD), which provides a measure of parasympathetic nervous system activity, to generate dynamic real-time predictions using HRV, although other techniques, methodologies, or formulas can be utilized to generate similar outputs. A normal heartbeat can be the heartbeat of a subject at rest or prior to an event being analyzed. A normal heartbeat can be the heartbeat of a subject during warmup for an event or the heartbeat during a prior event.

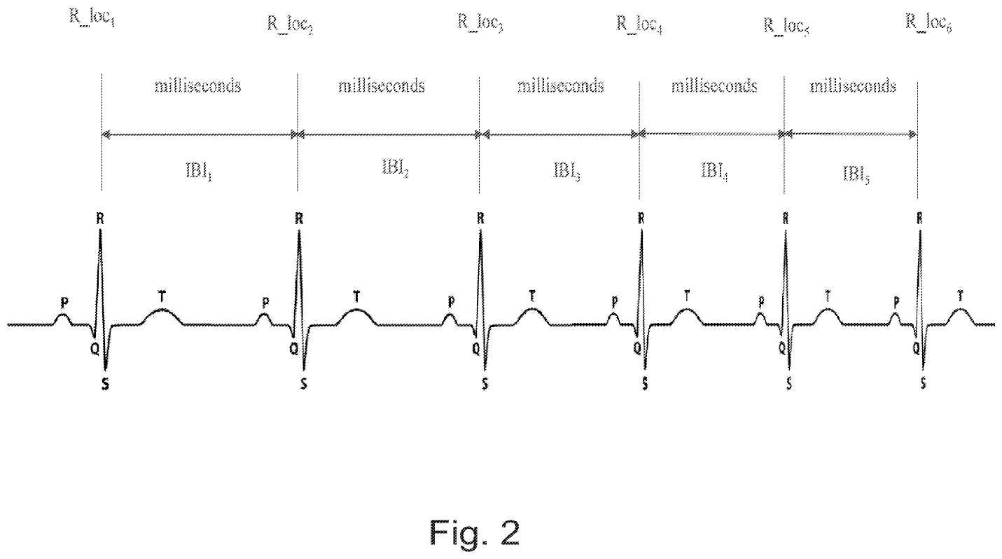

FIG. 2 illustrates an exemplary output of the ECG measurements from which heart rate variability can be derived. Note that the invention is not limited to ECG-based HRV measurements and can also use HRV derived from other methods (e.g., PPG-based). Various points in the repeating pattern are labelled P, Q. R. S, and T. The R point is indicated by a localized peak. The time of the R peaks are labelled R_locᵢ for 1≤i≤nmax where n is a maximum value for the integer label i. The difference between successive R_loc times are labeled Interbeat Interval i (IBIᵢ) for 1≤i<nmax. (For the output illustrated in FIG. 2, n=6.) The Interbeat Intervals—also known as R-R Intervals—are the time period between successive heartbeats and are typically measured in milliseconds. Interbeat Intervals can change based on a variety of factors (e.g., age, activity, time of day, environment, environmental conditions, mental stress, and other forms of contextual data can impact change. The time between heartbeats can also be dependent on the biological differences inherent in each individual). For example, note that the time between R peaks is shorter near the end of the time period illustrated in FIG. 2 than near the beginning of the interval. This indicates that the individual's heart rate is increasing.

In one embodiment for a method for generating dynamic real-time predictions using heart rate variability, a computing subsystem calculates one or more personalized spike counts for each targeted individual (“SDL_PERSONALIZED_SPIKE_COUNT”) to create reference animal data (e.g., historical data used as baseline data) for each targeted individual that can be used to derive one or more predictive indicators. In this context, real-time is inclusive of “near real-time,” which means any one of the steps or outputs are not purposely delayed except for necessary processing by the sensor and/or any computing device associated with embodiments of the invention. The computing subsystem executes the following series of steps:

Step 1: Gather R-R Intervals data derived from one or more sensors for a targeted individual (e.g. an athlete) prior to the start of an event for n period of time. An event can be, for example, a sports competition (e.g., any individual sports or team sports competition including a tennis match, squash match, badminton match, table tennis match, darts match, snooker match, soccer game, basketball game, football game, baseball game, hockey game, horse race, auto race, sailing race, golf event, rugby match, boxing/MMA match, eSports competition, cricket event, and the like) or an event within a sports competition (e.g., a tennis set or a tennis game within a tennis match; a tennis point within a tennis game; one or more tennis shots within a tennis point). Characteristically, an event can be comprised of one or more sub-events that feature a definable (e.g., definitive), quantifiable, observable, and/or measurable outcome (e.g., tennis shots or points can all be sub-events within a tennis match, set, or game with the outcome being whether the individual made or missed the shot or subset of shots, won or lost the point/game/set/match, or the like; in some variations, sub-events can be events and vice versa depending on the context). Time n is a tunable parameter and can be milliseconds, seconds, minutes, hours, or longer. In a refinement, an event in one instance can be classified a sub-event in another instance (and vice versa) depending on the context or the event.

Note that for the purposes of teaching the invention, the inventors illustrate the invention in the context of an individual athlete sports competition. However, the invention is not limited to any particular individual athlete or team sports competition and can be implemented across all sports—including both individual athlete sports and team sports, across one or more events within each sport, and across sub-events within each event. It should also be appreciated that the invention is not limited to sports applications; rather, the invention can also be applied to one or more non-sports events and sub-events where there is a quantifiable, definable, observable, and/or measurable outcome (e.g., health-related applications).

Step 2: Calculate RMSSD value from the R-R Intervals data obtained above in Step 1. FIG. 2 shows the R-R intervals labeled IBI₁=(R-R)₁, IBI₂=(R-R)₂, IBI₃=(R-R)₃ . . . (R-R)ᵢ represented the interval between two neighboring QRS peaks. The RMSSD is looking for the successive difference between the intervals meaning: IBI₁-IBI₂→(R-R)₁-(R-R)₂; IBI₂-IBI₃→(R-R)₂-(R-R)₃; and the like. In one variation, RMSSD can be calculated using the following formula:

$$RMSSD = \sqrt{\frac{1}{N - 1} ( {\sum }_{i - 1}^{N - 1} {( {( R - R )}_{i + 1} - {( R - R )}_{i} )}^{2} )}$$

where N=number of R-R interval terms. The BASELINE_RMSSD is the RMSSD calculated for the n period of time (which is a tunable parameter) prior to start of the event/match/game as described in Step 1 above.

Step 3: Gather the start time and end time for a sub-event. In this example, the sub-event is a point in a game. It should be noted that the invention is not limited to the type of sub-events (e.g., in this case, points played, with the outcome being point won or point lost). The invention can be applicable any sub-event that leads to a measurable, definable, observable, and/or quantifiable outcome. For example, a shot or possession in a basketball game (e.g., make a shot vs miss a shot) or soccer match (e.g., made penalty kick vs missed penalty kick), a golf shot (make the putt vs miss the put; hit the drive straight vs hit the drive left/right), a forehand in a tennis match (make the shot vs miss the shot), a possession or throw in a football game (e.g., first play of a drive), and the like. In these examples, the start time and end time would be related to a tunable period of time prior to, and after, the sub-event occurring that led to a measurable, definable, observable, and/or quantifiable outcome.

Step 4: Gather the R-R Intervals data from one or more sensors for the targeted individual from the start time to end time obtained above in Step 3.

Step 5: Calculate CURRENT_RMSSD=RMSSD value from R-R Intervals data obtained in Step 4.

Step 6: Calculate SDL_RMSSD=(CURRENT_RMSSD−BASELINE_RMSSD)/BASELINE_RMSSD. In a variation, this output can be labeled “HRV Difference.”

Step 7: Calculate SDL_RMSSD for point A and SDL_RMSSD for point B where A and B are successive points played. In a variation, A and B may not be successive points but intermittent points, other successive or intermittent events (e.g., all shots on a single golf hole, or only drives in a golf round), or the like. This can be a tunable parameter.

Step 8: Calculate DIFFERENCE_IN_RMSSD=(SDL_RMSSD at point B)−(SDL_RMSSD at point A) where point A and B are defined in Step 7. In a variation, this output can be labeled “Variability Indicator.”

Step 9: Repeat Step 8 for all points in a game. In some variations, Step 9 can be for select points or other select events (e.g., select shots, holes, plays, possessions). This can be a tunable parameter.

Step 10: Calculate SPIKE_RMSSD as:

```
IF DIFFERENCE_IN_RMSSD >= SDL_SPIKE_MAGNITUDE_THRESHOLD
  SPIKE_RMSSD = 1
OTHERWISE
  SPIKE RMSSD = 0
```

(SDL_SPIKE_MAGNITUDE_THRESHOLD is a variable with value >0). Note that this threshold can be a tunable parameter (e.g., tunable by the system, by one or more AI techniques, and the like).

Step 11: Calculate SPIKE_RMSSD_COUNT=Number of SPIKE_RMSSD per game for the targeted individual. Note that the output of Step 10 and/or Step 11 can be considered a Variability Indicator.

Step 12: Gather game outcome as a win or loss by the targeted individual.

Step 13: Initialize GAME_WON_COUNT=0 if not initialized already and increment by 1 if the game outcome was a win in Step 12.

Step 14: Repeat Steps 1-13 for all points in a game. In some variations, Step 14 can be for select points, select events (e.g., select shots, holes, plays, possessions), or the like. This can be a tunable parameter.

Step 15: Calculate GAME_WON_PERCENTAGE as percentage of GAME_WON_COUNT out of Total Games played

Step 16: Calculate SDL_PERSONALIZED_SPIKE_COUNT=SPIKE_RMSSD_COUNT with highest value of GAME_WON_PERCENTAGE or GAME_LOST_PERCENTAGE

(e.g. For Player A
when SPIKE_RMSSD_COUNT=2 Win Percentage=55
when SPIKE_RMSSD_COUNT=3 Win Percentage=60
when SPIKE_RMSSD_COUNT=4 Win Percentage=75
when SPIKE_RMSSD_COUNT=5 Win Percentage=90
then Player A's SDL_PERSONALIZED_SPIKE_COUNT=5

### For Player B

when SPIKE_RMSSD_COUNT=2 Win Percentage=55
when SPIKE_RMSSD_COUNT=3 Win Percentage=80
when SPIKE_RMSSD_COUNT=4 Win Percentage=60
when SPIKE_RMSSD_COUNT=5 Win Percentage=65
then Player B's SDL_PERSONALIZED_SPIKE_COUNT=3

### For Player C

when SPIKE_RMSSD_COUNT=2 Win Percentage=50
when SPIKE_RMSSD_COUNT=3 Win Percentage=45
when SPIKE_RMSSD_COUNT=4 Win Percentage=40
when SPIKE_RMSSD_COUNT=5 Win Percentage=30
then Player As SDL_PERSONALIZED_SPIKE_COUNT=−5

### For Player D

when SPIKE_RMSSD_COUNT=2 Win Percentage=55
when SPIKE_RMSSD_COUNT=3 Win Percentage=35
when SPIKE_RMSSD_COUNT=4 Win Percentage=40
when SPIKE_RMSSD_COUNT=5 Win Percentage=40
then Player B's SDL_PERSONALIZED_SPIKE_COUNT=−3)

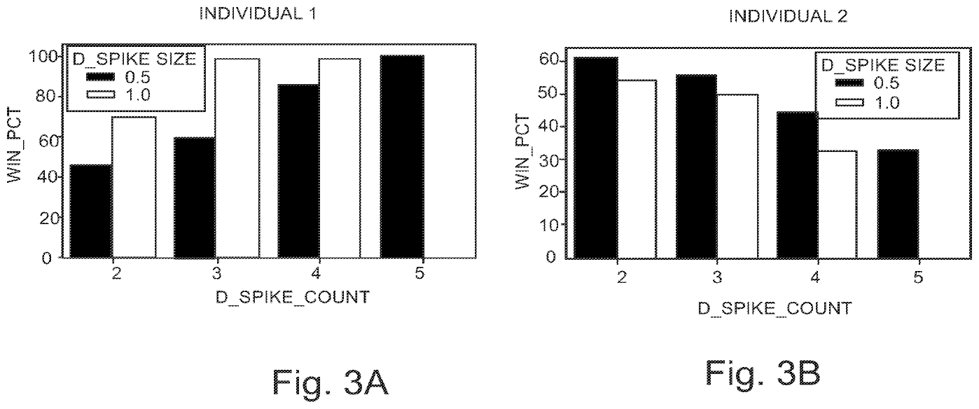

Such analysis can provide baseline information related to the number of spike counts and the corresponding win percentage for each individual. Note that the number of spikes can be a tunable parameter. FIG. 3 illustrates example outputs from the described steps.

Any output from any one or more calculations in Steps 1-16 can be classified as one or more reference insights. The R-R Intervals data, its derivatives, and any outputs from any one or more calculations in Steps 1-16 can be included as part of the reference animal data. Such derivatives and outputs are displayed in FIG. 1 as reference insight 76. In a refinement, one or more reference insights can include any portion of reference animal data. In another refinement, any contextual data associated with the R-R Intervals data or its derivatives is included as part of the reference animal data. In some variations, upon executing Steps 1-16, the computing subsystem executes another series of steps in order to calculate the probability of the targeted individual winning or losing an event or sub-event (e.g., a game within a live sports match) in real-time or near real-time. In other variations, upon executing Steps 1-16, the computing subsystem executes another series of steps in order to create, modify, or enhance or more predictive indicators related to the targeted individual in real-time or near real-time during an event or sub-event.

In another embodiment for a method for generating dynamic real-time predictions using heart rate variability, a computing subsystem creates, modifies, or enhances one or more probabilities of win (or loss) based upon the gathered HRV data from the targeted individual. Using reference animal data as a baseline for each targeted individual, the computing subsystem executes the following series of steps:

Step 1: Gather the R-R Intervals data from one or more sensors for the targeted individual prior to the start of an event for n period of time (e.g., seconds, minutes, hours or longer). Time n is a tunable parameter.

Step 2: Calculate BASELINE_RMSSD=RMSSD value from the R-R Intervals data obtained above in Step 1. FIG. 2 shows the R-R intervals labeled IBI₁=(R-R)₁, IBI₂=(R-R)₂, IBI₃=(R-R)3 . . . (R-R)ᵢ represented the interval between two adjacent QRS peaks. The RMSSD is looking for the successive difference between the intervals meaning: IBI₁-IBI₂→(R-R)₁-(R-R)₂; IBI₂-IBI₃→(R-R)₂-(R-R)₃; and the like. In one variation, RMSSD can be calculated using the following formula:

$$RMSSD = \sqrt{\frac{1}{N - 1} ( {\sum }_{i - 1}^{N - 1} {( {( R - R )}_{i + 1} - {( R - R )}_{i} )}^{2} )}$$

where N=number of R-R interval terms.

Step 3: Gather the start time and end time for a sub-event: a point in a game. It should be noted that the invention is not limited to sub-events consisting of points played. The invention can be applicable to any singular event that leads to a measurable, definable, observable, and/or quantifiable outcome. For example, a shot or possession in a basketball game (e.g., make a shot vs miss a shot), a golf shot (make the putt vs miss the put; hit the drive straight vs hit the drive left/right), a forehand in a tennis match (make the shot vs miss the shot), a possession in a football game (e.g., the first play of a drive), and the like. In these examples, the start time and end time would be related to a tunable period of time prior to, and after, the sub-event occurring that led to a measurable, definable, observable, and/or quantifiable outcome.

Step 4: Gather the R-R Intervals data from one or more sensors for the targeted individual from start time to end time obtained above in Step 3.

Step 5: Calculate CURRENT_RMSSD=RMSSD value from R-R Intervals data obtained in Step 4.

Step 6: Calculate SDL_RMSSD=(CURRENT_RMSSD−BASELINE_RMSSD)/BASELINE_RMSSD. In a variation, this can be labeled “HRV Difference.”

Step 7: Calculate SDL_RMSSD for point A and SDL_RMSSD for point B where A and B are successive points played. In a variation, A and B may not be successive points but intermittent points, other successive or intermittent events (e.g., only three-point shots in a basketball game or throws over 50 yards in a football game), or the like. This can be a tunable parameter.

Step 8: Calculate DIFFERENCE_IN_RMSSD=(SDL_RMSSD at point B)−(SDL_RMSSD at point A) where point A and B are defined in Step 7. In a variation, this output can be labeled “Variability Indicator.”

Step 9: Repeat Step 8 for all points in a game. In some variations, Step 9 can be for select points or select events (e.g., select shots, holes, plays, possessions). This can be a tunable parameter.

Step 10: Calculate SPIKE_RMSSD (difference in the RMSSD between two successive points) as:

```
IF DIFFERENCE_IN_RMSSD >= SDL_SPIKE_MAGNITUDE_THRESHOLD
  SPIKE_RMSSD = 1
OTHERWISE
  SPIKE_RMSSD = 0
```

(where SDL_SPIKE_MAGNITUDE_THRESHOLD is a variable with value >0). Note that this threshold can be a tunable parameter.

Step 11: Calculate SPIKE_RMSSD_COUNT=Number of SPIKE_RMSSD per game for the targeted individual (e.g., how many spikes per game occurred). Note that the output of step 10 and/or step 11 can be considered a Variability Indicator.

### Step 12:

If ((SPIKE_RMSSD_COUNT > ABSOLUTE VALUE OF
(SDL_PERSONALIZED_SPIKE_COUNT)) and (SDL_PERSONALIZED_SPIKE_COUNT > 0))
then SDL_WIN_PROBABILITY is greater than 50%;
Else If ((SPIKE_RMSSD_COUNT > ABSOLUTE VALUE OF
(SDL_PERSONALIZED_SPIKE_COUNT)) and (SDL_PERSONALIZED_SPIKE_COUNT < 0))
then SDL_WIN_PROBABILITY is less than 50%

Step 13: Take the output from Step 12 and if contextual data is gathered, use contextual data to modify or enhance SDL_WIN_PROBABILITY by using one or more weighted values for each contextual data, the one or more values being weighted by one or more Artificial Intelligence techniques. In a refinement, the output of the one or more AI techniques is the weight assigned to the one or more contextual data features (e.g., the augmented data set has one or more features in addition to what is described herein and has a weight/importance that contributes to the win probability/prediction).

Step 14: Gather and record the outcome of the event or sub-event against the SDL_WIN_PROBABILITY as generated in Step 13 and associate each gathered contextual data with the recorded outcome.

Step 15: Provide data gathered and associated from Step 14 to one or more Artificial Intelligence models to train the one or more Artificial Intelligence-based models related to the impact of each gathered contextual data on the associated outcome and SDL_WIN_PROBABILITY.

Step 16: Create, modify or enhance one or more weighted values from Step 13 based upon the one or more outputs of Step 15.

Any output from any one or more calculations in Steps 1-11 and 14-16 can be classified as one or more primary insights. The R-R Intervals data, its derivatives, and any outputs from any one or more calculations in Steps 1-11 and 14-16 are labeled as primary insight 72 in FIG. 1. Any predictions, probabilities, forecasts, assessments, projections, recommendations, or possibilities derived from Steps 12-13 are displayed in FIG. 1 as predictive indicator 74. In a refinement, contextual data can be used in conjunction with heart rate variability or HRV-derived data to modify or enhance at least one primary insight or one or more predictive indicators for one or more events or sub-events. One or more Artificial Intelligence techniques (e.g., AI-based models) can be utilized to modify or enhance at least one primary insight or one or more predictive indicators for the one or more events or sub-events.

In a variation, the one or more steps in the one or more embodiments can be executed on a single computing device or across multiple computing devices. In another variation, the one or more steps in the one or more embodiments can be executed on a single computing device, on multiple computing devices, via one or more sensors, or a combination thereof. In other variations, one or more steps from each of the embodiments can be combined. For example, HRV data gathered in real-time for the purposes of creating one or more probabilities related to win/loss may then be synced with outcome data and included as part of the reference animal data. In some variations, the embodiments operate together as part of a system. In other variations, the embodiments operate independently of each other, which can be part of the same system or different systems.

Still referring to FIG. 1, computing subsystem 22 can gather animal data 14 from source 12 via one or more communication links either wirelessly, via one or more wired connections, or a combination thereof. Computing subsystem 22 may also include a transmission subsystem 24 that includes one or more hardware and software components that enable electronic communication with one or more sources 12 of animal data 14. In this regard, computing subsystem 22 receives and collects the animal data 14 through transmission subsystem 24. Typically, transmission subsystem 24 includes a transmitter and a receiver, or a combination thereof (e.g., transceiver). Transmission subsystem 24 can include one or more receivers, transmitters and/or transceivers having a single antenna or multiple antennas (e.g., which can be configured as part of a mesh network). In some variations, the transmission subsystem can include one or more receivers, transmitters, transceivers, and/or supporting components (e.g., dongle) that utilize a single antenna or multiple antennas, which can be configured as part of a mesh network and/or utilized as a part of an antenna array. The transmission subsystem and/or its one or more components can be housed within the one or more computing devices or can be external to the computing device (e.g., a dongle connected to the computing device which includes one or more hardware and/or software components that facilitate wireless communication and is part of the transmission subsystem). In a refinement, one or more components of the transmission subsystem and/or one or more of its components are integral to, included within, or attached to, the one or more sensors 18.

In a variation, transmission subsystem 24 can communicate electronically with the one or more sensors 18 from the one or more targeted individuals 16 using one or more wireless methods of communication via one or more communication links. In a variation, transmission subsystem 24 enables the one or more source sensors 18 to transmit data wirelessly via one or more transmission (e.g., communication) protocols. In this regard, prediction system 10 can utilize any number of communication protocols and conventional wireless networks, including any combination thereof (e.g., BLE and LoRa to create hybrid connectivity for combined short and long-range communication), to communicate with one or more sensors 18 including, but not limited to, Bluetooth Low Energy (BLE), ZigBee, cellular networks, LoRa/LPWAN, NFC, ultra-wideband, Ant+, WiFi, and the like. The present invention is not limited to any type of technology or electronic communication links (e.g., radio signals) the one or more sensors 18 or any other computing device utilized to transmit and/or receive signals. Advantageously, the transmission subsystem enables the one or more sensors 18 to transmit data wirelessly for real-time or near real-time communication. In this context, near real-time means that the transmission is not purposely delayed except for necessary processing by the sensor and any other computing device taking one or more actions on, with, or related to the data. In another variation, one or more apparatus with one or more onboarded computing devices (e.g., such as an aerial apparatus like an unmanned aerial vehicle or other remote computing devices) may act as a transmission subsystem to collect and distribute animal data from one or more sensors or other information from one or more targeted subjects or groups of targeted subjects. In a refinement, the one or more apparatus may have one or more sensors attached, or integrated, as part of the apparatus to collect animal data. In another variation, computing subsystem 22 can also gather animal data 14 from one or more source sensors 18 directly via a wired connection. In a refinement, transmission subsystem 24 can be comprised of multiple transmission subsystems 24.

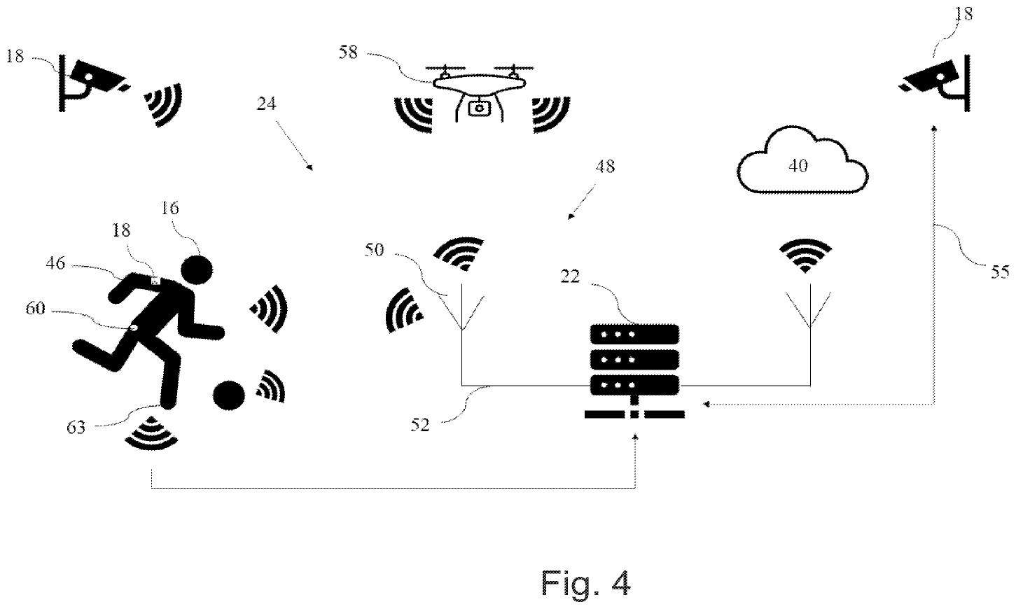

FIG. 4 depicts variations of transmission subsystem 24. Computing subsystem 22 receives a signal from sensor 18 gathering animal data from targeted individual 16. Sensor 18 can include an integral transmitter, receiver, or transceiver 46. Advantageously, the transmission subsystem enables the one or more source sensors to transmit data wirelessly for real-time or near real-time communication. In addition, the transmission subsystem can communicate with the one or more source sensors utilizing one or more transmission protocols. The present invention is not limited by the technologies that sensors 18 use to transmit and/or receive signals. Receiver 48 receives the signal from transmitter 46. In a variation, one or more sensors 18 is also represented by one or more optical sensors 18 which is not attached to targeted individual 16 and connected to computing subsystem 22 via a wired connection 55. In another variation, optical sensor 18 is also depicted as being operable to communicate with computing subsystem 22, cloud 40, unmanned aerial vehicle sensor 58, or a combination thereof wirelessly. In a refinement, optical sensor 18 can communicate with another one or more sensors 18 (e.g., including one or more other optical sensors 18).

In the depicted variation in FIG. 4, receiver 48 includes antenna 50. Antenna 50 may also include one or more supporting components depending on the configuration (e.g., a dongle such as a BlueTooth transceiver). In a refinement, antenna 50 and/or one or more supporting components thereof can be integral to or attached to computing subsystem 22 or external to computing subsystem 22 (e.g., located at a distance from computing subsystem 22—for example, 100 feet, 1000 feet, or more). In this situation, antenna 50 can be connected to computing subsystem 22 via wired connection line 52. In some variations, the connection between antenna 50 and computing subsystem 22 can be wireless. In a refinement, antenna 50 can include a plurality of antennas. In another refinement, the transmission subsystem, or the one or more components related to the transmission subsystem (e.g., antenna 50), can be wearable and can be affixed to, in contact with, or integrated with, the subject either directly or via one or more intermediaries (e.g., clothing, equipment, and the like). The transmission subsystems, or components of the transmission subsystem, may also be mobile or personal to the one or more individuals. In another refinement, transmission subsystem 24 includes an on or in-body transceiver 60 (“on-body transceiver”) that optionally acts as another sensor or is optionally integrated within a sensor. On-body transceiver 60 can be operable to communicate with the one or more sensors 18 on a target subject or across one or more target subjects, and may itself track one or more types of biological data (e.g., positional or location data). In this regard, on-body transceiver 60 can be a computing device. In another refinement, on-body transceiver operates as sensor 18. In another refinement, the on-body transceiver is affixed to, integrated with, or in contact with, a subject's body, skin, hair, vital organ, muscle, skeletal system, eyeball, clothing, object, or other apparatus on a subject. Advantageously, the on-body transceiver collects the one or more data streams in real-time or near real-time from one or more sensors gathering data from the subject (e.g., on a subject's body), communicating with each sensor using a transmission protocol of that particular sensor. In a refinement, the on-body transceiver is operable to utilize multiple transmission protocols to communicate with multiple sensors. The on-body transceiver may also act as a data collection hub from one or more sensors 18 for each subject or subset of subjects. In a refinement, the on-body transceiver can take on one or more functionalities related to transmission subsystem 24, computing subsystem 22, one or more sensors 18, or a combination thereof. In another refinement, on-body transceiver 60 can enhance one or more transmission-related qualities related to the gathering of animal data 14 by computing subsystem 22 (e.g., increase speed, reducing latency, and the like). In another refinement, the on-body transceiver can include logic that enables the on-body transceiver to perform at least one action on the animal data from the group consisting of: collecting, normalizing, time stamping, aggregating, processing, transforming, tagging, storing, manipulating, denoising, productizing, enhancing, organizing, categorizing, visualizing, analyzing, summarizing, replicating, synthesizing, anonymizing, synchronizing, or distributing the animal data. Characteristically, the on-body transceiver can be operable to communicate with one or more computing devices (e.g., computing subsystem 22, cloud 40). In some variations, such communication can be two-way communication that enables one or more computing devices to send one or more commands to the on-body transceiver or take one or more actions via the on-body transceiver (e.g., a computing device can select one or more parameters related to the animal data via the on-body transceiver, or select the types of animal data gathered from (or by) the on-body transceiver). For example, computing subsystem 22 may want to request the on-body transceiver to send one or more types of animal data to computing subsystem 22 while sending other types of animal data to another computing device (e.g., cloud 40). In another example, computing subsystem 22 may want the on-body transceiver to provide data to the computing device at 1 Hz while the on-body transceiver is receiving data from the one or more sensors at 1000 Hz. In some variations, the on-body transceiver can send any collected and selected data to n number of endpoints in real-time or near real-time, while enabling any data not selected to be stored on the transceiver for download at a later time or sent to another computing device (e.g., cloud 40) on a continuously or intermittently (which can be a tunable parameter). In addition, the on-body transceiver's capabilities enables data that may, for example, be sampled at high frequency rates (e.g., 250-1000 Hz or more) to be summarized and sent in summarized or processed form (e.g., the data processed and/or summarized at 1 Hz) to accommodate any number of use cases or constraints (e.g., limited bandwidth).

In another variation, transmission subsystem 24 includes an aerial transceiver 58 for continuous streaming and/or intermittent communication from the one or more sensors located on one or more target subjects or objects. Characteristically, aerial transceiver 58 may also include one or more sensors (e.g., optical sensors, infrared sensors) that capture one or more types of animal data (e.g., location data, physiological data, biomechanical data, and the like). Examples of aerial transceiver 58 include, but are not limited to, one or more communications satellites or unmanned aerial vehicles with attached transceivers (e.g., high-altitude pseudo satellites, drones). Additional details related to an unmanned aerial vehicle-based animal data collection and distribution system are disclosed in U.S. patent Ser. No. 16/977,570 filed Sep. 2, 2020, with a priority date of Jul. 19, 2019; the entire disclosure of which is hereby incorporated by reference. In another variation, transmission subsystem 24 includes a transceiver 63 embedded or integrated as part of a floor or ground (including a field). In a refinement, the transmission of data occurs via direct or indirect contact with a surface (e.g., in the event the sensor or transceiver is located on or near the bottom of the shoe).

In some variations, the communication distance between a sensor and a receiver of the sensor signal can be elongated by transmission subsystem 24 to enable real-time or near real-time communication over longer distances, thereby extending a range limitation of the one or more sensors and their corresponding one or more transmission protocols. Furthermore, variations of the transmission subsystem enable real-time or near real-time streaming in environments where potential radio frequency (RF) interference (e.g., noise) or bandwidth issues occur. In a refinement, the computing subsystem 22 synchronizes the communication between the one or more sensors and the computing subsystem 22. In another refinement, computing device 22 synchronizes the one or more data streams, which can occur in real-time or near real-time, derived from the one or more sensors that are communicating with computing subsystem 22 utilizing at least a portion of non-animal data (e.g., metadata such as timestamps). In another refinement, the computing subsystem 22 sends at least a portion of the animal data to one or more other computing devices (e.g., another computing device within the system or another system) or stores the animal data for later use. In another refinement, the system may provide a real-time or near real-time backup mechanism for incoming data from the one or more source sensors with minimal effect on the real-time or near real-time transmission.

Referring to FIG. 1, computing subsystem 22 gathers (e.g., receives, collects) animal data 14 from one or more sensors 18, one or more computing devices 26, one or more computing devices 25, one or more clouds 40, or a combination thereof. Computing subsystem 22 includes an operating system that coordinates interactions between one or more types of hardware and software. Computing subsystem 22 can be comprised of a single computing device or multiple computing devices as part of one or more systems. A system can be one or more sets of one or more interrelated or interacting components which work together towards achieving one or more common goals or producing one or more desired outputs. The one or more components of a system can include one or more applications, frameworks, platforms or other subsystems, which can be integral to the system or separate from the system but part of a network or multiple networks linked with the system and operable to achieve the one or more common goals or produce the one or more desired outputs. In a refinement, Computing subsystem 22 incudes a plurality of computing subsystems 22. Computing subsystem 22 may also include one or more network connections, such as an internet connection or cellular network connection, which may include hardware and software aspects, or pre-loaded hardware and software aspects that do not necessitate an internet connection. Computing subsystem 22 can be operable for wired communication, wireless communication, or a combination thereof.

Computing subsystem 22 is operable to receive the animal data or groups of animal data from a single targeted individual or multiple targeted individuals as raw or processed (e.g., manipulated) animal data. In a refinement, computing subsystem 22 is operable to receive a single type of animal data (e.g., heart rate data) and/or multiple types (e.g., including groups/data sets) of animal data (e.g., raw analog front end data, heart rate data, muscle activity data, accelerometer data, hydration data, biological fluid data) from a single sensor and/or multiple sensors derived from a single targeted individual and/or multiple targeted individuals.

Computing subsystem 22 can also gather contextual data 13 from one or more sensors, one or more programs operating via computing subsystem 22 (e.g., if the contextual data is manually entered or gathered), one or more other computing devices which operate one or more programs that gather data, or a combination thereof. Contextual data 13 can include any set of data that describes and provides information about other data, including data that provides context for other data (e.g., the activity or event a targeted individual is engaged in while the animal data is collected, the outcome of the activity the targeted individual is engaged in, the one or more characteristics/attributes of the targeted individual, animal data that provides context for other animal data, and the like). Contextual data can also include the one or more variables that can affect the one or more animal data readings (e.g., cause one or more changes or variations in the animal data, including animal data, non-animal data, or a combination thereof), the one or more event outcomes, or a combination thereof. Contextual data can be animal data, non-animal data, or a combination thereof. In many variations, animal data 14 collected by computing subsystem 22 can include or have attached thereto contextual data such as individualized metadata, which may include one or more characteristics directly or indirectly related to the animal data, including characteristics related to the one or more sensors, (e.g., identity of the sensor, sensor type, sensor brand, sensing type, sensor model, firmware information, sensor positioning on or related to a subject, sensor operating parameters, sensor configurations, sensor properties, sampling rate, mode of operation, data range, gain, battery life, shelf life/number of times the sensor has been used, timestamps, and the like), characteristics/attributes of the one or more targeted individuals, origination of the animal data (e.g., event, activity, or situation in which the animal data was collected, duration of data collection period, quality of data, when the data was collected), type of animal data, source computing device of the animal data, location, data format, algorithms used, quality of the animal data, quality of data, size/volume/quantity of the data, latency information, speed at which the animal data is provided, environmental condition, bodily condition, and the like. Metadata can also be associated with the animal data after it is collected. Metadata can include non-animal data, animal data, or a combination thereof. Metadata can also include one or more characteristics/attributes directly or indirectly related to the one or more targeted individuals. Contextual data 13 can be metadata associated with the animal data, the one or more targeted subjects, the one or more sensors, the one or more events associated with the one or more targeted subjects, or a combination thereof. In a refinement, contextual data 13 is metadata associated with animal data. In another refinement, contextual data 13 is inclusive of metadata associated with animal data. In another refinement, contextual data 13 can be other information gathered that that provides context to the gathered animal data but not classified as metadata. In another refinement, contextual data is data derived from one or more Artificial Intelligence techniques that provides context to other data. Upon being collected by computing subsystem 22 or a computing device in communication with computing subsystem 22 (e.g., cloud server 40), contextual data 13 can be assigned as reference contextual data 113.

In a variation, the system can be configured to create, modify, or enhance one or more tags based upon the contextual data (e.g., contextual data 13) related to the animal data (e.g., including contextual information and other metadata), the one or more targeted subjects, the one or more sensors, the one or more events associated with the one or more targeted subjects, or a combination thereof. Tags (e.g., including classifications or groups that a targeted subject can be assigned to such as basketball team, individuals with a specific type of disease or blood type, and the like, or classifications or groups that medical conditions associated with the targeted individual can be assigned to) can be identifiers for data, can support the indexing and search process for one or more computing devices or data acquirers (e.g., tags can simplify the search process as one or more searchable tags), and can be based on data collection processes, practices, quality, or associations, as well as targeted individual characteristics. A characteristic may include specific personal attributes or characteristics of the one or more subjects or groups of subjects from which the animal data is derived (e.g., name, weight, height, corresponding identification or reference number, medical history, personal history, health history, medical condition, biological response, and the like), as well as information related to the animal data, its associated metadata, and the one or more sources of the animal data such as sensor type, sensor model, sensor brand, firmware information, sensor positioning, timestamps, sensor properties, classifications, specific sensor configurations, operating parameters (e.g., sampling rate, mode, gain, sensing type), mode of operation, data range, location, data format, type of data, algorithms used, quality of the data, size/volume/quantity of the data, analytics applied to the animal data, data value (e.g., actual, perceived, future, expected), when the data was collected, associated organization, associated activity, associated event (e.g., simulated, real world), latency information (e.g., speed at which the data is provided), environmental condition (e.g. if the data was collected in a dangerous condition/environment, rare or desired condition/environment, and the like), bodily condition (e.g., if a person has stage 4 pancreatic cancer or other bodily condition), context (e.g., data includes a monumental moment/occasion, such as achievement of a threshold or milestone within the data collection period may make the data more valuable), duration of data collection period, quality of data (e.g., a rating or other indices applied to the data, completeness of a data set, noise levels within a data set, data format), monetary considerations (e.g., cost to create or acquire, clean, and/or structure the animal data; value assigned to the data), non-monetary considerations (e.g., how much effort and time it took to create or acquire the data), and the like. It should be appreciated that any single characteristic related to animal data (e.g., including any characteristic related to the data, the one or more sensors, the metadata, the one or more targeted subjects, the one or more medical conditions, the one or more biological responses, and the like) can be assigned or associated with one or more tags as contextual data. Characteristically, the one or more tags associated with the animal data can contribute to creating, modifying, or enhancing an associated value (e.g., monetary, non-monetary) for the animal data. In a refinement, one or more Artificial Intelligence techniques (e.g., Machine Learning, one or more neural networks, Statistical Learning) are utilized to assign, create, modify, remove, or a combination thereof, one or more tags related to the animal data (e.g., including its metadata), the one or more targeted subjects, the one or more source sensors, the one or more events associated with the one or more targeted subjects, or a combination thereof. In another refinement, the one or more computing devices verify the one or more tags associated with the targeted individual, the one or more source sensors, the animal data (e.g., including its metadata), the one or more events associated with the one or more targeted subjects, or a combination thereof. In another refinement, one or more tags are created, modified, or enhanced for reference animal data 114 based upon reference contextual data 113.

Examples of contextual data derived from or related to a targeted individual's one or more characteristics/attributes can include but are not limited to, name, age, weight, height, birth date, race, eye color, skin color, hair color (if any), country of origin, country of birth (if different), area of origin, ethnicity, current residence, addresses, phone number, reference identification (e.g., social security number, national ID number, digital identification), gender of the targeted individual from which the animal data originated, data quality assessment, and the like. In a refinement, the targeted individual's characteristics/attributes can also include information (e.g., animal data) gathered from medication history, medical history, medical records, health records, genetic-derived data, genomic-derived data, (e.g., including information related to one or more medical conditions, traits, health risks, inherited conditions, drug responses, DNA sequences, protein sequences, and structures), biological fluid-derived data (e.g., blood type), drug/prescription records, allergies, family history, health history (including mental health history), manually-inputted personal data, physical shape (e.g. body shape), historical personal data, training regimen, nutritional history, and the like. The targeted individual's one or more attributes can also include one or more activities the targeted individual is engaged in while the animal data is collected, one or more associated groups (e.g., if the individual is part of a sports team, or assigned to a classification based on one or more medical conditions), one or more habits (e.g., tobacco use, alcohol consumption, exercise habits, nutritional diet, the like), education records, criminal records, financial information (e.g., bank records, such as bank account instructions, checking account numbers, savings account numbers, credit score, net worth, transactional data), social data (e.g., social media accounts, social media history, records, internet search data, social media profiles, metaverse profiles, metaverse activities/history), employment history, marital history, relatives or kin history (in the case the targeted subject has one or more children parents, siblings, and the like), relatives or kin medical history, relatives or kin health history, manually inputted personal data (e.g., one or more locations where a targeted individual has lived, emotional feelings, mental health data, preferences), historical personal data, and/or any other individual-generated data (e.g., including data about or related to the individual). In a refinement, one or more characteristics/attributes associated with another one or more subjects can be associated with one or more targeted individuals as metadata. For example, in the event the targeted individual has children, the subject's (i.e., child's) health condition can be associated with the one or more targeted individuals as a characteristic associated with the one or more targeted individuals' data (e.g., if the child is sick, the parent can be under considerable stress or have deteriorating mental health which may impact their animal data). In another example, the one or more characteristics/attributes of the targeted individual's avatar or representation in a virtual environment, video game, or other simulation (e.g., including their actions, experiences, conditions, preferences, habits, and the like) can be associated with the targeted individual as metadata and can be included as part of the targeted individual's animal data. In another refinement, animal data is inclusive of the targeted individual's one or more characteristics/attributes (i.e., the one or more characteristics/attributes can be categorized as animal data). In another refinement, at least a portion of gathered data can be classified as both animal data and metadata. In another refinement, the system may associate metadata with one or more types of animal data prior to its collection (e.g., the system may collect one or more attributes related to the targeted individual prior to the system collecting animal data and associate the one or more attributes in the targeted individual's profile to the one or more types of animal data prior to its collection).

Examples of contextual data in the context of a sporting event can also include, but are not limited to, event data such as traditional sports statistics collected during an event (e.g., any given outcome data, including game score, set score, match score, individual quarter score, halftime score, final score, points, rebounds, assists, shots, goals, pass accuracy, touchdowns, minutes played, and other similar traditional statistics), in-game data (e.g., whether the player is on-court vs off-court, whether the player is playing offense vs defense, whether the player has the ball vs not having the ball, the player's location on the court/field at any given time, specific on-court/field movements at any given time, who the player is guarding on defense, who is guarding the player on offense, ball speed, ball location, exit velocity, spin rate, launch angle), streaks (e.g., consecutive points won vs lost; consecutive matches won vs lost; consecutive shots made vs missed), competition (e.g., men, women, other), round of competition (e.g., quarterfinal, finals), matchup (e.g., player A vs. player B; team A vs team B), opponent information, type of event (e.g., exhibition vs real competition), date, time, location (e.g., specific court, arena, field, and the like), crowd size, crowd noise levels, prize money amount, number of years associated with the event (e.g., number of years a player has been playing within a specific league or with a specific team), ranking or standing/seeding, the type of sport, level of sport (professional vs amateur), career statistics (e.g., in the case of individual athletes in racquet sports as an example, number of: tournaments played, titles, matches played, matches won, matches lost, games played, games won, games lost, sets, sets won, sets lost, points played, points won, points lost, retirements, and the like), points won vs. points played, games (e.g., sets) won vs. games played, matches won vs. matches played, any given round rate (e.g., finals win/loss rate or semi-finals win/loss rate; number of times a player makes any given round in any given tournament (e.g., number of times a player makes the semifinals in any given tournament, which may on a yearly or career basis), title win rate (e.g., how many times the player has won this year or any given year or over a career; how many times a player has won that particular tournament), match retirement history, court surface (e.g., hard court vs clay court), and the like. Contextual data can also include information such as historical animal data/reference animal data (e.g., outcomes that happened which are cross referenced with what was happening with the athlete's body and factors surrounding it such as their heart rate and HRV data, body temperature data, distance covered/run data for a given point/game/match, positional data, biological fluid readings, hydration levels, muscle fatigue data, respiration rate data, any relevant baseline data, an athlete's biological data sets against any given team, who the player guarded in any given game, who guarded the player in any given game, the player's biological readings guarding any given player, the player's biological readings being guarded by any given player, minutes played, court/ground surface, the player's biological readings playing against any given offense or defense, minutes played, on-court locations and movements for any given game, other in-game data), comparative data to similar and dissimilar players in similar and dissimilar situations (e.g., other player stats when guarding or being guarded by a specific player, playing against a specific team) injury data (e.g., including injury history), recovery data (e.g., sleep data, rehabilitation data), training data (e.g., how the player performed in training in the days or weeks leading up to a game), nutrition data, a player's self-assessment data (e.g., how they're feeling physically, mentally, or emotionally), nutritional data, mental health data, and the like. It can also include information such as country of origin, height, weight, dominant hand or handedness (e.g., right hand dominant vs left hand dominant), residence, equipment manufacturer, coach, race, nationality, habits, activities, genomic information, genetic information, medical history, family history, medication history, and the like. Contextual information can also be scenario-specific. For example, in the sport of tennis, contextual information can be related to when a player is winning 2-0 or 2-1 in sets or losing 1-2 or 0-2 in sets, or time of day the player is playing, or the specific weather conditions the game is played in. Contextual information can also be related to head-to-head match ups. In the sport of squash for example, head-to-head information can be related to the number of head-to-head matches, games, number of times a player has been in a specific scenario vs the other player (e.g., in terms of game score: 3-0, 3-1, 3-2, 2-3, 1-3, 0-3, 2-0, 2-1, 1-2, 0-2, or retired). Contextual information can also include how that player has performed in that particular tournament (e.g., matches played, matches won, games played, games won/lost, sets played, sets won/lost, court time per match, total court time, previous scores and opponents, and the like). Characteristically, the system can be configured to evaluate a single type of data or a plurality of data (e.g., data types, data sets) simultaneously. For example, in the context of a sport like tennis, the system may evaluate multiple sources of data and data types simultaneously utilizing one or more Artificial Intelligence techniques such as sensor-based animal data readings (e.g., positional data, location data, distance run, physiological data readings, biological fluid data readings, biomechanical movement data), non-animal data sensor data (e.g., humidity, elevation, and temperature for current conditions; humidity, elevation, and temperature for previous match conditions), length of points, player positioning on court, opponent, opponent's performance in specific environmental conditions, winning percentage against opponent, winning % against opponent in similar environmental conditions, current match statistics, historical match statistics based on performance trends in the match, head-to-head win/loss ratio, previous win/loss record, ranking, a player's performance in the tournament in previous years, a player's performance on court surface (e.g., grass, hard court, clay), length of a player's previous matches, current match status of a tennis player (e.g., athlete A is in Game 3 of Set 1 and is losing 5-2) and their historical data in the context of the current match status (e.g., all of athlete A match results when athlete A is in Game 3 of Set 1 and is losing 5-2, first serve percentage in second sets after playing n number of minutes, unforced errors percentage on the backhand side after hitting three n topspin backhands), and the like. In a refinement, any contextual data related to an event (either directly or indirectly) can be categorized as event data for (or associated with) the event. In another refinement, contextual data is inclusive of event data. In another refinement, event data is comprised of any contextual data associated either directly or indirectly with the event. In another refinement, event data includes at least a portion of contextual data.

In another refinement, contextual data (e.g., contextual information) can also include an exponential moving average of a player's “momentum” (e.g., which can be quantified by the number of consecutive or non-consecutive occurrences such as the number of consecutive or non-consecutive occurrences points won or lost in any given game, the number of consecutive or non-consecutive game wins vs consecutive game losses, the number of consecutive or non-consecutive match wins vs consecutive or non-consecutive match losses, the number of consecutive or non-consecutive matches won vs lost against any given player, some or all of which can be weighted and/or continuously or intermittently created or modified via a rolling window method and represented as a number, metric or other indicator; the number of consecutive points, games or matches won vs lost on any given surface, in any given venue, using any particular equipment, in any given environmental conditions, exhibiting any given animal data-based traits; the number of consecutive or non-consecutive occurrences of the same or materially similar nature for any given event; and the like). In some variations, “momentum” can be an indicator, score, or other quantifiable numeric or non-numeric metric or indices based upon the number of consecutive or non-consecutive occurrences of the same or materially similar nature for any given event (e.g., an indicator quantifying “momentum” created based upon a player winning 5 points in a row or 8 out of the last 10 points). In a refinement, the system quantifies real-time or near real-time “momentum” by creating a real-time or near real-time “momentum” score, indicator or other quantifiable numeric or non-numeric metric or indices (e.g., based upon how many points a player has won or lost in a row or based upon the number of consecutive or non-consecutive occurrences that exhibit one or more patterns or repetitions within any given event or scenario, from which a score, indicator, or other indices is derived). The system can be configured to modify or enhance the “momentum” score or other indices in real-time or near real-time as new data is gathered by the system. In another refinement, the “momentum” score or other indices may include at least a portion of animal data. In another refinement, the “momentum” score or other indices may utilize a weighted and/or rolling window method.

It should be appreciated that such examples of contextual data, including contextual data in the context of a sports competition/event, are merely exemplary and not exhaustive, and similar types of information can be collected for all sports and events. In the context of non-sporting events, similar types of contextual data and methodologies can be utilized. In another refinement, contextual data in the context of non-sports related events can also include outcome-related information that may or may not provide context to other data.

Still referring to FIG. 1, computing subsystem 22 can be operable to manage the one or more source sensors, as well as one or more data streams from the one or more source sensors, by at least one characteristic from the group consisting of: associated organization/entity, sensor type, sensor brand, sensor model sensor operating parameter, sensor setting/configuration, firmware information, sensor positioning, data type, data quality, data volume, associated event, timestamp, location, activity, classification, the targeted individual, groupings of targeted individuals, sensor signal, and/or data reading. In a refinement, the management and/or administration of a sensor can include functionality such as scanning for, identifying, and pairing, one or more sensors with the system, assigning one or more sensors (if required) to one or more individuals to enable association between the gathered animal data and the one or more individuals via the system, assigning the one or more sensors and/or individuals to an organization or event, associating contextual data with targeted individual, animal data, event, or a combination thereof, verifying the one or more source sensors are positioned correctly on the subject and streaming desired data once applied on subject, and the like. It can also include functionality to support the real-time or near real-time streaming of the one or more sensors to the system, including an auto-reconnect function when the one or more sensors disconnect or when a lapse in streaming occurs. In addition, the system may provide one or more alerts based on sensor disconnection, sensor failure (including battery failure), sensor degradation (e.g., producing a quality of data that does not meet a minimum established standard or threshold), checks and balances related to data quality, accuracy, repeatability, and reliability, and the like. The system can be configured to enable an administrator to establish one or more minimum thresholds related to data quality, accuracy, repeatability, reliability, speed, or a combination thereof in order to meet the one or more requirements related one or more use cases (e.g., sports betting, healthcare, fitness, insurance). In another refinement, the computing system can be operable to provide one or more alerts to one or more administrators of the system or third party computing devices based upon one or more issues with the sensor. As described herein, the one or more sensor issues may include positioning (e.g., the sensor is not in the correct position), connectivity to the skin or other apparatus (e.g., the sensor may not have a good adhesion to the skin, providing one or more faulty readings), intermediary defects (e.g., in the case of ECG sensors, the amount of electrode gel can be too much or too little to provide accurate readings), sensor defects (e.g., the sensor may be experiencing sensor degradation based upon the number of times the sensor has been used), sensor connectivity (e.g., disconnect issues), and the like. In another refinement, the computing system may run one or more simulations utilizing one or more Artificial Intelligence techniques to generate artificial data (e.g., artificial animal data) to simulate the individual's body in the given context and predict what the one or more sensor readings should look like for that particular targeted individual in that particular context (e.g., event, environment, etc.). The computing system can be configured to identify one or more issues (e.g., with the one or more sensors) based upon a comparison of the one or more readings derived from the one or more simulations and the one or more readings derived from the one or more sensors (e.g., the system identifies that the readings should look like x when they look like y and therefore the system predicts what the potential issues can be or provides one or more possibilities as to what the potential issues are).

In another refinement, the computing subsystem is operable to gather information from the one or more source sensors by communicating directly with the one or more source sensors, its associated cloud, or a native application associated with the one or more source sensors, or by communicating indirectly via one or more other computing devices in communication with the computing subsystem. In another refinement, the computing subsystem is operable to send one or more commands either directly or indirectly (e.g., via one or more other computing devices in communication with the computing subsystem) to the one or more sensors to change one or more sensor settings. For example, a command can initiate a source sensor to be turned on or off, to initiate a battery savings mode for energy saving, to initiate start or stop streaming, to increase or decrease the amount of data throughput to accommodate the bandwidth available for streaming, to change one or more processing techniques on the sensor, to change the type of data being sent by the sensor to the computing subsystem or another computing device, and the like. In a variation, a command may switch the mode of communication from sensor to system (e.g., change the wireless communication protocol from one protocol to another protocol, or enable the usage of multiple protocols together—e.g., BLE and LoRA). As another example, such commands can increase or decrease the data collection frequency and/or sensor sensitivity gain of the at least one source sensor. In another refinement, computing subsystem 22 is operable to communicate with a plurality of source sensors on a targeted individual or one or more source sensors on multiple targeted individuals simultaneously. In another refinement, computing subsystem 22 synchronizes communication and the one or more data signals or readings from multiple sensors that are in communication with the computing subsystem (e.g., which may include a combination of animal data sensors and non-animal data sensors). This includes the one or more commands sent from the sensor to the system, which may include examples such as a pre-streaming digital handshake between the sensor and the system to ensure the reliability of both parties, as well as establish one or more encryption protocols (e.g., exchange one or more messages to acknowledge each other, verify each other, establish the encryption techniques they will use, agree on session keys, or the like). It also includes addressing synchronization challenges with the one or more data signals or readings. For example, there may be a mismatch in the timings (e.g., timestamps, sampling rates) utilized by each sensor. A sensor's output received by the computing subsystem can be different (for example, by milliseconds) than another sensor even if received by the computing subsystem at the same time. Therefore, the computing subsystem can be configured to synchronize the two or more data streams to ensure that all gathered data streams are aligned. It can also include synchronizing data formats (e.g., ensuring the data is cleaned and normalized to enable further analysis), data types (e.g., ensuring data gathered in both a raw and processed format are transformed by the system to enable further analysis), and the like.

In some variations, computing subsystem 22 executes a program for generating dynamic real-time predictions using heart rate variability. When implemented, the program can be defined by an integration layer, a transmission layer, and a data management layer. With respect to the integration layer, a user or administrator of the one or more sensors enables the system to gather information from the one or more sensors in one of two ways: (1) the system communicates directly with a sensor, thereby bypassing any native system that is associated with the sensor; or (2) the system communicates with the cloud server or native system associated with the sensor, or other system that is storing the sensor data, via an API or other mechanism to collect the data into the system's database. Direct sensor communication can be achieved by either creation or modification of one or more lines of code to communicate with the sensor or the sensor manufacturer writes one or more lines of code to function with the system. The system may create a standard for communication to the system that one or more sensor manufacturers may follow. In some variations, communication between the system and the sensor can be a two-way communication where the system can receive data and send one or more commands to the sensor. For example, the system may send one or more commands to the one or more sensors to change one or more functionalities of a sensor (e.g., change the gain, power mode, or sampling rate, start/stop streaming, update the firmware) or operate in defined ways (e.g., a user may define the data collection period and communicate such operating parameters to the sensor via the program; a user selects the type of data it wants the sensor to send and the type of data it wants the sensor to store locally and communicates the one or more commands via the program). In some cases, a sensor may have multiple sensors within a device (e.g., accelerometer, gyroscope, ECG, etc.). In these cases, the system can be configured to individually control each sensor, a subset of sensors (selectable by the system), or all sensors associated with a sensor. This includes one or more sensors being turned on or off, increasing or decreasing sampling frequency or sensitivity gain, or any other functionalities or features described herein. Advantageously, the system's ability to communicate directly with the one or more sensors also enables real-time or near real-time collection of the sensor data from the sensor to the system. The system may have the ability to control any number of sensors, any number of functionalities, and stream any number of sensors on any number of targeted individuals through the single program.

With respect to the transmission layer, a byproduct of the system's direct communication with the sensor is that the system can be configured to utilize one or more techniques to elongate the transmission signal of the sensor for real-time or near real-time communication, thereby amplifying the receiving connection, increasing the communication distance between sensor and system, and extending the range limitation of the one or more sensors via the one or more transmission protocols. This can be achieved by utilizing a transmission subsystem described herein that enables the system to communicate with, and utilize, any low power or standard transmission hardware found within the sensor itself (e.g., Bluetooth, BLE, Zigbee, WIFI, cellular-based communication, Ant+, LoRa/LPWAN, and the like). Another byproduct of the system's direct communication with the sensor is that a single transmission subsystem can synchronize the communication of real-time or near real-time streaming for multiple sensors that are communicating with the system directly, and act upon the data itself, either sending it somewhere or storing it for later use. This can occur for a single individual or a plurality of individuals. The transmission subsystem can be configured any number of ways, take on various form factors, be located in any number of locations, use one or more transmission/communication protocols or networks (e.g., BLE, LoRa, ZigBee, WIFI, cellular networks, and the like), be utilized in a variety of environments, and have functionality in addition to simply transmitting data from the sensor to the system (e.g., summarizing, synthesizing or analyzing the data based on use case requirements). Advantageously, the system's direct communication with the one or more sensors via the transmission subsystem also enables real-time or near real-time streaming, particularly in hostile environments where potential interference or radio frequencies from other communications may be an issue. In a refinement, the system and sensor can achieve optimal data transmission performance by combining the usage of two or more transmission protocols (e.g., BLE and LoRa) in order communicate with the system. For example, the one or more sensors, the computing subsystem, and the transmission subsystem can be configured to utilize BLE in order to send large data files over shorter distances and utilize LoRa to send smaller data packets over longer distances. The usage of both transmission protocols by one or more sensors enables optimal data distribution to the computing subsystem in both short and long-distance scenarios. In another refinement, the system can be configured to transmit one or more commands to the one or more sensors in order to create a hybrid connectivity by enabling the usage of two or more transmission protocols (e.g., BLE and LoRa). In another refinement, the system can be configured to select one or more transmission protocols to communicate bi-directionally with the sensor (e.g., receive data from the sensor and send commands to the sensor). In another refinement, the system automatically selects the one or more transmission protocols being utilized by evaluating at least one of: data volume, data type, data requirements (e.g., by the receiving computing device), distance from sensor to the computing device, bandwidth (e.g., constraints), interference levels (e.g., RF noise levels), or a combination thereof.

With respect to the data management layer, the data management layer manages all gathered data by the system (including all animal data and its one or more derivatives), properties associated with gathered data, its associations (e.g., who/what the data is associated with), and data-related functions (e.g., normalization, synchronization, distribution, and the like, of data). Data from the one or more sensors enters the system is in one of the following structures: raw (no manipulation of the data) or processed (manipulated). The system may house one or more algorithms or other logic that deploy data noise filtering/cleaning techniques, data recovery techniques, and/or extraction or prediction techniques to extract the relevant “good” sensor data from all the sensor data (both “good” and “bad”) collected, or create artificial “good” values in the event at least a portion of the sensor data is “bad” or missing (e.g., due to a loss of connectivity or other transmission issues, battery issues, or the like). The system can be programmed to communicate with one or more sensors simultaneously that are collecting data from either a single subject or a plurality of subjects, as well as have the ability to de-duplicate them in order to transmit enough information for receiving parties to re-structure where the data is coming from and who is wearing what sensor. For clarification purposes, this means providing the system receiving the data with metadata to identify characteristics of the data—for example, a given data set belongs to timestamp A, sensor B, and subject C. In addition, the system can be configured to associate one or more sensors to one or more users. Once received by the computing subsystem, the sensor data (i.e., animal data, non-animal data, or a combination thereof will be sent to either the system cloud (e.g., cloud server) or stay local on the system's server depending on the usage of the data (e.g., request made by a third party computing device). The sensor data that enters the system is synchronized and tagged by the system with information (e.g., metadata) related to the user (e.g., characteristics/attributes), the context in which the animal data was collected (e.g., contextual data), characteristics of the system (e.g., type of computing device utilized), or characteristics of the one or more sensors including timestamps, sensor type, and sensor settings. This includes the system being operable to synchronize the sensor data with one or more timestamps and other data sources (e.g., timestamps related to the official time game clock in a basketball game, timestamps related to points scored, etc.). For example, the sensor data can be assigned to a specific user. The sensor data may also be assigned to a specific event that the user is participating in (e.g., a person playing basketball in Game X of League Y in Season Z), or a general class of activities that an acquirer of data would be interested in obtaining (e.g., group cycling data; data from a specific professional tennis match). The system, which can be schema-less and designed to ingest any type of data, will categorize the gathered sensor data by one or more characteristics including data type (e.g., ECG, EMG) and data structure. The system may take one or more further transformative actions upon the sensor data once it enters the system, the one or more actions including at least one of: normalize, timestamp, aggregate, clean, store, manipulate, denoise, process, tag, enhance, organize, categorize, analyze, anonymize, synthesize, replicate, summarize, productize, synchronize, and/or distribute the data. This will ensure consistency across disparate data sets. These processes may occur in real-time, near real-time, or on a non-real time basis depending on the use case and requirements of the user. Given the potentially large influx of data streaming or provided from the one or more sensors, which may be significant in volume, the system may also utilize a data management process that may include a hybrid approach of unstructured data and structured data schemas and formats. Additionally, the synchronization of all incoming data may use specific schema suitable for real-time or near real-time data transfer, reducing latency, providing error checking and a layer of security with an ability to encrypt parts of a data packet or the entire data packet. The system can be configured to communicate directly with other systems to monitor, receive, and record all requests for sensor data, and provide organizations that seek access to the sensor data with an ability to make one or more specific requests for data that is required for their use case. For example, one request may be for 10 minutes of real-time heart rate readings for a specific individual at a rate of 1× per second. The system can also be configured to associate those requests with one or more users or one or more groups/classes of users.

In a variation, computing subsystem 22 synchronizes, time-stamps, and tags the animal data with information (e.g., one or more characteristics) related to the one or more targeted individuals from which the animal data is collected (e.g., name, age, weight, height, activity, other contextual data) and the one or more source sensors, which can include one or more characteristics of the one or more source sensors (e.g., sensor type, one or more sensor settings/configurations, sensor brand, sensor model, sensor firmware, and the like). In a refinement, the animal data includes metadata that identifies one or more characteristics of the animal data and/or the one or more source sensors, or provides context to the collected animal data and/or the one or more source sensors. Upon data being gathered by the system, computing subsystem 22 can take one or more further actions upon the animal data, contextual data, or a combination thereof to transform the collected data. Examples of such actions include, but are not limited to, steps that normalize, timestamp, aggregate, clean, store, manipulate, denoise, process, tag, enhance, organize, categorize, visualize, analyze, anonymize, synthesize, summarize, replicate, productize, and synchronize the data.

It should be appreciated that the animal data and/or various elements of the animal data can be anonymized or de-identified (e.g., pseudonymized) by the system. De-identification involves the removal or alteration of personal identifying information in order to protect personal privacy. In the context of the present invention, a reference to one of the terms (i.e., anonymized or de-identified) should include reference to both terms and similar terms (e.g., semi-anonymized, partially-anonymized) where applicable, and a reference to one of the terms should not be interpreted as limiting but rather as encompassing all possible meanings of the terms where applicable.

Still referring to FIG. 1, computing subsystem 22 can include cloud server 40. In addition to communicating with computing subsystem 22, cloud server 40 can be operable to communicate either directly or indirectly with one or more computing devices 25, one or more computing devices 26, one or more computing devices 42, one or more sensors 18, or a combination thereof. It should be appreciated that both computing subsystem 22 and cloud server 40 can include a single computer server or a plurality of interacting computer servers. In this regard, computing subsystem 22 and cloud server 40 can communicate with one or more other systems (including each other) to monitor, receive, and record all requests for animal data to be analyzed and/or distributed (e.g., acquired) based on the one or more use cases or requirements. Moreover, computing subsystem 22 and cloud server 40 can be operable to communicate with one or more other systems—including each other—to monitor, receive, and record all requests or distributions related to animal data. Cloud server 40 can be one or more servers that are accessible via the internet or other network. Cloud server 40 can be a public cloud, a hybrid cloud, a private cloud utilized in conjunction with computing subsystem 22, a localized or networked server/storage, localized storage device (e.g., n terabyte external hard drive or media storage card), distributed network of computing devices, or the like. In a refinement, cloud server 40 is comprised of a plurality of cloud servers 40. In another refinement, cloud server 40 is associated with computing subsystem 22 and operating as part of the same system or within the same network as computing subsystem 22. In another refinement, cloud server 40 is configured to take on one or more functionalities or actions of computing subsystem 22 (e.g., in conjunction with computing subsystem 22 or separately).

In another refinement, cloud server 40 mediates the sending of animal data 14, contextual data 13, one or more primary insights 72, one or more reference insights 76, one or more predictive indicators 74, reference animal data 114, reference contextual data 113, one or more historical primary insights 172, one or more historical reference insights 176, one or more reference predictive indicators 174, or a combination thereof, to computing subsystem 22, one or more computing devices 25, one or more computing devices 26, one or more computing devices 42, or a combination thereof (i.e., it gathers the data and provides the data (e.g., makes available, sends) to one or more computing devices).

In some variations, prediction system 10 may include computing device 26. Computing device 26 can be any computing device, including a smartphone, wrist mountable unit (e.g., smart watch), a head-mountable unit (e.g., smart glasses, virtual reality or augmented reality headset), smart glasses, a desktop computer, a laptop computer, server, or any other type of computing device. In some variations, computing device 26 is local to the one or more targeted individuals or groups of targeted individuals, although not a requirement for the present invention. Computing device 26 may include an operating system that coordinates interactions between one or more types of hardware and software and operate one or more programs to gather animal data, non-animal data, or a combination thereof from one or more sensors 18 or other one or more other sources (e.g., another one or more computing devices; one or more programs that enable animal data to be manually inputted; and the like). In a refinement, computing device 26 mediates the sending of animal data 14 (or contextual data 13 or a combination thereof) to computing subsystem 22, cloud server 40, computing device 25, or one or more computing devices 42, i.e., it collects the animal data from one or more sensors 18, as well as from any programs operating on computing device 26 that gathers animal data, and transmits it to computing subsystem 22, cloud server 40, computing device 25, one or more computing devices 42, or a combination thereof. In another refinement, one or more computing devices 26 operate in conjunction with computing subsystem 22, cloud server 40, computing device 25, one or more computing devices 42, or a combination thereof. For example, one or more computing devices 26 may operate separately from computing subsystem 22 (e.g., different hardware and software) but may provide one or more animal data outputs (or non-animal data outputs) to computing subsystem 22. In another refinement, one or more computing devices 26 operate as part of computing subsystem 22. In another refinement, computing device 25 is configured to take on one or more functionalities or actions of computing subsystem 22 (e.g., in conjunction with computing subsystem 22 or separately). In another refinement, computing device 26 operates as computing subsystem 22.

Characteristically, computing device 26 may include one or more features of transmission subsystem 24. In a refinement, transmission subsystem 24 can include computing device 26 which mediates the sending of animal data 14 to computing subsystem 22, i.e., it collects the data and transmits it to computing subsystem 22. In another refinement, one or more sensors 18 can be housed within, attached to, affixed to, or integrated with, computing device 26 (e.g., as in the case of a computing device such as a smart watch, smart glasses, smart clothing, augmented reality headset, any other bodily-mountable unit, and the like which includes one or more sensors 18 that collect animal data). In another refinement, one or more sensors 18 can be operable to communicate either directly or indirectly with one or more computing devices 26. In another refinement, computing device 26 is comprised of a plurality of computing devices 26.

Still referring to FIG. 1, computing subsystem 22 accesses reference animal data 114, reference contextual data 113, one or more historical primary insights 172, one or more historical reference insights 176, one or more reference predictive indicators 174, or a combination thereof, via one or more computing devices 25. Computing device 25 can be any computing device capable of taking one or more actions related to the reference animal data (e.g., gathering reference animal data, actioning upon/transforming reference animal data, and/or providing reference animal data to another one or more computing devices). The one or more transformative actions can include, but are not limited to, steps that normalize, timestamp, aggregate, clean, store, manipulate, denoise, process, enhance, organize, categorize, tag, visualize, analyze, anonymize, synthesize, summarize, replicate, productize, and synchronize the reference animal data. In a refinement, one or more computing devices 25 provide (e.g., send, make available) reference animal data 114, reference contextual data 113, one or more historical primary insights 172, one or more historical reference insights 176, one or more reference predictive indicators 174, or a combination thereof to computing subsystem 22, cloud server 40, one or more computing devices 42, or a combination thereof.

In a refinement, one or more computing devices 25 can operate as a separate one or more computing devices with different functionalities as one or more computing subsystems 22 or clouds 40, or it can operate as separate computing device with one or more shared functionalities as one or more computing subsystems 22 or clouds 40 (e.g., computing device 25, computing subsystem 22, cloud server 40, or a combination thereof, can be configured to take one or more coordinated actions on the same animal data or reference animal data). In another refinement, computing device 25 is configured to operate, at least in part, as computing subsystem 22 or cloud 40. In another refinement, computing subsystem 22 and/or cloud 40 is configured to operate, at least in part, as computing device 25. In another refinement, one or more of the actions taken by computing device 25 are operable to be taken by computing subsystem 22 or cloud server 40. In another refinement, computing device 25 is part of computing subsystem 22 (i.e., computing device 25 is not a separate computing device from computing subsystem 22—computing device 25 can be integrated with or attached to computing subsystem 22 or they are the same computing device). In another refinement, computing subsystem 22 or cloud 40 is configured to take on one or more functions or features of (e.g., take one or more actions on behalf of) computing device 25. In another refinement, computing device 25, computing subsystem 22, cloud 40, or a combination thereof, operate using the same one or more computing devices. In another refinement, computing device 25, computing subsystem 22, cloud 40, or a combination thereof, operate using different computing devices. In another refinement, computing device 25 can operate the same one or more programs as computing subsystem 22 or different one or more programs to gather (e.g., receive, collect), action upon (e.g., transform), and/or provide (e.g., send, make available) animal data to computing subsystem 22, cloud server 40, computing device 26, or a combination thereof. In another refinement, computing device 25 is comprised of a plurality of computing devices 25. In another refinement, computing device 25, computing subsystem 22, cloud server 40, computing device 26, or a combination thereof, coordinate one or more actions to gather, action upon (e.g., transform) and/or provide animal data (e.g., including reference animal data, its one or more derivatives, and the like).

Still referring to FIG. 1, one or more individuals 191 are the one or more subjects from which reference animal data 114 corresponds with. One or more individuals 191 can include one or more targeted individuals 16ⁱ, as well as other individuals with associated animal data in the reference animal database. Reference animal data 114 can be reference animal data that is directly or indirectly related to one or more individuals 191. Reference animal data 114 from the one or more individuals 19ⁱ along with reference contextual data 113 are included in the reference animal database from which the one or more reference insights will be created, modified, or enhanced.

Reference animal data 114 and reference contextual data 113 can be gathered (e.g., inputted, imported, collected) by one or more computing devices 25 via one or more sensors 18, one or more other computing devices (e.g., one or more computing subsystems 22, clouds 40, computing devices 26, or third-party computing devices 42), or a combination thereof. In this regard, the one or more computing devices 25 can be the one or more computing devices from which the reference animal data 114 and reference contextual data 113 is gathered, stored, actioned upon/transformed, and/or made available (e.g., distributed). Reference animal data 114 and reference contextual data 113 can be gathered, stored, transformed, and/or made available by a single computing device 25 or across multiple computing devices 25. In some variations, the one or more computing devices that gather reference animal data 114 and reference contextual data 113 are different from the one or more computing devices that store the reference animal data and/or the reference contextual data or make available the reference animal data or reference contextual data (e.g., to create, modify, or enhance the at least one reference insight). In other variations, the one or more computing devices that gather the reference animal data 114 and/or the reference contextual data 113 are the same as the one or more computing devices that store and make available the reference animal data and/or the reference contextual data.

Reference animal data 114 and reference contextual data 113 can be accessed by a single computing device or multiple computing devices. In a refinement, one or more computing subsystems 22 or clouds 40 can access reference animal data 114 and reference contextual data 113 in order to create, modify, or enhance one or more reference insights. In another refinement, the reference animal data and/or reference contextual data is gathered from one or more other external systems (e.g., one or more computing devices 42). In a further refinement, the reference animal data gathered from one or more computing devices or other external systems has attached metadata that enables the reference animal data to be associated with one or more subjects, events, sensors, data type, and the like.

In another refinement, the reference animal data 114 is associated with reference contextual data 113 in order to provide context for reference animal data 114. For example, reference contextual data 113 can be included as part of the metadata for reference animal data 114 or separately synced with reference animal data 114 to enable analysis. In another variation, depending on the data, at least a portion of reference animal data 114 can be classified as reference contextual data 113 and vice versa. In another variation, depending on the data, at least a portion of animal data 14 can also be classified as contextual data 13 and vice versa. In another refinement, the term “reference data” can include reference animal data, reference contextual data, or a combination thereof.

Characteristically, animal data 14 from one or more individuals 16 and one or more sensors 18, as well as contextual data 13, can be collected by one or more computing subsystems 22, computing devices 26, or clouds 40 and provided to one or more computing devices 25 as reference animal data 114 and reference contextual data 113 respectively once the animal data is associated with the one or more individuals 16, one or more events, or a combination thereof. In a refinement, association occurs via the system creating, modifying, or enhancing one or more digital records which are included as part of the reference animal database. In a refinement, one or more reference animal databases can be created for each individual, a subset of individuals, or all individuals. The system can be configured to gather the animal data and corresponding contextual data (e.g., the one or more variables that affect the animal data readings, sensor information, and the like) along with the one or more patterns, rhythms, trends, features, measurements, outliers, abnormalities, anomalies, characteristics/attributes, and the like that are unique to the individual, including the one or more changes, variations, or similarities in the animal data based upon the one or more variables, and create one or more digital records that enables the system to associate the animal data, the metadata, and the one or more changes or variations with the individual for that given context. The one or more digital records are included as part of the reference animal data 114 database and can include each individual 19ⁱ's animal data and other information (e.g., attributes, other metadata), representing that individual's reference animal data 114. The system may store the reference animal data, reference contextual data, and associated information as part of the one or more digital records in the reference animal database associated with that individual, event, sensor, data type, or the like and access the one or more digital records to create, modify, enhance, or access one or more reference insights. In another refinement, reference animal data and the corresponding reference contextual data is organized and searchable via one or more digital records that are associated with (1) each subject, event, sensor, data type (e.g., animal data type, contextual data type), and the like; (2) a plurality of subjects, events, sensors, data type, and the like; (3) one or more characteristics related to each subject, event, sensor, data type, and the like; (4) one or more characteristics related a plurality of subjects, events, sensors, data type, and the like; or (5) a combination thereof. In a variation, one or more tags can be created based upon one or more characteristics related to the reference animal data and/or reference contextual data (e.g., including contextual information and other metadata), the one or more targeted subjects (e.g., including their one or more medical conditions or biological responses), the one or more sensors, the one or more events, or a combination thereof. In a refinement, one or more tags can also be created based upon their one or more characteristics. Tags (e.g., including classifications or groups that a targeted subject can be assigned to such as soccer team, individuals with a specific type of disease or blood type, and the like, or classifications or groups that medical conditions or biological responses may be assigned to) can be identifiers for data, can support the indexing and search process for one or more computing devices or data acquirers (e.g., tags can simplify the search process as one or more searchable tags), and can be based on data collection processes, practices, quality, or associations, as well as targeted individual characteristics. In another refinement, the same reference animal data 114 or reference contextual data 113 are included as part of two or more digital records. In another refinement, the one or more digital records can be modified or enhanced as new data is gathered the system.

Still referring to FIG. 1, in many variations at least one reference insight 76 is created, modified, or enhanced from reference animal data 114 and contextual data 113 by one or more computing devices 25. A reference insight can be quantified by one or more numbers (e.g., including a plurality of one or more numbers), and can be represented as a probability or similar odds-based indicator. A reference insight may also be quantified, communicated, or characterized by one or other metrics or indices of animal data-based performance that are predetermined (e.g., codes, graphs, charts, plots, colors or other visual representations, readings, numerical representations, descriptions, text, physical responses such as a vibration, auditory responses, visual responses, kinesthetic responses, verbal descriptions, and the like). A reference insight may also include one or more visual representations related to a condition or status of the of one or more targeted subjects (e.g., an avatar or virtual depiction of a targeted subject visualizing future weight loss goals on the avatar or depiction of the targeted subject). In a refinement, a reference insight is derived from two or more types of animal data. In another refinement, a reference insight is derived related to a targeted subject or group of targeted subjects using at least a portion of animal data not derived from the targeted subject or group of targeted subjects. In another refinement, a reference insight is comprised of a plurality of reference insights. In another refinement, a reference insight is assigned to a single targeted individual. In another refinement, a reference insight is assigned to multiple targeted individuals. In another refinement, a reference insight is assigned to one or more groups of targeted individuals. In another refinement, a reference insight is created, modified, or enhanced using one or more Artificial Intelligence techniques. In another refinement, a created, modified, or enhanced reference insight is used as training data for one or more Artificial Intelligence-based techniques to create, modify, or enhance of one or more predictive indicators. In another refinement, a reference insight includes the event outcome and at least a portion of the animal data, contextual data, one or more derivatives, or a combination thereof, associated with the event outcome (e.g., as selected by the system, defined by the user, or the like). In another refinement, a reference insight is created, modified, or enhanced via one or more simulations. In another refinement, a reference insight includes at least a portion of simulated data. In a variation, a reference insight refers to the association of one or more Variability Indicators and thresholds to one or more outcomes when applied to/observed in the reference data/observations, wherein the one or more Variability Indicators in the reference data are calculated using the one or more methods described herein.

In a refinement, the at least one reference insight 76 is created, modified, or enhanced from reference animal data 114 and contextual data 113 on a single computing device. In another refinement, the at least one reference insight 76 is created, modified, or enhanced from reference animal data 114 and contextual data 113 on two or more computing devices. In another refinement, the at least one reference insight 76 is created, modified, or enhanced via one or more sensors (e.g., including one or more unmanned aerial vehicles or other computing apparatus with one or more sensors integrated or attached and computing capabilities to create, modify, or enhance the at least one reference insight). In another refinement, the at least one reference insight 76 is included as part of reference animal data 114. In a variation, the at least one reference insight 76 included as part of reference animal data 114 can be modified, enhanced, or removed by the system. In another refinement, the one or more computing devices take one or more of the following actions on the collected reference animal data 114 and/or the reference contextual data 113 to transform the reference animal data and/or the contextual data 113 into at least one reference insight 76: normalize, timestamp, aggregate, clean, tag, store, manipulate, denoise, tag, process, enhance, organize, categorize, analyze, visualize, simulate, anonymize, synthesize, summarize, replicate, productize, synchronize, or distribute the data.

In a variation, one or more computing devices 25 are operable (e.g., configured) to create, modify, or enhance the at least one reference insight 76 from one or more calculations, computations, combinations, measurements, derivations, extractions, extrapolations, simulations, creations, modifications, enhancements, estimations, evaluations, inferences, establishments, determinations, conversions, deductions, or observations. In a refinement, the at least one reference insight 76 is created, modified, or enhanced via computing subsystem 22, cloud 40, computing device 25, or a combination thereof.

In a refinement, the system can be configured to gather the reference animal data 113 and associated reference contextual data 114 (e.g., the one or more variables that can affect the animal data) to create, modify, or enhance the at least one reference insight from one or more patterns, rhythms, trends, features, measurements, outliers, abnormalities, anomalies, characteristics/attributes, and the like (“Identifiers”) derived, at least in part, from the sensor-based animal data that are associated with a specific outcome for any given event (e.g., including its one or more sub-events) and unique to the individual or unique to a subset of individuals, including the one or more changes or variations in the animal data based upon the one or more variables that are associated with the specific outcome for an event (e.g., patterns/trends that occurred resulting in the outcome, such as a point win/loss in a sports competition). In another refinement, the at least one reference insight includes the one or more Identifiers. In another refinement, the at least one reference insight is comprised of, at least in part, the one or more Identifiers and the event outcome. In another refinement, the at least one reference insight can be created, modified, enhanced, or accessed dynamically (e.g., on the fly) at the direction of the system based upon (1) the type of animal data being gathered by the system, (2) the associated contextual data (e.g., the one or more types of sensors being utilized, including their one or more associated operating parameters; the event; the point in time within the event; the environment; momentum; and the like), or (3) the reference animal data (e.g., including insights derived from it).

Still referring to FIG. 1, in the variations in which at least one reference insight 76 is created, modified, or enhanced, computing subsystem 22 implements one or more methods described herein to create, modify, or enhance at least primary insight 72. At least one primary insight 72 can be created, modified, or enhanced from reference animal data 14 and contextual data 13 by computing subsystem 22 via one or more calculations, computations, combinations, measurements, derivations, extractions, extrapolations, simulations, creations, modifications, enhancements, estimations, evaluations, inferences, establishments, determinations, conversions, deductions, or observations. The at least one primary insight 72 can be created, modified, or enhanced in real-time or near real-time. In a refinement, the at least one primary insight 72 is created, modified, or enhanced via computing subsystem 22, cloud 40, computing device 25, computing device 26, sensor 18, or a combination thereof.

A primary insight can be quantified by one or more numbers (e.g., including a plurality of one or more numbers), and can be represented as a probability or similar odds-based indicator. A primary insight may also be quantified, communicated, or characterized by one or other metrics or indices of animal data-based performance that are predetermined (e.g., codes, graphs, charts, plots, colors or other visual representations, readings, numerical representations, descriptions, text, physical responses such as a vibration, auditory responses, visual responses, kinesthetic responses, verbal descriptions, and the like). A primary insight may also include one or more visual representations related to a condition or status of the of one or more targeted subjects (e.g., an avatar or virtual depiction of a targeted subject visualizing future weight loss goals on the avatar or depiction of the targeted subject). In a refinement, a primary insight is derived from two or more types of animal data. In another refinement, a primary insight is derived related to a targeted subject or group of targeted subjects using at least a portion of animal data not derived from the targeted subject or group of targeted subjects. In another refinement, a primary insight is comprised of a plurality of primary insights. In another refinement, a primary insight is assigned to a single targeted individual. In another refinement, a primary insight is assigned to multiple targeted individuals. In another refinement, a primary insight is assigned to one or more groups of targeted individuals. In another refinement, a created, modified, or enhanced primary insight is used as training data for one or more Artificial Intelligence-based techniques to create, modify, or enhance of one or more predictive indicators. In yet another refinement, a primary insight includes the event outcome and at least a portion of the animal data, contextual data, one or more derivatives, or a combination thereof, associated with the event outcome (e.g., as selected by the system, defined by the user, or the like). In a further refinement, the primary insight may include a plurality of event outcomes.

In a refinement, the at least one primary insight 72 is created, modified, or enhanced from animal data 14 and contextual data 13 on a single computing device. In another refinement, the at least one primary insight 72 is created, modified, or enhanced from animal data 14 and contextual data 13 on two or more computing devices. In another refinement, the at least one primary insight 72 is created, modified, or enhanced via one or more sensors (e.g., on a wearable or optical sensor, on one or more unmanned aerial vehicles or other computing apparatus with one or more sensors integrated or attached and computing capabilities to create, modify, or enhance the at least one primary insight). In another refinement, the one or more computing devices take one or more of the following actions on the collected animal data 14 and/or the contextual data 13 to transform the animal data and/or the contextual data 13 into at least one primary insight 72: normalize, timestamp, aggregate, clean, tag, store, manipulate, denoise, process, enhance, organize, categorize, analyze, visualize, simulate, anonymize, synthesize, summarize, replicate, productize, synchronize, or distribute the data. In a refinement, a primary insight is created, modified, or enhanced using one or more Artificial Intelligence techniques. In another refinement, a primary insight is created, modified, or enhanced via one or more simulations. In another refinement, a primary insight includes at least a portion of simulated data.

In a refinement, the system can be configured to gather the animal data 13 and its associated contextual data 14 (e.g., the one or more variables that affect the animal data readings, sensor information, and the like) and create, modify, or enhance the at least one primary insight from one or more patterns, rhythms, trends, features, measurements, outliers, abnormalities, anomalies, characteristics/attributes, and the like (“Identifiers”) derived, at least in part, from the sensor-based animal data that are associated with any given event, including one or more changes or variations in the animal data based upon the one or more variables that are associated with the event. In a refinement, the sensor-based animal data is also associated with a specific outcome for the given event. In another refinement, the one or more primary insights include the one or more Identifiers. In another refinement, the one or more primary insights are comprised of, at least in part, the one or more Identifiers and the event outcome. In another refinement, the one or more Identifiers are unique to the individual or unique to a subset of individuals, (e.g., patterns/trends that occurred during the course of one or more events). In another refinement, a primary insight can become a reference insight upon one or more other primary insights being created, modified, or enhanced. In another refinement, the one or more primary insights can be created dynamically (e.g., on the fly) at the direction of the system based upon (1) the type of animal data being gathered by the system, (2) the associated contextual data (e.g., the one or more types of sensors being utilized, including their one or more associated operating parameters; the event; the point in time within the event; the environment; momentum; and the like), or (3) the reference animal data (e.g., including insights derived from it).

In a refinement, upon being created, modified, or enhanced by the system, at least one reference insight 76 can become one or more historical reference insights 176 and made available as an input in one or more creations, modifications, or enhancements of the at least one reference insight 76 or predictive indicator 74 and/or distributed to another one or more computing devices. In another refinement, upon being created, modified, or enhanced by the system, at least one primary insight 72 can become one or more historical primary insights 172 and made available as an input in one or more creations, modifications, or enhancements of the at least one reference insight 76 or predictive indicator 74 and/or distributed to another one or more computing devices. In another refinement, upon being created, modified, or enhanced by the system, at least one predictive indicator 74 can become one or more reference predictive indicators 174 and made available as an input in one or more creations, modifications, or enhancements of the at least one reference insight 76 or predictive indicator 74 and/or distributed to another one or more computing devices (e.g., for consideration). In another refinement, one or more historical primary insights 172, historical reference insights 176, or reference predictive indicators 174 are included as part of reference animal data 114. In a refinement, previous predictive indicators that are based upon the animal data can become part of the reference animal data. For example, if the predictive indicator changes from n % to y % chance of winning a match, the n % can become part of the reference animal data. In this context, the n % would also be classified as a reference predictive indicator.

In a variation, upon creating, modifying, or enhancing at least one primary insight 72, computing subsystem 22 utilizes the at least one reference insight 76 and primary insight 72 to create, modify, or enhance predictive indicator 74. The at least one predictive indicator 74 can be created, modified, or enhanced in real-time or near real-time. In a refinement, the at least one reference insight 76, primary insight 72, or predictive indicator 74 is created, modified, or enhanced via computing subsystem 22, cloud 40, computing device 25, computing device 26, or a combination thereof. In another refinement, the at least one reference insight 76, primary insight 72, or predictive indicator 74 are considered to be animal data.

In a refinement, the system can compare the animal data and the derived one or more Identifiers for a targeted individual in a specific context for an event (i.e., the primary insight) with reference animal data featuring one or more Identifiers created, modified, enhanced, or gathered from previously-collected animal data (e.g., which may have been previously collected in the prior seconds, minutes, hours, days, weeks, years, and the like) in the same or similar context, at least in part, and associated with one or more previous event outcomes (i.e., the reference insight), to create, modify, or enhance one or more predictive indicators. For example, the system may gather a variety of animal data from one or more sensors (e.g., a plurality of animal data simultaneously) either directly or indirectly (e.g., via another computing device) and identify one or more patterns in the collected animal data (e.g., in a subject's ECG, heart rate variability data, and breathing rate data) in light of the one or more variables (e.g., context in which the data was collected, the one or more characteristics/attributes of the individual, and the like, the information of which is included in the metadata). This gathered animal data and metadata becomes reference animal data and reference contextual data. The system is trained using one or more AI techniques to learn the one or more patterns in a variety of contexts—which can be different based on the one or more variables—along with what the associated outcome was based upon the one or more patterns to create one or more reference insights that are included as part of the one or more digital records associated with the individual, the animal data, the one or more sensors, the contextual data (e.g., the event), or a combination thereof. The collection of the one or more Identifiers in each of the animal data types, or each of the one or more Identifiers, and the associated outcome with each Identifier or a subset of one or more Identifiers comprises the one or more reference insights created for the individual. The system then collects animal data from one or more source sensors in a real-time or near real-time manner with contextual data. The system can be configured to enable a user (or an AI) to create or modify one or more search or identification parameters—which are tunable based upon the gathered animal data, contextual data, or a combination thereof—that enable the identification or creation of one or more Identifiers derived from (e.g., in, associated with, or the like) the animal data. The one or more Identifiers derived from the animal data comprises the one or more primary insights. The system then takes the one or more primary insights and evaluates them in light of the one or more reference insights. The evaluation enables the system to create, modify, or enhance one or more predictive indicators.

In a refinement, the at least one reference insight, the at least one primary insight, the at least one predictive indicator, or a combination thereof, are used as training data for one or more Artificial Intelligence techniques to create, modify, or enhance one or more new predictive indicators (e.g., create a new predictive indicator, enhance an existing predictive indicator, making it a new predictive indicator). For example, once a prediction for an event has occurred, the system can look at the real-world outcome of the event to determine the efficacy and accuracy of the prediction. The system can then utilize the at least one reference insight, at least one primary insight, the at least one predictive indicator, or a combination thereof, as training data—the system taking one or more actions to transform (e.g., categorize, tag, or the like) the at least one reference insight 76 as a historical reference insight 176, the at least one primary insight 72 a historical primary insight 172, and the at least one predictive indicator 74 a reference predictive indicator 174—to create, modify, or enhance one or more new predictive indicators (e.g., a predictive indicator for the next event or future event; another predictive indicator) in order to enhance the accuracy of predictions for future events. Characteristically, this enables the system to generate real-time or near real-time predictions dynamically as upon new animal data enters the system (e.g., which enables new or enhanced reference insights, new or enhanced primary insights, new or enhanced predictive indicators, or a combination thereof).

Still referring to FIG. 1, upon creating, modifying, or enhancing or gathering the at least one predictive indicator, reference insight or primary insight, computing subsystem 22 or cloud 40 distributes the animal data, contextual data, or a combination thereof, to one or more computing devices 42. Computing device 42 is any computing device (e.g., which includes systems operating on that computing device) that can gather information (e.g., receive animal data) provided by another computing device either directly or indirectly, or provide information related to one or more subjects. The one or more computing devices 42 are typically the acquirers of the animal data (e.g., third parties), but can be an intermediary server that provides data to another one or more parties. One or more computing devices 42 (e.g., third-party computing devices 42) can include sports media systems (e.g., for displaying or communicating the collected data such as a sports broadcast system, video streaming platform, sports fan engagement system, and the like), sports wagering systems, data visualization systems, e-sports and video gaming systems, risk analytics systems, animal performance analytics systems, health and wellness monitoring systems, research systems, fitness systems, and the like. In some variations, one or more computing devices 42 can be the operator a sports wagering system (e.g., sports betting web application, native mobile application, in-venue platform such as a casino, restaurant/bar, arena/venue, and the like). In other variations, one or more computing devices 42 can act as a source for reference animal data 114 or contextual data 113 to one or more computing devices 25, or take one or more actions on reference animal data 114 and reference contextual data 113 to transform the data.

One or more computing devices 42 can be comprised of a single computing device or multiple computing devices as part of one or more systems. One or more computing devices 42 can include an operating system that coordinates interactions between one or more types of hardware and software. One or more computing subsystems 22, cloud servers 40, or a combination thereof, can communicate either directly or indirectly with one or more computing devices 42 via one or more communication links (e.g., wireless, wired, or a combination thereof). In some variations, one or more computing devices 42 is part of computing subsystem 22. In other variations, one or more computing devices 42 are operated separately from computing subsystem 22. In other variations, one or more computing devices 42 operate separately from each other. For example, one computing device 42 can be used for one function, while another computing device 42 can be used for another function. In this variation, the two computing devices are part of the same system and may or may not be in communication with, or associated with, each other. In another variation, two or more computing devices are not part of the same system (e.g., computing subsystem 22 sends data via cloud server 40 to separate computing devices 42 which operate independently of one another). In a refinement, one or more computing devices 42 can be operated by the same entity operating computing subsystem 22 (e.g., as in the case of a sports betting operator that also provides computing subsystem 22 to collect and the data and generate the one or more predictive indicators) or by one or more different entities. In another refinement, the one or more computing devices 42 are not part of computing subsystem 22 but operated by the same entity operating computing subsystem 22 or one or more different entities. In another refinement, computing device 42 incudes a plurality of computing devices 42.

Computing device 42 can also include systems located on the one or more targeted individuals (e.g., another wearable with a display such as a smartwatch, smart glasses, or virtual reality/augmented reality headset) or other individuals interested in accessing the targeted individual's data (e.g., a sports bettor interested in accessing the animal data from one or more targeted individual athletes on their computing device such as their mobile phone). In a refinement, one or more sensors 18 can be operable to communicate either directly or indirectly with one or more computing devices 42. In another refinement, one or more computing devices 25 are operable to communicate either directly with one or more computing devices 42.

In a refinement, computing subsystem 22 mediates the sending of animal data 14, contextual data 13, one or more primary insights 72, one or more reference insights 76, one or more predictive indicators 74, reference animal data 114, reference contextual data 113, one or more historical primary insights 172, one or more historical reference insights 176, one or more reference predictive indicators 174, or a combination thereof, to cloud server 40, computing device 25, computing device 26, or computing device 42 (i.e., it collects the animal data from one or more sensors 18, as well as from any programs operating on computing subsystem 22 or in communication with computing subsystem 22 that gathers animal data and transmits it to cloud server 40, computing device 25, computing device 26, or computing device 42, or a combination thereof).

In another refinement, one or more computing devices 42 provide animal data (e.g., including its one or more derivatives such as products that incorporate animal data) to one or more data acquirers for consideration (e.g., payment, a reward, a trade for something of value which may or may not be monetary in nature). In another refinement, one or more computing devices 42 distribute at least a portion of the consideration to at least one stakeholder 30 (e.g., computing device 30). The one or more stakeholders can be a user that produced (e.g., generated) the data (e.g., the targeted subject from which the animal data is derived, such as an athlete), the owner of the data (e.g., which could be the same individual who produced the data or different), the data collection company, authorized distributor of the animal data, a sensor company (e.g. a sensor company that collected the acquired animal data), an analytics company (e.g., an analytics company that provided analytics on the acquired data), an application company, a data visualization company, a cloud server company that operates the cloud server, a company that operates one or more computing devices that stores the reference animal data (which is used to verify the association between the targeted individual and their animal data), a company that develops products based on the animal data, or any other entity (e.g., typically one that provides value to any of the aforementioned stakeholders or the data acquirer).

Still referring to FIG. 1, Computing subsystem 22, computing device 25, computing device 26, one or more computing devices 42, or a combination thereof, can include a display device that enables a user (e.g., a subject utilizing one or more sensors from which animal data is collected; an administrator operating the system on behalf of a subject utilizing one or more sensors from which animal data is collected, and the like) to take one or more actions within the display (e.g., touch-screen enabling an action; use of a scroll mouse that enables the user to navigate and make selections; voice-controlled action via a virtual assistant or other system that enables voice-controlled functionality; eye-tracking within spatial computing systems that enables an eye-controlled action; a neural control unit that enables one or more controls based upon brain waves; and the like). In a refinement, a gesture controller that enables limb (e.g., hand) or body movements to indicate an action can be utilized to take one or more actions. In a refinement, the display may act as an intermediary to communicate with another one or more computing devices to execute the one or more actions requested by the user.

Typically, a display device communicates information in visual form, and allows for two-way communication (e.g., the display enables a subject to take one or more actions via the display, such as place a bet). However, a display device may communicate information to a user, and receive information from a user, utilizing one or more other mechanisms including via an audio or aural format (e.g., verbal communication of information such as biological readings), via a physical gesture (e.g., a physical vibration which provides information related to the one or more biological readings, a physical vibration which indicates when the data collection period is complete, or a physical gesture to induce a biological-based response from the individual's body can be captured as animal data via one or more sensors), or a combination thereof. In some variations, the information communicated to or provided by a user can be animal data-based information such as the type of animal data, activity associated with the animal data or other metadata (e.g., contextual data), insights or predictive indicators, and the like. For example, the display device may not communicate the signals or readings associated with the animal data for the user to interact with but may communicate the type of animal data (e.g., the display may not provide a user's actual heart rate values but may display the term “heart rate” or “HR” or a symbol related to heart rate—such as a heart—which the user can select and define terms related to their heart rate data). In a refinement, the display device can also provide an ability for the user to communicate information with the system (e.g., ability for a user to provide one or more inputs to operate the program, provide requested information to the system, and the like). In another refinement, the display may not include any visual component in its communication or receipt of information (e.g., as in the case of a smart speaker, hearables, or similar computing device that does not include any visual screen to interact with and is operable via a virtual or audio-based assistant to receive one or more commands and take one or more actions. In this example, the smart speaker or hearables can be in communication with another computing device to visualize information via another display if required).

A display device may include a plurality of display devices that comprise the display. In addition, a display that is not included as part of computing subsystem 22, computing device 25, computing device 26, and/or one or more computing devices 42 can be in communication with computing subsystem 22, computing device 25, computing device 26, and/or one or more computing devices 42 (e.g., attached or connected to, from which communication occurs either via wired communication or wirelessly). Furthermore, the display device may take one or more forms. Examples of where one or more types of animal data can be displayed include via one or more monitors (e.g., via a desktop, laptop computer, projector), holography-based computing devices, smart phone, tablet, a smart watch or other wearable with an attached or associated display, smart speakers (e.g., including earbuds/hearables), smart contact lens, smart clothing, smart accessories (e.g., headband, wristband), or within a head-mountable unit (e.g., smart glasses or other eyewear/headwear including virtual reality/augmented reality headwear) where the animal data (e.g., insight, predictive indicator, and the like) or other animal data-related information can be visualized or communicated. In a refinement, the display can be operating as part of, or displaying receiving animal data or animal data-related information (or other information requested by the system) via one or more programs that include or are related to, but not limited to, a sports wagering system, media system, fan engagement system, health monitoring system, fitness system (e.g., a home fitness or gym application that enables users to view or access their animal data), health passport system, telehealth system, animal data monetization system (e.g., including animal data marketplaces, systems for providing loans using animal data as collateral, at least in part, or as part of an animal data-based digital currency system), insurance system, animal performance system (e.g., human performance optimization system), health analytics system, electronic medical records system, electronic health records system, risk analytics system (e.g., insurance, insurance underwriting, finance, security), pharmaceutical-based system (e.g., drug administration system), performance analytics system, health and wellness monitoring system (e.g., including systems to monitor viral infections), research system, security/integrity system (e.g., subject or sensor identification/verification/authentication for security purposes; system that identifies and/or verifies fraudulent behavior), military system, hospital system, emergency response system, financial system, banking system, relationship management system, social media system, simulation/video game system (e.g., virtual world, metaverse), media & entertainment system, and the like. In another refinement, the display may include one or more other media streams (e.g., live-stream video, digital objects). For example, computing device 42, which may operable a mobile-based sports betting application, may include an integrated display that enables both the visualization of media (e.g., video of a live sports event) and the real-time or near real-time data (e.g., animal data or its one or more derivatives—e.g., one or more bets utilizing at least a portion of animal data—and contextual data). In another example, a home fitness machine (e.g., rowing machine) may include an integrated display that enables both the visualization of media (e.g., video of a fitness instructor) along with the real-time animal data, or a computing device can be operating health monitoring program (e.g., telehealth application) which may include an integrated media module (e.g., real-time video of a doctor or medical professional with two-way video and voice communication) within the display alongside the real-time animal data being communicated (e.g., visualized) by the system, or a virtual environment may that includes a variety of digital objects may also incorporate animal data or animal data-based information in the virtual world, and the like. In another refinement, computing device 42 take one or more actions to sync one or more video and/or audio streams with the animal data and at least a portion of non-animal data (e.g., timing & scoring data, other contextual data) to and provides at least a portion of the synced media to one or more computing devices, displays, or a combination thereof. Such information can be provided via one or more cloud or other intermediary servers. In another refinement, computing device 42 creates one or more graphical displays for the animal data within the video display.

Still referring to FIG. 1, one or more Artificial Intelligence techniques can be utilized as part of the one or more methods or systems to create, modify, or enhance one or more reference insights, primary insights, or predictive indicators, as well as to execute any one of the steps required in the one or more methods to collection, transform, and distribute data (e.g., derive R-R intervals, calculate one or more heart rate variability values, calculating one or more heart rate variability baselines, and the like). For definition purposes, Artificial Intelligence includes, but is not limited to, Machine Learning, Deep Learning, Statistical Learning, and the like. The one or more Artificial Intelligence techniques can also be utilized to dynamically create, modify, or enhance one or more reference insights, primary insights, or predictive indicators based upon new data (e.g., contextual data including animal data, non-animal data, or a combination thereof) being gathered by the system. For example, a variable in the at least one reference insight (e.g., win percentage) can be modified or enhanced by Machine Learning techniques based upon new contextual data entering the system to create a new reference insight. Characteristically, the one or more creations, modifications, or enhancements can occur (e.g., dynamically) in real-time or near real-time. For example, by utilizing one or more Artificial Intelligence techniques such as Machine Learning techniques, the system can analyze both reference animal data, reference contextual data, animal data, and contextual data derived from the one or more sensors and/or computing devices to create, modify, or enhance one or more reference insights, primary insights, or predictive indicators. In a refinement, the one or more outputs of one AI technique is used as one or more inputs of another one or more techniques to create, modify, or enhance one or more predictive indicators (e.g., increase the predictive power, reduce error, and the like).

In the case of Machine Learning-based techniques, given that Machine Learning-based systems are set up to learn from collected data rather than require explicit programmed instructions, its ability to search for and recognize patterns that can be hidden within the reference animal data and the gathered sensor data from the one or more source sensors enable Machine Learning and other AI-based systems to uncover insights from collected data that allow unique biological-based information to be uncovered for each individual based upon their animal data. Thus, the one or more reference insights, primary insights, and/or predictive insights can be customized and tailored for each individual. Advantageously, because Machine Learning and Deep Learning-based systems use data to learn, it oftentimes takes an iterative approach to improve model prediction and accuracy as new data enter the system, as well as improvements to model prediction and accuracy derived from feedback provided from previous computations made by the system (which also enables production of reliable results). In such a scenario, new animal data or contextual from the one or more source sensors or computing devices, as well as new reference animal data entering the system at any given time, enables a new, deeper understanding of the user and potential outcomes based upon a broader set of data.

By utilizing one or more Artificial Intelligence techniques such as Machine Learning, Statistical Learning, or Deep Learning techniques, the system can identify one or more patterns, rhythms, trends, features, measurements, outliers, abnormalities, anomalies, characteristics/attributes, or the like in the reference animal data that make each individual data set unique or identifiable when compared the other reference animal data sets (thereby making each individual and each data set associated with each individual unique). With each individual having their own associated reference animal data (including reference contextual data) and historical reference insights, historical primary insights, and reference predictive indicators, the system can analyze and compare the incoming sensor-based animal data and contextual data to identify one or more patterns, rhythms, trends, features, measurements, outliers, abnormalities, anomalies, characteristics/attributes, or the like and create, modify, or enhance one or more predictive indicators.

In a refinement, the creation, modification, or enhancement of the animal data or its one or more derivatives utilizes at least a portion of artificial data. Artificial data can be derived from one or more simulated events, concepts, objects, or systems, and can be generated using one or more statistical models or Artificial Intelligence techniques. In a variation, artificial data can be used to assess one or more biological phenomena (e.g., occurrences that happen in the individual's body such as the beating/rhythms of their heart, oxygenation levels, and the like) of participants in a simulation, with the simulation being operable to enable the modification of one or more variables in order to generate simulated data with desired conditions (e.g., generating a specific type of animal data when the individual is participating in a specific activity in specific environmental conditions with specific medical conditions associated with the individual). Advantageously, artificial data can be used to predict one or more future biological outcomes for any given targeted individual based upon one or more characteristics related to the targeted individual, the one or more sensors, the animal data (e.g., including other metadata such as the activity in which the animal data was collected or other contextual data), or other non-animal data (e.g., other contextual data). In this regard, the artificial data can be utilized as a baseline for any given individual to compare current animal data readings (and insights and indicators derived from it) with predicted readings. Artificial data can be incorporated as part of the reference animal data to derive the at least one reference insight or predictive indicator, and/or as part of the one or more animal data sets gathered from the one or more source sensors to derive the at least one primary insight or predictive indicator.

In another refinement, the at least one reference insight or predictive indicator is created, modified, or enhanced using one or more Artificial Intelligence techniques based upon a subject's one or more biological patterns, rhythms, trends, features, measurements, outliers, abnormalities, anomalies, characteristics/attributes, or the like derived from one or more types of animal data. In this refinement, the system can leverage the one or more Artificial Intelligence techniques to enhance or predict what the subject's body will do in one or more modeled scenarios and create, modify, or enhance one or more reference insights or predictive indicators in order to compare historical and existing animal data with the subject's future animal data at any given point in time. For example, in the course of a tennis match, the system can run one or more simulations to create a reference insight that predicts what the subject's one or more animal data readings should look like in future games or sets that enables the system to create comparisons with the subject at any moment in time based upon their animal data (e.g., the real-time or near real-time animal data gathered) and the corresponding one or more primary insights.

In one variation, simulated animal data is generated by randomly sampling at least a portion of the set of real animal data. In another variation, real data is transformed into simulated data by adding a small random number to each value of a real data set. In this context, small means that the random number has a value within a predetermined percent of the number to which it is added. In a refinement, the predetermined value in preferential order is 1, 10, 20, 30, 40, or 50 percent of the value to which it is added. In a further refinement, the small random number has a mean of zero. In another variation, an offset value is added to each value of real animal data. In a still a further refinement, the offset value in preferential order 0.1, 0.5, 1, 2, 3, 5, or 10 percent of the value to which it is added. For this purpose, the random numbers used for random sampling can be uniformly distributed or normally (e.g., Gaussian random numbers) distributed.

In another variation, one or more simulations can be created on the fly based on past data and learning. In this regard, the simulated animal data can be transformed into a form that can be inputted into a simulation (e.g., a video game, simulated sporting event, simulated event for predicting or forecasting one or more biological-based events for purposes such as placing or accepting a bet) by a number of methods. In one refinement, real animal data is numerically modeled by fitting the real animal data to a function with one or more independent variables or one or more adjustable parameters that are optimized to provide a fit. In this context, such a fitted function is referred to as a model. In such data models, the one or more independent variables or parameters are inputted by the simulation to provide simulated data output. In this regard, time (t) is a useful independent variable that can be used to output a simulated biological output (e.g., physiological output) as a function of time in which a simulated individual is participating in a simulated event. In particular, one or more biological parameters can be associated with a virtual participant in a simulation as a function of time.

In another variation, one or more biological parameters for previously acquired real animal data from one or more targeted subjects can be approximated by a probability distribution. Examples of probability distributions include, but are not limited to Bernoulli distributions, uniform distributions, binomial distributions, normal distributions (i.e., Gaussian), Poisson distributions, exponential distributions, Lorentzian distributions, and the like. Typically, these probability distributions can be randomly sampled to assign one or more biological parameters (e.g., physiological parameters, biomechanical-based parameters, location-based parameters, genetic-based parameters) to one or more simulated participants in a simulation. For example, biological parameters for previously acquired real animal data from one or more targeted subjects can be approximated by a gaussian distribution with the mean and standard deviation as adjustable parameters. The Gaussian distribution can then be randomly sampled to provide values for a simulation. Alternatively, the real animal data can be fit to any function (e.g., a line, polynomials, exponential, Lorentzian, piecewise linear or a spline between real data points, and the like) which is then applied by a simulation. In a refinement, the previously acquired real animal data can have one or more extrinsically associated parameters (e.g., contextual data) such as temperature, humidity, elevation, time, and other non-biological data, which can be applied as an independent variable or parameter in the one or more simulations. In another refinement, one or more biological parameters (e.g., heart rate, HRV, diastolic blood pressure, systolic blood pressure, perspiration rate, distance run, etc.) for a specified targeted individual can be, as a function of time while engaged in an activity, functionally modeled (e.g., fit to polynomials). In this latter example, a simulation can use the modelled function to provide values for the targeted individual as the simulation progresses in time. In this regard, the simulated data can be used to assess biological phenomena (e.g., occurrences that happen in the individual's body such as the beating/rhythms of their heart, oxygenation levels, and the like; or a combination of biological phenomena from which an insight can be derived such as fatigue) of participants in a simulation. For example, the running total for the amount of time a player has an elevated heart rate, diastolic blood pressure, systolic blood pressure, perspiration rate can be used as a measure of fatigue.

In a variation, artificial data sets can be generated, either randomly or otherwise, subject to one or more initiation parameters set by the user. This can be useful in the event real animal data a user desires cannot be acquired, captured, or created in a requested timeframe or manner. In the case where a user has requirements that may not make it feasible to acquire real animal data, prediction system 10 may create artificial animal data derived from at least a portion of real animal data or one or more derivatives thereof that conforms to the parameters established by the user, which can be made available for consumption (e.g., the one or more outputs of the one or more simulations can be made available for purchase or acquisition). In this regard, the one or more parameters the data acquirer selects determines the scope of relevant real animal data that can be utilized as one or more inputs upon which the artificial data is generated, and/or to ensure that the artificial output generated meets the requirements desired by the acquirer. For example, a sports betting operator may request a predictive indicator for a sports match taking place in specific conditions not previously collected. Prediction system 10 may have, for example, data sets from similar matches in different conditions and/or different matches in similar conditions, so the simulation system can create the simulated data sets in order to generate one or more predictive indicators to fulfill the request. To create the requested data sets, the simulation system may use the required parameters and randomly generate the artificial data sets (e.g., artificial ECG data sets) based on the real sets of real animal data. The new one or more artificial data sets can be created by application of one or more Artificial Intelligence techniques that will analyze previously captured data sets that match some or all of the characteristics required by the acquirer. The one or more Artificial Intelligence techniques (e.g., one or more trained neural networks, Machine Learning models) can recognize patterns in real data sets, be trained by the collected data to understand animal (e.g., human) biology and related profiles, be further trained by collected data to understand the impact of one or more parameters or variables on animal biology and related profiles, be further trained by collected data to understand the impact of one or more parameters or variables on animal biology and related profiles for a specific individual or subset of individuals, and create artificial data that factors in the one or more parameters or variables chosen by the acquirer in order to match or meet the minimum requirements of the acquirer. In a refinement, dissimilar data sets from similar individuals, or similar data sets from dissimilar individuals may also be utilized by the one or more Artificial Intelligence models for both model training and data generation purposes. In another refinement, a user chooses one or more parameters or variables for one or more simulations that utilize at least a portion of animal data, one or more simulations occur, and one or more users acquire at least a portion of the simulated data or one or more derivatives thereof for consideration (e.g., payment, other non-monetary value). For example, in the context of sports betting, the simulation system can be operable to offer bettors, bookmakers, or other relevant parties with an opportunity to acquire (e.g., purchase) one or more simulations utilizing at least a portion of collected animal data (e.g., the collected athlete sensor data) in order to predict one or more outcomes. Advantageously, such simulations can occur in real-time or near real-time. In another refinement, at least a portion of non-animal data is utilized as one or more parameters or variables in the one or more simulations.

In another refinement, simulated data can be created by the application of one or more Artificial Intelligence techniques (e.g., which for definition purposes can include Machine Learning, Deep Learning, and Statistical Learning-based models, methods, and the like) which can, for example, utilize one or more neural networks to analyze one or more previously captured or created data sets that match at least one of the characteristics required by the acquirer, the details of which are described herein. In this regard, the Artificial Intelligence-based engine recognizes one or more patterns or upper and lower limits in what is possible for a variety of scenarios in one or more real animal data sets and creates artificial data that matches or meets the minimum requirements of the user (e.g., the wagering entity, bettor, a pharmaceutical or healthcare provider seeking to acquire large amounts of data with specific characteristics, an insurance provider, etc.). The one or more data sets can be created based on a single individual, a group of one or more individuals with one or more similar characteristics, a random selection of one or more individuals within a defined group of one or more characteristics, a random selection of one or more characteristics within a defined group of one or more individuals, a defined selection of one or more individuals within a defined group of one or more characteristics, or a defined selection of one or more characteristics within a defined group of one or more individuals. In a refinement, a group can include a plurality of groups. Based on the user's requirements, the simulation system can isolate a single variable/parameter or multiple variables/parameters for repeatability in creating one or more artificial data sets in order to keep the data both relevant and random.

In another refinement, the one or more Artificial Intelligence techniques includes the use of one or more trained neural networks. In general, a neural network generates simulated animal data after being trained with real animal data and contextual data. Animal data is collected from one or more sensors from one or more target individuals typically as a time series of observations. Sequence prediction Machine Learning algorithms can be applied to predict possible animal data values based on collected data. The collected animal data values and associated contextual data values will be passed on to one or more models during the training phase of the neural network. The neural network utilized to model this non-linear data set will train itself based on established principles of the one or more neural networks. In a further refinement, the one or more trained neural networks utilized to create, modify, or enhance the at least one reference insight, primary insight and/or predictive indicator consists of one or more of the following types of neural networks: Feedforward, Perceptron, Deep Feedforward, Radial Basis Network, Gated Recurrent Unit, Autoencoder (AE), Variational AE, Denoising AE, Sparse AE, Markov Chain, Hopfield Network, Boltzmann Machine, Restricted BM, Deep Belief Network, Deep Convolutional Network, Deconvolutional Network, Deep Convolutional Inverse Graphics Network, Liquid State Machine, Extreme Learning Machine, Echo State Network, Deep Residual Network, Kohenen Network, Support Vector Machine, Neural Turing Machine, Group Method of Data Handling, Probabilistic, Time delay, Convolutional, Deep Stacking Network, General Regression Neural Network, Self-Organizing Map, Learning Vector Quantization, Simple Recurrent, Reservoir Computing, Echo State, Bi-Directional, Hierarchal, Stochastic, Genetic Scale, Modular, Committee of Machines, Associative, Physical, Instantaneously Trained, Spiking, Regulatory Feedback, Neocognitron, Compound Hierarchical-Deep Models, Deep Predictive Coding Network, Multilayer Kernel Machine, Dynamic, Cascading, Neuro-Fuzzy, Compositional Pattern-Producing, Memory Networks, One-shot Associative Memory, Hierarchical Temporal Memory, Holographic Associative Memory, Semantic Hashing, Pointer Networks, Encoder-Decoder Network, Recurrent Neural Network, Long Short-Term Memory Recurrent Neural Network, or Generative Adversarial Network. Additional details related to a system for generating simulated animal data models are disclosed in U.S. patent Ser. No. 17/251,092, filed on Dec. 10, 2020 with a priority date of Sep. 6, 2019; the entire disclosure of which is hereby incorporated by reference.

In another refinement, the system generates one or more artificial data values when detecting and replacing one or more outlier or missing values generated from one or more sensors in order to enable one or more calculations, computations, combinations, predictions, probabilities, possibilities, estimations, evaluations, inferences, determinations, deductions, observations, or forecasts related to the at least one primary insight, reference insight, or predictive indicator. In many cases, the one or more sensors produce measurements that are provided to a server, with the sensor or server applying methods or techniques to filter the data and generate one or more animal data values (e.g., ECG values, HRV values). However, in cases where data has an extremely low signal-to-noise ratio, or in some cases when one or more values are missing, pre-filter logic can be required to generate artificial data values. In one aspect, a pre-filter method whereby the system takes a number of steps to “fix” the data generated from the sensor to ensure that the one or more data values generated are clean and fit within a predetermined range is proposed. The pre-filter logic would ingest the data from the sensor, detect any outlier values, “bad” values, or missing values, and replace these values with expected or “good” artificial values and pass along the “good” artificial values as its computation of the one or more animal data values (e.g., heart rate values). The term “fix” refers to an ability to create one or more alternative data values (i.e., “good” values) to replace values that may fall out of a preestablished threshold or missing values with the one or more “good” data values aligning in the time series of generated values and fitting within a preestablished threshold. These steps would occur prior to any actions taken upon the received animal data to calculate the one or more animal data values (e.g., heart rate or HRV values). Advantageously, the pre-filter logic and methodology for identification and replacement of one or more data values can be applied to any type of sensor data collected, including both raw and processed outputs. Additional details related to a system that generates artificial animal data values are disclosed in U.S. patent Ser. No. 16/776,696 filed on Sep. 2, 2020 with a priority date of Jan. 14, 2019; the entire disclosure of which is hereby incorporated by reference.

In another refinement, the system can be configured to evaluate the incoming sensor-based animal data in conjunction with the contextual data (e.g., such as the one or more variables and other metadata, which may include other animal and/or non-animal data) and correlate the gathered information with the outcome of one or more events or sub-events. Characteristically, the system can be configured to utilize one or more Artificial Intelligence-based techniques (e.g., AI techniques, models, methods, and the like) to identify one or more patterns, rhythms, trends, features, measurements, outliers, abnormalities, anomalies, characteristics/attributes, and the like derived from, at least in part, the sensor-based animal data from a targeted individual or group (e.g., subset) of targeted individuals that are associated with a specific outcome for any definable event. The one or more patterns, rhythms, trends, features, measurements, outliers, abnormalities, anomalies, characteristics/attributes, and the like—including one or more changes or variations in the animal data based upon the one or more variables that are associated with the specific outcome (e.g., patterns/trends that occurred resulting in the outcome, such as a point win/loss in a sports competition) in the context of the definable event—can be unique to each individual or unique to a subset of individuals. This enables the system to compare the animal data and its associated contextual data with other animal data sets that produce similar or dissimilar outcomes in similarly defined events and scenarios (e.g., in some variations, non-similarly defined events and scenarios) both on an individual basis and across individuals. Advantageously, depending on the data being collected by the system from the one or more source sensors, the system can be configured to identify the one or more types of animal data being collected in real-time or near real-time from the targeted subject by the system as well as associated contextual data, and gather reference animal data and reference contextual data that matches the data being collected—at least in part—in order to generate one or more insights (e.g., reference insights) based on available data that enable comparison with the real-time or near real-time information.

In some variations, the use of one or more Artificial Intelligence techniques enables the AI to create an individualized understanding of the subject's body and its associated biological functions based upon animal data (e.g., create a digital map of biological-based functions or responses from the animal body associated with contextual data and one or more specific outcomes that is unique and specific to an individual or a subset of individuals in that given context) in order to create one or more insights based upon that data. For example, by utilizing one or more Artificial Intelligence techniques, the system can analyze both reference animal data and the real-time or near real-time animal data from the one or more source sensors to create, modify, enhance, or access one or more reference and primary insights that enable one or more predictive indicators to be created, modified, or enhanced. The system can be further trained to observe the one or more real-world event outcomes and compare the real-world event outcome to the prediction made by the system, taking into the account the one or more reference insights and primary insights used to create, modify, or enhance the prediction. This enables the system to test the efficacy of its prediction and learn from the actual observed event outcome in order to enhance the prediction accuracy, reliability, and repeatability of future predictive indicators (e.g., the system can determine whether the reference insight and primary insight led to making the correct prediction and make one or more modifications, if required, to the type of insight it creates, the type of animal data and/or contextual data it incorporates into the one or more insights, the type of Identifier it uses to create the insight, the weight given to one or more variables or animal data, and the like in order to make a more accurate prediction in the future). In this scenario, the system may also be configured to create, modify (e.g., adjust), or enhance one or more weights—in the context of the event and the one or more observed event outcomes—given to at least a portion of the animal data, reference animal data, the one or more variables associated with the animal data and/or the reference animal data, or a combination thereof, based upon the observation of the one or more real-world outcomes. Characteristically, this setup enables new predictive indicators (e.g., predictions) to be created, modified, or enhanced dynamically and in real-time or near real-time as new, real-time or near real-time animal data is gathered by the system from the one or more source sensors. In a refinement, by utilizing one or more Artificial Intelligence techniques such as Machine Learning, Statistical Learning, or Deep Learning techniques, one or more neural networks can be trained with gathered reference animal data and contextual data (e.g., including event data and its associated outcome data) to understand the one or more biological functions of a targeted individual and how one or more variables affect any given biological function. The system can be further trained to understand what outcome (or outcomes) occurred based on the one or more biological functions and the impact of the one or more variables, enabling correlative and causative analysis. For example, upon being trained to understand information such as the one or more biological functions of a targeted athlete within any given scenario including a current existing scenario (e.g., a point in time in a current event), the one or more variables that may impact the one or more biological functions of the targeted athlete within any given scenario including the current existing scenario, contextual data associated with the one or more biological functions of the targeted athlete within any given scenario including the current existing scenario, the one or more outcomes that have previously occurred in any given scenario including the current existing scenario based on the one or more biological functions exhibited by of the targeted athlete and/or the one or more variables present, the one or more biological functions of athletes similar and dissimilar to the targeted athlete in any given scenario including scenarios similar to the current existing scenario, the one or more other variables that may impact the one or more biological functions of the targeted athlete in any given scenario including scenarios similar to the current existing scenario, the one or more variables that may impact the one or more biological functions of other athletes similar and dissimilar to the targeted athlete in any given scenario including scenarios similar to the current existing scenario, the one or more outcomes that have previously occurred in any given scenario including scenarios similar to the current existing scenario based on the one or more biological functions exhibited by athletes similar and dissimilar to targeted athlete and/or the one or more variables, and contextual data associated with these scenarios, the system can be configured to generate one or more reference insights related to the targeted athlete for any given context during an event. Additional details related to an animal data prediction system are disclosed in U.S. patent Ser. No. 16/977,278, filed on Sep. 1, 2020 with a priority date of Apr. 15, 2019; the entire disclosure of which is hereby incorporated by reference. In a refinement, the system runs one or more simulations to create, modify, or enhance the one or more reference insights. In another refinement, the system runs one or more simulations utilizing the reference animal data, the reference contextual data, the animal data derived from the one or more source sensors, and the contextual data to create, modify, or enhance one or more predictive indicators.

In one variation, the one or more reference insights can be created, modified, enhanced, or accessed at the direction of the system on the fly (e.g., dynamically) based upon the type of animal data being gathered by the system from the one or more source sensors, the types of source sensors being utilized (e.g., and their one or more associated operating parameters), other contextual data (e.g., the event, the point in time within the event, the environment, momentum, and the like), the one or more Identifiers (e.g., the system is configured to understand what one or more outcomes occur when any given pattern, anomaly, trend, or the like happens in the individual's animal data or across individuals with one or more similar characteristics or individuals in similar environments or scenarios), the one or more primary insights, or a combination thereof. For example, in the event the system is programmed to create a predictive indicator for a win/loss match outcome in a current match featuring the targeted individual, the system can access all or a subset of relevant data from the reference animal database based upon the current data being collected to create the one or more reference insights. In a variation, the context of a given event may trigger the system to access one or more reference insights on the fly that have been already created and made accessible for comparison purposes by the system. In another variation, the system can be configured to generate one or more insights despite missing at least a portion of information (e.g., the reference animal database may have animal data sets associated with outcomes for a specific individual that feature specific contextual/variable data such as environmental data but the animal data being gathered from the one or more source sensors does not include any associated environmental data. However, the system can be configured to generate the one or more insights to make the one or more predictions despite the missing data. In some variations, the system may run one or more simulations to fill in missing data with artificial data). In another variation, the one or more computing devices create, modify, or enhance at least one reference insight from animal data that is available as both reference animal data and animal data gathered by the one or more source sensors from the targeted subject. For example, if a respiration rate-based sensor is not being used by the targeted subject, then the system will not create or access a reference insight that incorporates respiration rate. This methodology may also be applied to reference contextual data and contextual data captured with the animal data. In another variation, the one or more computing devices selectively use (e.g., select and use) a subset of one or more reference insights from the reference animal data such that the subset can be compared against the one or more primary insights derived from animal data that can be captured by the one or more source sensors from the targeted subject. In another variation, one or more primary insights are created, modified, or enhanced at the direction of the system on the fly (e.g., dynamically) based upon the one or more reference insights, the type of animal data being gathered by the system from the one or more source sensors, the types of source sensors being utilized, other contextual data, the one or more Identifiers, or a combination thereof. In another variation, the one or more computing devices selectively use a subset of animal data from the reference animal data such that the subset can be compared against the animal data that can be captured by the one or more source sensors from the targeted subject.

In another variation, by utilizing one or more Artificial Intelligence techniques such as Machine Learning, Statistical Learning, or Deep Learning techniques, the system can identify one or more patterns, rhythms, trends, features, measurements, outliers, abnormalities, anomalies, characteristics/attributes, and the like in the reference animal data that can be correlated with specific outcomes based upon the context in which the animal data was collected. Such Identifiers can be unique to each individual when compared to other individuals, unique across a subset of individuals, or applicable to all individuals. With each individual having at least one reference insight for each potential outcome in any given scenario based upon the reference animal data, the system can be configured analyze the incoming sensor-based animal data (e.g., in conjunction with the one or more variables and other metadata, which may include other animal and/or non-animal data) to create one or more primary insights that enable comparison with the one or more reference insights. Advantageously, depending on the data being collected by the system from the one or more source sensors, the system can be operable to identify the one or more types of animal data currently being collected from the targeted subject by the system via the one or more source sensors, and create or access a reference insight only based on the data currently being collected by the system via the one or more source sensors and any other systems collecting contextual data, thus enabling one or more predictive indicators to be created, modified, or enhanced based upon available data. In a refinement, the one or more primary insights, reference insights, and predictive indicators are associated with the real-world observed event outcome and included as part of the reference animal database (e.g., as historical reference insights 176, historical primary insights 172, and as reference predictive indicators 174) and used as training data for the AI to enhance accuracy, reliability, and repeatability of the predictive indicator for one or more future events. In a variation, the one or more computing devices create, modify, enhance, or access the one or more reference insights and primary insights from animal data that is available as both reference animal data and animal data gathered by the one or more source sensors from the targeted subject. For example, if a hydration-based sensor is not being used by the targeted subject, then the system will not create an insight utilizing hydration-based data. In a variation, the one or more computing devices selectively use a subset of the one or more reference insights from the reference animal data such that the subset can be compared against the one or more primary insights derived from animal data that can be captured by the one or more source sensors from the targeted subject. In another variation, the one or more computing devices selectively use a subset of animal data from the reference animal data such that the subset can be compared against the animal data that can be captured by the one or more source sensors from the targeted subject. In a refinement, the system can be programmed to identify one or more Identifiers, or seek and identify previously unidentified Identifiers (e.g., patterns) based upon established AI techniques, and train an AI to correlate the one or more Identifiers with one or more event outcomes on the fly (i.e., dynamically) that enable the system to dynamically (1) create, modify, enhance, or access one or more reference insights; (2) create, modify, or enhance one or more primary insights; (3) create, modify, or enhance one or more predictive indicators; or (4) a combination thereof.

In a refinement, the one or more outputs of prediction system 10 or the methods associated with prediction system 10 are used either directly or indirectly: (1) to create, modify, enhance, evaluate, or communicate one or more odds; (2) to create, modify, enhance, evaluate, or communicate one or more bets; (3) as a market upon which one or more bets are placed or accepted; (4) to formulate one or more strategies; (5) to create, modify, enhance, acquire, offer, or distribute one or more products; (6) to mitigate, prevent, or take one or more risks; (7) to recommend one or more actions; (8) as one or more signals or readings utilized in one or more simulations, computations, or analyses; (9) as part of one or more simulations, an output of which directly or indirectly engages with one or more users; (10) as one or more core components or supplements to one or more mediums of consumption; (11) in one or more promotions; or (12) a combination thereof.

In a variation with respect to applications (1), (2) and (3), the one or more odds can include one or more predictions, probabilities, or possibilities related to a future outcome or occurrence, with one or more predictions, probabilities, or possibilities connected. In addition, “communication” can include visualization of the one or more predictions, probabilities, or possibilities (e.g., displaying a probability via an application, displaying an output-based probability for a targeted individual within an augmented reality or virtual reality system), verbal communication of one or more predictions, probabilities, or possibilities (e.g., a voice-activated virtual assistant that informs a targeted individual of the likelihood of a possible outcome), and the like. Lastly, modification of a prediction, probability, or possibility can include revising a previously determined prediction, probability, or possibility for an event. Furthermore, a market can be a specific type or category of bet or wager on a particular event (e.g., a sporting event, a health or medical event, a simulated event), including one or more sub-events. A market can be created and offered or leveraged for any event or sub-event. Oftentimes, organizations that accept one or more bets offer a plurality of betting markets on each event or sub-event, with odds listed for each market. Specific types of bets can include a proposition bet (“prop bet”), spread bet, a line bet, a future bet, a parlay bet, a round-robin bet, a handicap bet, an over/under bet, a full cover bet, an accumulator bet, an outright bet, a teaser bet, and the like. In a refinement, a market or wager includes at least one of a proposition bet, spread bet, a line bet (moneyline bet), a future bet, a parlay bet, a round-robin bet, a handicap bet, an over/under bet, a full cover bet, an accumulator bet, an outright bet, or a teaser bet. In another refinement, the one or more bets or betting products (e.g., products developed to inform a bettor prior to placing a bet) are dynamically created, modified, or enhanced by the computing subsystem based upon the one or more reference insights, primary insights, predictive indicators, the animal data, the contextual data, or a combination thereof, which may occur utilizing one or more Artificial Intelligence techniques. In addition, acceptance of a wager can be, for example, acceptance of a bet by a system utilizing the one or more outputs (e.g., a bet type utilizing a predictive indicator). In another refinement, the one or more odds are dynamically created, modified, or enhanced by the computing subsystem for one or more events or sub-events based upon the one or more reference insights, primary insights, predictive indicators, the animal data, the contextual data, or a combination thereof, which may occur utilizing one or more Artificial Intelligence techniques. In another refinement, the one or more odds created, modified, enhanced, or evaluated dynamically and in real-time or near real-time as new animal data, contextual data, or a combination thereof is gathered by the system, which may occur directly (e.g., via the computing subsystem) or indirectly (e.g., via a cloud server associated with the computing subsystem or other computing device).

Characteristically, prediction system 10 can operate as—or as part of—a sportsbook or other platform where monetary gains or losses can result, at least in part, from one or more outcomes (e.g., other wagering platforms, platforms of chance, and/or skill including platforms that offer games of luck and/or skill, and the like), or as a provider of one or more products or services related to the one or more outputs of prediction system 10 to one or more sportsbooks or other platforms. In a refinement, prediction system 10 can provide one or more products or services related to its one or more outputs to one or more individuals, or groups of individuals (e.g., including companies, entities, and the like), that interact with one or more sportsbooks or other platforms.

In a variation with respect to application (4), a strategy can include any strategy that uses at least a portion of the one or more outputs either directly or indirectly. For example, a strategy can be a plan of action to determine whether or not to place a bet, whether or not to take a specific action related to the one or more outputs (e.g., predictive indicator), in-game performance strategies (e.g., whether to play or sit an athlete during a game), and the like. A strategy can also include a complete trading/betting strategy that is based on the running of one or more simulations and simulated data, or derived from the one or more predictive indicators which may derived from other methods, to predict potential outcomes and thresholds upon which the predefined rules will action against. In addition, the one or more outputs or one or more derivatives thereof can be utilized in one or more further calculations, computations, combinations, measurements, derivations, extractions, extrapolations, simulations, creations, modifications, enhancements, estimations, evaluations, inferences, establishments, determinations, conversions, deductions, observations, or communications related to the formulation of one or more strategies. In this context, the term “formulation” can include of one or more modifications, enhancements, and the like.

In a variation with respect to application (5), one or more products can be one or more goods or services that are designed to be distributed or sold. A product can be any product in any industry or vertical that can be created, modified, enhanced, offered, or distributed, so long as the product uses at least a portion of the one or more animal data-based outputs either directly or indirectly. For example, a product can be a market upon which one or more wagers are placed or accepted (e.g., a bet type), a product for coaches or employers related to their team or employees (e.g., animal data-based dashboard for sports coaches, and trainers displaying real-time health metrics), and the like. In a refinement, at least a portion of the simulated data, the one or more predictive indicators, reference insights, primary insights, animal data, contextual data, or a combination thereof, are used to create, modify, enhance, offer, acquire, accept, or distribute at least one of: a proposition bet, a spread bet, a line bet, a futures bet, a parlay bet, a round-robin bet, a handicap bet, an over/under bet, a full cover bet, an accumulator bet, an outright bet, a teaser bet, or a combination thereof. It is inclusive of the one or more outputs or its one or more derivatives thereof leading to (or resulting in) the creation of a product. For example, a product can be the output itself (e.g., purchasing the one or more data outputs as a data set), a health application that displays the one or more outputs, a suite of algorithms designed to provide a particular insight/prediction related to a subject based upon the output, a sports betting application which provides one or more bets or betting-related products (e.g., data, analytics, visualization tools, betting insurance underwriting tools, match simulator, and the like), which can be operated as part prediction system 10 (e.g., via one or more computing devices 42) or can be operated separately from prediction system 10 (e.g., prediction system 10 provides the one or more outputs to one or more sports betting applications or systems via computing devices 42 not operated by prediction system 10), a consumer product that utilizes the one or more outputs (e.g., beverages such as isotonic drinks that utilize the one or more outputs to personalize ingredients based upon a subject's biological information, foods), and the like. For clarification purposes, “enhance” can also include “to be part of” a product should the enhancement add value. In addition, and in many cases, “create” can be inclusive of “derive” and vice versa. Similarly, “create” can be inclusive of “generate” and vice versa. In addition, “offer” can be inclusive of “provide.” Lastly, an “acquirer” of a product could be, for example, a consumer, an organization, another system, any other end point that could consume or receive the product, and the like.

In a variation with respect to application (6), mitigation or prevention of risk can include any action, non-action, strategy, recommendation, reclassification of risk, changing of a risk profile, and the like related to reducing or preventing risk (e.g., wagering risk). In a refinement, it can also include taking additional risk.

In a variation with respect to application (7), to recommend one or more actions includes both a recommendation that is inferred by the one or more outputs either directly or indirectly (e.g., a predictive indicator derived from the reference animal data, animal data, contextual data, simulated data, or a combination thereof, that provides a probability of an occurrence happening may infer an action to be taken) as well as a recommendation directly stated based on the one or more outputs (e.g., a recommendation that an action be taken based on a predictive indicator derived from one or more simulations that provide the probability of an occurrence happening or a prediction). In a refinement, a recommendation can be comprised of a plurality of recommendations. In a refinement, the one or more outputs of prediction system 10 are used to take one or more actions. An action can be any action that is directly or indirectly related to at least a portion of one or more outputs. An action includes an action that is derived from (or results from) the one or more outputs. It can be, for example, an action to prevent a predicted outcome from occurring based upon the one or more outputs or an action enable a predicted outcome to occur. The action can also be, for example, an action to place a wager (e.g., the athlete's predicted to win the next game based upon the predictive indicator, therefore a user places a bet), an action to take a specific action (e.g., a system communicating an action to take a specific action such as “place a bet.” “avoid x activity,” an action to take no action at all, and the like.

In a variation with respect to application (8), a signal or reading can include any form or a signal or reading, as well as any format related to the information (e.g., including as one or more data sets).

In a variation with respect to application (9), a simulation includes both the production of one or more computer models, as well as imitation of one or more situations or processes. Simulations have a wide range of engagement uses, including simulations that are utilized to generate the one or more outputs, which any use of the outputs can be considered either direct or indirect engagement, as well as inclusion of the one or more outputs within one or more simulations, which may engage one or more users (e.g., a video game or other game-based or virtual environment system, an augmented reality or virtual reality system, a mixed reality system, virtual game play system, and the like).

In a variation with respect to application (10), the one or more mediums of user consumption can be any medium where a user can directly or indirectly consume the one or more outputs. A medium can include, for example, a health monitoring application (e.g., remote monitoring platform) that communicates a heart status check via the one or more outputs, a remote rehabilitation or telehealth platform that communicates the one or more outputs to the platform during an activity (e.g., remote exercise, virtual doctor visit) while enabling the remote medical professional or rehabilitation specialist to see the patient via an integrated video display, an insurance application that communicates an insurance adjustment based at least in part from the one or more outputs, a sports wagering platform utilizing the one or more outputs, a human performance application, a fan engagement platform, and the like. It can also include a media broadcast that incorporates the one or more outputs (e.g., providing a prediction related to the outcome of a sporting event), a sports streaming content platform (e.g., video platform) that integrates the one or more prediction system outputs as a supplement to the live sports event being watched (e.g., enabling a user to place a wager while watching the live content), and the like. It can also include non-display mediums (e.g., a key fob or scannable object) that provides information related to the health status of one or more individuals to one or more other systems.

In a variation with respect to application (11), the one or more promotions can be any promotion that provides support in furtherance of the acceptance and/or acquisition (e.g., sale, distribution) of one or more products derived at least in part from the animal data or its one or more derivatives. This includes one or more advertisements, an offer that uses the one or more outputs (e.g., an offer to a subject to obtain a specific bet type or odds related to a bet utilizing the one or more outputs), a discounting mechanism that uses the one or more outputs (e.g., the predictive indicator indicates that player X will has a n percent chance to lose the match vs player Y; therefore, the prediction system or a system in communication with the prediction system will provide the user/bettor with more or less favorable odds for player X to win the match, with updates to the odds occurring in real-time or near real-time based on new information collected by the system), and the like.

In a variation with respect to application (12), “a combination thereof” can include any combination of the aforementioned applications, including all of the aforementioned applications or a subset of the aforementioned applications.

In at least another aspect, a system for generating dynamic real-time predictions using heart rate variability is described. The system includes one or more source sensors that gather animal data as gathered animal data from a targeted individual wherein at least a portion of the gathered animal data is heart rate variability data (e.g., HRV is derived from the animal data; HRV is gathered by the system from the one or more source sensors). Animal data is transmitted by the one or more source sensors electronically. A transmission subsystem provides the transmitted animal data to a computing subsystem, wherein the computing subsystem gathers the animal data in real-time or near real-time. The computing subsystem is further configured to gather contextual data related to the gathered animal data and an event associated with (e.g., featuring, which includes, and the like) the subject. In this context, an event can be a scenario (e.g., when the player is losing 3-2 in the 4th set of a match), a specific point in time (e.g., 3rd game of the 2nd set; 1st point of the 2nd game; first 2 minutes of the 4th quarter), an entire event (e.g., a whole match), a sub-event (e.g., a point within a match), and the like. In a refinement, an event includes any scenario with a quantifiable, definable, observable, and/or measurable outcome. In another refinement, an event is comprised of one or more sub-events. In another refinement, an event includes a plurality of events. The event associated with the targeted individual can be the event in which the targeted individual is participating in, or sub-events within each event (e.g., games or sets within a match; the first n minutes of a game; the first n points of a game; and the like), or the activity the targeted individual is undertaking.

The computing subsystem is configured to take one or more actions (e.g., normalize, timestamp, aggregate, clean, tag, store, manipulate, denoise, process, enhance, organize, categorize, analyze, visualize, simulate, anonymize, synthesize, summarize, replicate, productize, or synchronize the data) with gathered contextual data and the animal data derived, at least in part, from the one or more source sensors to create, modify, or enhance at least one primary insight related to at least one physiological-based condition of the targeted individual, the computing subsystem being further operable to make one or more modifications or enhancements to the at least one primary insight as new information (e.g., data such as additional animal data, additional contextual data, or a combination thereof) is gathered by the computing subsystem. A physiological-based condition refers to one or more data or derived descriptions that can be assigned to a targeted individual that describe a biological status or state of the targeted individual or a biological phenomenon associated with the targeted individual utilizing at least a portion of their animal data. In a variation, a physiological-based condition can be any biological state or phenomena that can be quantified, defined, observed, or measured based upon one or more animal data readings. Characteristically, the system is operable to make one or more modifications or enhancements to the at least one primary insight in real-time or near real-time. In some variations, a rolling window-based method is used.

The computing subsystem is further configured to access at least one reference insight, the at least one reference insight and the at least one primary insight being used to create, modify, or enhance at least one predictive indicator. In this context, “access” can include “gather” and vice versa. In a refinement, “access” can also include create, modify, or enhance. The at least one predictive indicator can be created, modified, or enhanced using one or more statistical or Artificial Intelligence-based methodologies (or a combination thereof). Additionally, the computing device can also access the reference insight from another computing device, or it can access the reference insight as part of the reference animal data (e.g., another computing device created, modified, or enhanced the reference insight and provided the reference insight to the computing device as part of its reference animal data database). Lastly, the at least one predictive indicator is used by one or more computing devices to perform one or more of: (1) create, modify, enhance, or evaluate one or more odds; (2) create, modify, enhance, or evaluate one or more bets; (3) as a market upon which one or more bets are placed or accepted; (4) formulate one or more strategies; (5) create, modify, enhance, acquire, offer, or distribute one or more products; (6) mitigate, prevent, or take one or more risks; or (7) a combination thereof.

In a refinement, the animal data is human data. In another refinement, the computing subsystem utilizes at least one weighted variable to create, modify or enhance the at least one primary insight. A variable can be, for example, animal data, contextual data (e.g., non-animal data, animal data), or a combination thereof. In another refinement, the computing subsystem utilizes at least one weighted variable to create, modify or enhance the at least one reference insight or predictive indicator. In another refinement, the computing subsystem modifies or enhances the at least one reference insight, primary insight, or predictive indicator based upon new animal data or contextual data being gathered the system.

In another refinement, the computing subsystem provides at least one of the animal data, the contextual data, the at least one reference insight, or the at least one primary insight to one or more computing devices to at least one of: (1) create, modify, enhance, or evaluate one or more odds; (2) create, modify, enhance, or evaluate one or more bets; (3) as a market upon which one or more bets are placed or accepted; (4) formulate one or more strategies; (5) create, modify, enhance, acquire, offer, or distribute one or more products; or (6) mitigate, prevent, or take one or more risks. In a further refinement, the computing subsystem further provides at least one of reference animal data, reference contextual data, the at least one historical reference insight, reference predictive indicator, or the at least one historical primary insight to one or more computing devices to at least one of: (1) create, modify, enhance, or evaluate one or more odds; (2) create, modify, enhance, or evaluate one or more bets; (3) as a market upon which one or more bets are placed or accepted; (4) formulate one or more strategies; (5) create, modify, enhance, acquire, offer, or distribute one or more products; or (6) mitigate, prevent, or take one or more risks. In another refinement, the at least one predictive indicator, the animal data, contextual data, at least one reference insight, or at least one primary insight, or a combination thereof, are further used to (1) to recommend one or more actions; (2) as one or more signals or readings utilized in one or more simulations, computations, or analyses; (3) as part of one or more simulations, an output of which directly or indirectly engages with one or more users; (4) as one or more core components or supplements to one or more mediums of consumption; (5) in one or more promotions; or (6) a combination thereof.

In another refinement, at least one predictive indicator, animal data, contextual data, at least one reference insight, at least one primary insight, or a combination thereof, are distributed to one or more computing devices for consideration. In this context, distributed includes “provided,” “made available.” and the like. In a further refinement, consideration is provided as part of a marketplace for animal data.

In another refinement, the reference animal data, reference contextual data, the at least one historical reference insight, the at least one reference predictive indicator, or the at least one historical primary insight are distributed to one or more computing devices for consideration. Additional details related to a system and method for monetizing animal data are disclosed in U.S. patent Ser. No. 16/977,454 filed on Nov. 5, 2020 with a priority date of Apr. 15, 2019; the entire disclosure of which is hereby incorporated by reference.

In another refinement, the at least one reference insight, primary insight, or predictive indicator related to the targeted subject is created, modified, or enhanced based upon at least one of: a reference insight, a primary insight, a predictive indicator, or animal data related to (e.g., inclusive of “derived from”) another one or more individuals. For example, the system may use the reference indicator from another one or more subjects to adjust the reference indicator of the targeted subject.

In another refinement, the least one reference insight, primary insight, or predictive indicator is communicated to a user prior to placing a bet. Communication can occur via a display device using one or more methods described herein.

In another refinement, the at least one reference insight, primary insight, or predictive indicator is dynamically created, modified, or enhanced by the computing subsystem using one or more Artificial Intelligence techniques.

In another refinement, prediction system 10 operates at least one application or program that enables use of the at least one predictive indicator the animal data, contextual data, at least one reference insight, at least one primary insight, or a combination thereof, to either directly or indirectly: (1) create, modify, enhance, evaluate, or communicate one or more odds; (2) create, modify, enhance, evaluate, or communicate one or more bets; (3) as a market upon which one or more bets are placed or accepted; (4) formulate one or more strategies; (5) create, modify, enhance, acquire, offer, or distribute one or more products; (6) mitigate, prevent, or take one or more risks; (7) recommend one or more actions; (8) as one or more signals or readings utilized in one or more simulations, computations, or analyses; (9) as part of one or more simulations, an output of which directly or indirectly engages with one or more users; (10) as one or more core components or supplements to one or more mediums of consumption; (11) in one or more promotions; or (12) a combination thereof. For example, prediction system 10 can operate as an integrated animal data collection, analysis and distribution platform and sports betting operator.

In another refinement, prediction system 10 provides the at least one predictive indicator, the animal data, contextual data, at least one reference insight, or at least one primary insight to one or more computing devices that operate one or more applications or programs to either directly or indirectly: (1) create, modify, enhance, evaluate, or communicate one or more odds; (2) create, modify, enhance, evaluate, or communicate one or more bets; (3) as a market upon which one or more bets are placed or accepted; (4) formulate one or more strategies; (5) create, modify, enhance, acquire, offer, or distribute one or more products; (6) mitigate, prevent, or take one or more risks; (7) recommend one or more actions; (8) as one or more signals or readings utilized in one or more simulations, computations, or analyses; (9) as part of one or more simulations, an output of which directly or indirectly engages with one or more users; (10) as one or more core components or supplements to one or more mediums of consumption; (11) in one or more promotions; or (12) a combination thereof. The one or more computing devices can be one or more sports betting operators. In some variations, the sports betting operator can be, for example, a sports league, tour, federation, governing body, team, athlete, analytics company, sportsbook operator (e.g., traditional sportsbook operator, rights holder/sports league-owned sportsbook), and the like.

The methods and systems described herein can be applied to a multitude of use cases (e.g., situations, scenarios) where there is a quantifiable, observable, definable, or measurable outcome (e.g., sports, insurance, construction, ecommerce, logistics, healthcare, security such as lie-detection tests or person verification/authentication, and the like) or use cases that aim to identify one or more biological-based patterns, rhythms, trends, features, measurements, outliers, abnormalities, anomalies, characteristics/attributes, and the like, or variations or changes within these identifiers (e.g., anomalies or irregularities based on the context) of one or more individuals (e.g., sports betting integrity system to identify and/or verify one or more patterns or variations in their real-time or near real-time animal data from the one or more individuals as compared to the reference animal data that could indicate match fixing or fraudulent behavior). For example, the one or more methods or systems described herein can be implemented as part of a sensor-based identification and verification system to mitigate or prevent one or more risks, the one or more risks being, for example, the identification of fraudulent biological-based behavior (e.g., identification of intentional biological-based behavior or activity to circumvent an expected outcome, such as behavior to intentionally lose a point, game, or match in a sporting competition). In a variation, prediction system 10 can be implemented as part of an integrity system to identify fraudulent biological-based behavior of individuals based upon their animal data or its one or more derivatives, which can be particularly useful for sports wagering applications whereby the identification of variations in the animal data, inclusive of heart rate variability data, can be implemented to identify match fixing by the system. In this variation, the system predicts one or more outcomes using heart rate variability data, with at least one of the outcomes identifying fraudulent biological-based behavior. In a refinement, the one or more prediction techniques are used as part of a sports wagering/betting integrity indicator to identify fraudulent biological-based behavior. In another refinement, the system predicts one or more outcomes using heart rate variability data, the one or more real-world outcomes being different than the one or more outcomes that the system predicted, at least in part. Based on an analysis of the one or more differences between the one or more real-world outcomes and one or more system predicted outcomes, the reference animal data, the gathered real-time or near real-time animal data, the associated contextual data, or a combination thereof, the system makes a determination that the individual (e.g., athlete) has engaged in fraudulent biological-based behavior. In another refinement, the system runs one or more simulations to make one or more predictions related to one or more outcomes to support, at least in part, the identification of fraudulent behavior. Additional details related to animal data-based identification and recognition systems and methods are disclosed in Pat. No. PCT/US22/26532, filed on Apr. 27, 2022 with a priority date of Apr. 27, 2021; the entire disclosure of which is hereby incorporated by reference.

In at least another aspect, a system for generating dynamic real-time predictions using heart rate variability is described. The system includes one or more sensors that gather animal data from a targeted individual associated with (e.g., prior to, during, after, or a combination thereof) an event wherein at least a portion of the gathered animal data is heart rate variability. The time associated with the data collection is a tunable parameter. Animal data is transmitted by the one or more sensors electronically. A transmission subsystem provides the transmitted animal data to a computing subsystem. A computing subsystem gathers the animal data and contextual data associated with the gathered animal data, the contextual data including event data (e.g., which includes outcomes) associated with the targeted individual. The computing subsystem takes one or more actions to transform the gathered animal data and contextual data into reference data, wherein at least a portion of the reference data is used to create, modify, or enhance one or more features, the one or more features including information derived from heart rate variability data measured over an adjustable time interval, and wherein each of the one or more features is measured in an adjustable time period (e.g., immediately) prior to an outcome associated with the event (e.g., the point, game, match) being determined (e.g., win/loss). In a refinement, the one or more features are the selected data, contextual data, or a combination thereof, that the system uses, in part, to make a prediction. The one or more features can be selected (e.g., defined) by the system. In some variations, one or more AI techniques can be utilized to select the one or more features, at least in part. In another refinement, the one or more features also include information derived from heart rate data. In another refinement, at least a portion of the animal data is grouped as short term, medium term or long term based on the value of time intervals. The computing subsystem organizes (e.g., categorizes) the reference data for the targeted individual by event such that the computing subsystem implements one or more Artificial Intelligence-based models designed and trained with the reference data, the reference data including the one or more features and event data, on an initial subset of one or more events (e.g., games played in a match; points played in a game) associated with the targeted individual from which a predictive indicator for each event outcome is generated and compared against the actual outcomes, wherein the one or more Artificial Intelligence-based models are further tested on a holdout data set derived from at least a portion of the event data on a rolling basis to validate the accuracy of one or more model performances. The size/quantity of the initial subset is a tunable parameter. The number of holdout data sets are also a tunable parameter. In a refinement, the computing subsystem is trained with reference data from one or more other individuals where at least a portion of the reference data from one or more other individuals is utilized to create, modify, or enhance the at least one predictive indicator related to the targeted individual. The computing subsystem is configured to correlate one or more aspects of the targeted individual's reference data, the one or more aspects including their one or more features and the event data (e.g., event outcome data), to create baseline information (e.g., one or more baselines) for the targeted individual.

In a refinement, the system includes one or more source sensors that gather animal data from a targeted individual in real-time or near real-time prior to or during a targeted event (e.g., live event) wherein at least a portion of the gathered animal data is heart rate variability data (or animal data from which heart rate variability can be derived). Animal data that is transmitted by the one or more source sensors electronically. A transmission subsystem provides the transmitted animal data to a computing subsystem. A computing subsystem gathers the animal data and associated contextual data related to the gathered animal data, the associated contextual data including event data from the targeted event (e.g., live event data) associated with the targeted individual. Access to the event data may occur directly or indirectly (e.g., via one or more other computing devices). The computing subsystem is configured to take one or more actions to transform the gathered animal data and the associated contextual data, including the event data from the targeted event, into a data format wherein at least a portion of the transformed data (e.g., animal data, contextual data, targeted event data, or a combination thereof) is used to create, modify, or enhance one or more features, the one or more features including information derived from heart rate variability data measured over an adjustable time interval, and wherein each of the features is measured in an adjustable time period (e.g., immediately) prior to the outcome of the targeted event (e.g., the point, game, match) being determined. In a refinement, the one or more features include at least one of: moving average heart rate over an adjustable time interval, rolling time domain HRV features RMSSD and SDNN over an adjustable time interval, rolling frequency domain features LF, HF, and LF/HF Ratio over an adjustable time interval, or one or more cumulative metrics. In the context of sports and more specifically analyses by individual player, cumulative metrics can include cumulative mean HR by player per event (e.g., point, game, match) or subset of events (e.g., subset of points or games or matches); cumulative maximum HR by player for each event (e.g., point, game, match) or subset of events (e.g., subset of points or games); and the like. In another refinement, the one or more features also include information derived from heart rate data.

In a refinement, the computing subsystem is further configured to take one or more actions to transform the gathered animal data and associated contextual data into reference data, wherein at least a portion of the reference data is used to create, modify, or enhance one or more features, the one or more features including information derived from heart rate variability data measured over an adjustable time interval, and wherein each of the one or more features is measured in an adjustable time period prior to an outcome associated with the event being determined. The computing subsystem accesses the baseline information (e.g., the one or more baselines) for (or related to) the targeted individual prior to each targeted event (e.g., point, game, match), the computing subsystem utilizing the baseline information (e.g., the one or more baselines) and the real-time or near real-time animal data or its one or more derivatives (e.g., the one or more features) to perform one or more calculations to derive a difference in values (e.g., delta). The computing subsystem compares the difference in values to create, modify, or enhance at least one predictive indicator related to the outcome of the targeted event or another (e.g., subsequent, previous) targeted event (e.g., the next point, game, or match). The at least one predictive indicator is used by one or more computing devices (e.g., which can include the computing subsystem, associated computing devices, third party computing devices, and the like) to at least one of (or perform one or more of): (1) create, modify, enhance, or evaluate one or more odds; (2) create, modify, enhance, or evaluate one or more bets; (3) as a market upon which one or more bets are placed or accepted; (4) formulate one or more strategies; (5) create, modify, enhance, acquire, offer, or distribute one or more products; or (6) mitigate, prevent, or take one or more risks.

In a refinement, the computing subsystem is configured to execute each of the one or more steps to create, modify, or enhance the at least one predictive indicator in real-time or near real-time, computing subsystem is configured to execute one or more steps of the one or more methods described herein. In another refinement, the computing subsystem is configured to execute one or more steps (or each of the one or more steps) for a plurality (e.g., group) of targeted individuals, either collectively (e.g., creating a prediction based on a team's collective animal data) or individually, to create, modify, or enhance at least one predictive indicator in real-time or near real-time. In another refinement, the computing subsystem gathers (e.g., accesses) reference data directly or indirectly associated with the targeted individual and/or the targeted event from one or more other computing devices. In another refinement, the computing subsystem does not have access to reference data but instead uses newly-gathered animal data (e.g., collected) from the targeted individual or group or individuals to create the one or more baselines (e.g., which can occur on the fly/dynamically) that are representative of reference data and used for analysis. In another refinement, the computing subsystem uses one or more lagged values for one or more select features, wherein the one or more lagged values range from 1 to any number of seconds or time intervals. In another refinement, the computing subsystem is further operable to calculate one or more additional metrics, the one or more additional metrics including SPIKE_RMSSD_COUNT. In another refinement, one or more performances of the one or more AI-based models are measured by using one or more AI performance measures or indices which include at least one of: confusion matrices, accuracy percentages per event, or distribution of mis-classifications over the spectrum of events. In another refinement, the reference data used to design and train the one or more Artificial Intelligence-based models for each targeted individual or group of targeted individuals is organized by a higher tier in the event hierarchy, a lower tier in the event hierarchy, or a subset of tiers on the event hierarchy. In another refinement, the computing subsystem is trained to learn the one or more correlations between animal data gathered from a plurality of individuals (e.g., including one or more groups of individuals, subsets of individuals) and the associated contextual data from the plurality of individuals, the associated contextual data including the one or more event outcomes. In another refinement, the computing system utilizes the real-time or near real-time animal data, or its one or more derivatives, and the associated contextual data gathered from the targeted individual or a group of targeted individuals, the group of targeted individuals being sourced from the plurality of individuals, to create, modify, or enhance the at least one predictive indicator for the targeted individual or the group of targeted individuals related to one or more outcomes of the targeted event or another one or more events. In a further refinement, the one or more outcomes (e.g., of the targeted event) is a binary outcome. In a further refinement, the one or more outcomes (e.g., of the targeted event) is a multiclass outcome. In another refinement, the at least one predictive indicator is derived utilizing at least one classification algorithm selected from the group consisting of: Random Forest classification algorithm, Random Forest/Decision Trees, Support Vector Machine classifier, K-Nearest Neighbors, Naive Bayes, Linear Discriminant Analysis, Logistic Regression, Neural Networks, or Gradient Boosting Machine Classifier. In another refinement, the predictive indicator is used to create, modify, or enhance one or more bets. In another refinement, the one or more bets include at least one of: a proposition bet, spread bet, a line bet, a future bet, a parlay bet, a round-robin bet, a handicap bet, an over/under bet, a full cover bet, an accumulator bet, an outright bet, or a teaser bet. In another refinement, the creation, modification, enhancement, or evaluation of the one or more odds occurs dynamically and in real-time or near real-time as new animal data, contextual data, or a combination thereof is gathered by the computing subsystem. In another refinement, the targeted event is comprised of a plurality of targeted events.

In a variation, a method and system for generating dynamic, real-time predictions using animal data (e.g., including, but not limited to, heart rate, heart rate variability, and its derivatives including max heart rate, RMSSD spike count over a specified period of time, and the like) is described. The method and system includes one or more Artificial Intelligence (“AI”)-based models designed and trained with reference animal data (e.g., historical animal data) and reference contextual data (e.g., including reference outcome data), as well as one or more features (i.e., one or more individual measurable properties or characteristics of a phenomenon, which may or may not be numeric in nature) created or modified based on such data (or derived from such data), along with real-time or near-real time animal data gathered and transformed by the system (e.g., which may occur on a single computing device or across multiple computing devices), to generate one or more predictions related to one or more event outcomes. Advantageously, the system is configured to learn from new data entering the system, with such data—and the associated learnings derived from such data—being added to the one or more reference animal databases containing the reference animal data and/or reference contextual data.

In terms of setup, once the animal data is gathered (e.g., collected) by the system for each individual or a group of individuals and the system takes one or more actions to transform the animal data (e.g., normalize, timestamp, aggregate, clean, store, manipulate, denoise, tag, process, enhance, organize, categorize, analyze, anonymize, synthesize, replicate, summarize, productize, synchronize, and the like), the transformed animal data is used to create the one or more features for the one or more AI-based models for each individual or group of individuals. Characteristically, the one or more features created for the one or more AI-based models include real-time or near real-time heart rate (HR) and heart rate variability (e.g., RMSSD, SDNN, LF, HF, LF/HF) measured over n seconds (or any specified time intervals, which is a tunable parameter). In some variations, at least a portion of these animal data metrics can be grouped as short term, medium-term or long term based on the value of time intervals.

Additional metrics can be created or modified such as moving average heart rate over n intervals, rolling time domain HRV features RMSSD and SDNN over n intervals, rolling frequency domain features LF, HF, and LF/HF Ratio over n intervals, and the like (with the one or more intervals being tunable parameters). Cumulative metrics can also be derived; in the context of sports and more specifically analyses by individual player, cumulative metrics can include cumulative mean HR by player per event (e.g., point, game, match) or subset of events (e.g., subset of points or games or matches); cumulative maximum HR by player for each event (e.g., point, game, match) or subset of events (e.g., subset of points or games); and the like. Reference data (e.g., baseline metrics) for each player or group of players derived from the reference animal database (e.g., reference animal data, reference contextual data, or a combination thereof) such as baseline heart rate, baseline RMSSD, and the like (e.g., with reference data including—in some variations—a combination of historical baseline animal data such as historical baseline heart rate, historical baseline RMSSD, and the like and newly-collected animal data also being utilized as a baseline such as newly collected or real-time/near real-time baseline heart rate, baseline RMSSD, and the like, with the newly collected or real-time/near real-time data being collected prior to the start of an event) are then utilized prior to the event (e.g., point, game, match) to derive the difference in values (e.g., delta). The system determines what reference data is being accessed based upon the type of animal data (e.g., the type of one or more metrics) being used. Specific metrics described herein like RMSSD spike count (i.e., SPIKE_RMSSD_COUNT) and/or other HRV-based metrics (e.g., SDNN, LF, HF, LF/HF Ratio, and the like) are then calculated. In some variations, the system may not have access to the reference data (or access the reference data) but instead uses the newly-collected animal data (e.g., the real-time or near real-time animal data gathered by the system or animal data gathered by the system in a period of time leading up to an event) from the individual or group or individuals to create one or more baselines on the fly (i.e., dynamically) that are representative of reference data used for analysis. In another variations, the system may generate artificial data to be used, at least in part, as baseline data (e.g., which can be used in conjunction with animal data derived from the individual or animal data derived from other individuals that may or may not share at least one characteristic with the targeted individual or group of targeted individuals). In a refinement, the system may use one or more lagged values for certain features, where the lags range from 1 to n seconds or time intervals.

In terms of learning, reference data (e.g., reference animal data, reference contextual data) is used as training data for the one or more AI-based models on an individual or group basis. In one variation related to sports where the structure of the sport includes an event hierarchy of point (or the like), game (or the like), and match outcomes (or the like), the training data for each individual player or group of players is organized by events (e.g., points, games, matches) and each of the features is measured in a time period immediately prior to the outcome of the event (e.g., the point, game, match) being determined. In some variations, the time period related to when each of the features is measured can be a tunable parameter. In a refinement, the training data for each individual player or group of players can be organized by a higher tier in the event hierarchy (e.g., game, match, or its equivalent) or a lower tier in the event hierarchy (e.g., an event within a point or its equivalent), or a subset of tiers on the event hierarchy. The reference data is organized such that the system trains the one or more AI models on an initial subset of one or more events (e.g., games played in a match) for each individual player or group of players, which can be a tunable parameter, and tests on one or more later events, which is the holdout set, on a rolling basis. The one or more outcomes (e.g., win/loss) predicted (e.g., point, game, match) for each individual player or group of players based upon the reference data (e.g., test data) are then compared against the actual observed outcomes. Performance of the system can be measured using one or more AI performance measures or indices including, not limited to, confusion matrices, accuracy percentages per event (e.g., point), distribution of mis-classifications over the spectrum of events (e.g., points), and the like. Once the system learns the one or more correlations between the individual's animal data (and in particular its one or more features) and the event outcomes (e.g., win/loss of any particular point, game, match, and the like), the system gathers, transforms, and utilizes the real-time or near real-time animal data to predict the outcome for the next event (e.g., the next point, game, or match). In a refinement, the system can utilize the real-time prediction (e.g., predictive indicator) created, modified, or enhanced for the event—and the associated real-time or near real-time animal data and contextual data (e.g., including event data), as well as insights derived from it—as training data to enhance the prediction accuracy (e.g., enhance the predictive indicator) for one or more future events. In another refinement, the system—leveraging its one or more AI models—may learn one or more correlations between groups of individuals' animal data, the associated contextual data, and one or more associated event outcomes (which the one or more outcomes can be on an individual, group, or group-subset basis), from which the system then utilizes real-time or near real-time animal data gathered from the same group of individuals (or subset of individuals) and transformed by the system to predict one or more outcomes for the next one or more events. In many variations, outcomes are binary such as win/loss in sports. However, in some cases, multiclass outcomes can be modeled (e.g., match abandoned or the player is injured; player runs a certain distance within a certain time).

A variety of AI classification algorithms can be utilized to derive the one or more predictions including Random Forest classification algorithm, Random Forest/Decision Trees, Support Vector Machine classifier, K-Nearest Neighbors, Naive Bayes, Linear Discriminant Analysis, Logistic Regression, Neural Networks, Gradient Boosting Machine Classifier, and the like. However, the invention is not limited to only these types of AI classification algorithms.

In a variation, prediction system 10 can be configured to create, modify, or enhance multiple outcome predictions via a single predictive indicator or multiple predictive indicators. In one variation, the computing subsystem uses a single input variable to create, modify, or enhance one or more outcome predictions via the one or more predictive indicators. In another variation, the computing subsystem uses multiple input variables to create, modify, or enhance one or more outcome predictions via the one or more predictive indicators. In these variations, the outcome can be an end result. In a refinement, the outcome is a definable result based upon one or more tunable parameters that enable the user or system to define the result (e.g., end result). In another refinement, an end result includes a plurality of end results. For example, the outcome may be whether an individual or team wins or loses a game in a sports event. However, the outcome may also be whether the team or individual won the game in under n number of minutes, or whether the team lost the game while shooting above a pre-defined field-goal percentage, or the like. Characteristically, the system can be configured to predict multiple outcomes associated with an event using one or more Artificial Intelligence techniques (e.g., Multi-Class Classification), which can occur simultaneously. For example, the system may be configured to predict an outcome of an event—win/loss of a game for example—along with one or more characteristics (e.g., details) of (or associated with) the event, such as whether the player will win or lose and whether that win or lose will occur in under n number of hours, whether the player win or lose and whether that loss will occur in less than n number of sets, or the like. In another example, the event may be a biological-based event, such as an injury, whereby the system can be configured to predict the likelihood that a player will get injured (or whether a player will get injured) as well as predict one or more details of the event such as the severity or type of injury (e.g., whether the athlete will need to go to the emergency room vs not based upon the injury; whether the athlete will be out for 6-8 weeks, 1 game, or 1 quarter based upon the injury, and the like; whether the athlete will experience back spasms or cramps, tear an ACL, or experience heat stroke, and the like. In this example, the system can also make a further one or more predictions to predict the severity of the heat stroke, the ACL injury, the cramps or back spasms, and the like). In a refinement, a single input variable leads to a single outcome prediction. In another refinement, a single input variable leads to multiple outcome predictions. In another refinement, multiple input variables lead to a single outcome prediction. In another refinement, multiple input variable lead to multiple outcome predictions.

To execute the prediction system, the computing subsystem can be configured to operate in a multi-dimensional space (e.g., spatial and temporal dimensions) with one or more inputs from gathered data that include animal data, its associated contextual data, event data (i.e., which includes event outcome data), or a combination thereof. Characteristically, at least a portion of the one or more inputs is orthogonal data (e.g., environmental data). The dimensionality of the gathered data can be reduced using one or more Artificial Intelligence or Statistical techniques like CNNs (Convolutional Neural Networks), PCA (Principal Component Analysis), or other linear and non-linear dimensionality reduction techniques or processes (or a combination thereof) to identify and extract the most important factors (e.g., derived features or representative features of the data set derived from the one or more inputs) contributing towards a prediction, which could be further used in one or more artificial intelligence techniques to make the one or more real time or near real-time predictions. In a refinement, the dimensionality of the gathered data is reduced using one or more Artificial Intelligence techniques, the one or more techniques including one or more Linear and Non-Linear Dimensionality Reduction techniques or methods, to identify and extract at least one contributing factor (e.g., one transformed or derived feature) towards making a prediction. In another refinement, the one or more predictions are generated using a Multivariate Time Series forecasting technique in a multi-dimensional space (e.g., spatio-temporal predictions) via the use of one or more Classification or Regression techniques. For example, the system could be configured to predict the win/loss over various time horizons or the intensity or magnitude of the win/loss or both. In another example, the system could be configured to predict the physiological state of an individual over various time horizons as well as the associated one or more outcomes in various time horizons. This can be achieved using one or more AI techniques for Multi-class Classification or Multi-Output Regression depending on the type of target variables, the state that is being modelled, or a combination thereof.

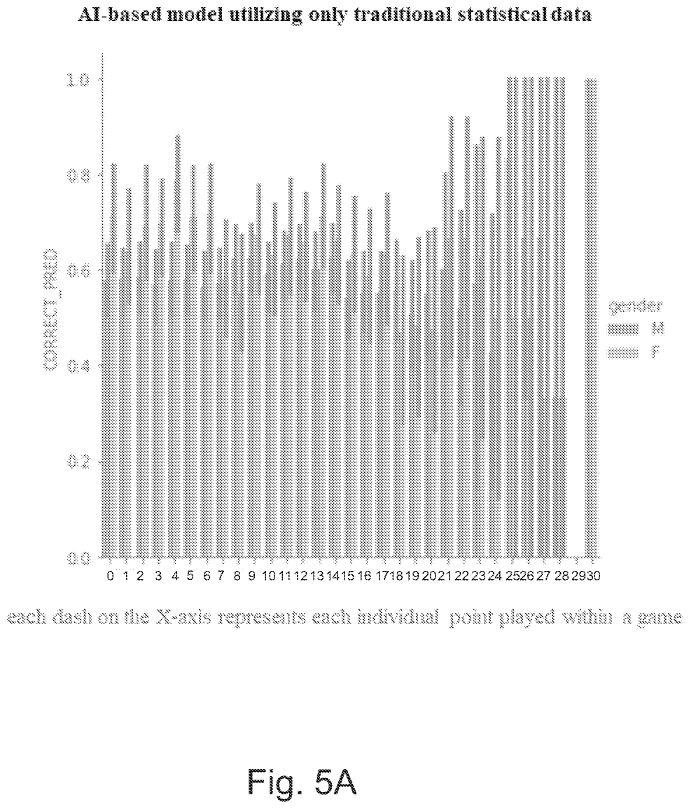

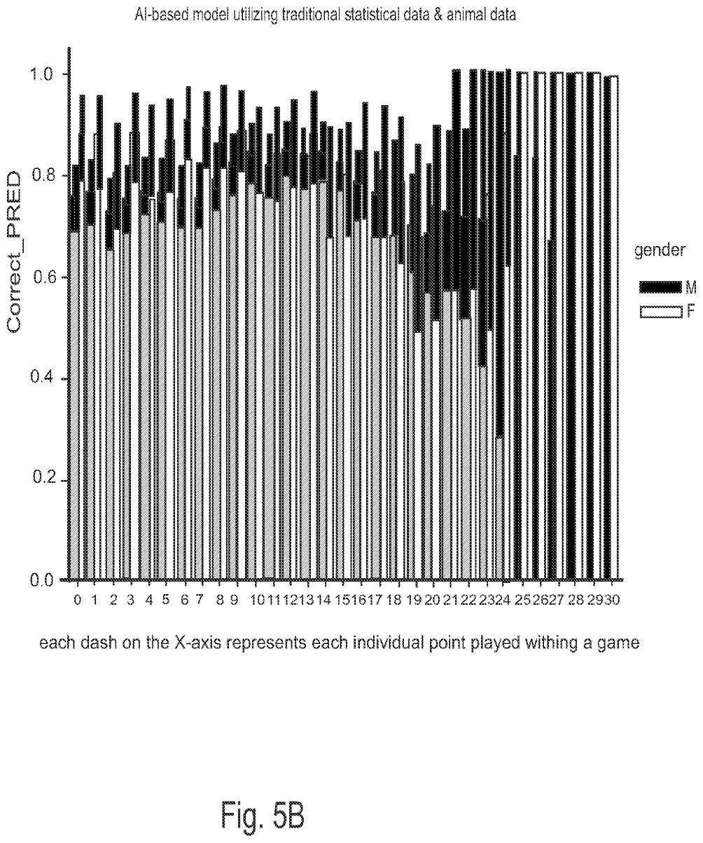

FIGS. 5A and 5B are example illustrations of the output of the AI-based system that utilizes animal data and the techniques described to make one or more predictions. The illustration features the aggregate output of the AI-based model making predictions related to the win/loss of a game in a squash competition at the end of each point played, which includes predictions for both males and females. In 5A and 5B, the x-axis represents each individual point played in a particular game of squash. Note that the model is not limited to squash, individual athlete sports, or sports in general, and can be applied to all sports and non-sports applications. The y-axis represents the number of times (represented as a percentage) the model made the correct win/loss game prediction at the end of each point played across all players (which can also be shown for each individual player of a subset of players).

In FIG. 5A, the model utilizes only traditional statistical data. FIG. 5B utilizes both traditional statistical data (e.g., non-animal data) and animal data including HR and HRV. Utilizing the system and method described, the system is able to generate more accurate predictions related to game win/loss by incorporating animal data and using the techniques described herein compared to using traditional statistics alone.

While exemplary embodiments are described above, it is not intended that these embodiments describe all possible forms of the invention. Rather, the words used in the specification are words of description rather than limitation, and it is understood that various changes can be made without departing from the spirit and scope of the invention. Additionally, the features of various implementing embodiments can be combined to form further embodiments of the invention.

1. A method for generating dynamic real-time predictions using heart rate variability comprising:
gathering or calculating subsequent R-R intervals derived from one or more source sensors from a targeted individual, the subsequent R-R intervals being R-R intervals during an event;
calculating differences in subsequent successive R-R intervals;
calculating one or more subsequent heart rate variability values from the successive differences between heartbeats, wherein one value of the one or more subsequent heart rate variability values is calculated for each sub-event amongst two or more sub-events that comprise at least a portion of the event;
calculating a heart rate variability HRV difference based upon the difference between the heart rate variability values for consecutive sub-events and a heart rate variability baseline, divided by the heart rate variability baseline;
calculating the difference between two successive HRV differences to create at least one variability indicator;
creating a threshold utilizing at least a portion of contextual data to characterize information derived from one or more variability indicators;
creating at least one primary insight by comparing the threshold and the at least one variability indicator;
accessing one or more reference insights; and
comparing the at least one primary insight and the one or more reference insights to create one or more predictive indicators.

2. The method of claim 1 wherein the heart rate variability baseline is determined by:
gathering or calculating R-R intervals derived from the one or more source sensors from a targeted individual prior to the event;
calculating differences in successive R-R intervals;
calculating one or more heart rate variability values from successive differences between heartbeats; heartbeats; and
establishing the heart rate variability baseline for the targeted individual using at least a portion of the calculated one or more heart rate variability values for an event associated with the targeted individual with a definable, quantifiable, measurable, or observable outcome, wherein the heart rate variability baseline is created, modified, or enhanced based upon values collected prior to a start of the event.

3. The method of claim 1, wherein the successive differences between normal heartbeats are calculated by an RMSSD-based methodology or formula.

4. The method of claim 1, wherein the at least one primary insight, the one or more reference insights, or the one or more predictive indicators are modified or enhanced based upon new R-R intervals or new contextual data being calculated or gathered.

5. The method of claim 4, wherein one or more Artificial Intelligence techniques are utilized for one or more modifications or enhancements.

6. The method of claim 1, wherein one or more steps are executed on one or more computing devices, one or more sensors, or a combination thereof.

7. A system for generating dynamic real-time predictions using heart rate variability comprising:
one or more source sensors that gather animal data as gathered animal data from a targeted individual wherein at least a portion of the gathered animal data is heart rate variability data, the animal data being transmitted by the one or more source sensors electronically;
a transmission subsystem that provides transmitted animal data to a computing subsystem; and
a computing subsystem gathers the animal data in real-time or near real-time, wherein the computing subsystem is configured to:
gather contextual data related to the gathered animal data and an event associated with the targeted individual, wherein the computing subsystem is configured to:
take one or more actions with gathered contextual data and the animal data to create, modify, or enhance at least one primary insight related to at least one physiological-based condition of the targeted individual, the computing subsystem being further operable to make one or more modifications or enhancements to the at least one primary insight as additional animal data, additional contextual data, or a combination thereof, is gathered by the computing subsystem; and
access at least one reference insight, the at least one reference insight and the at least one primary insight being used to create, modify, or enhance at least one predictive indicator, and wherein the at least one predictive indicator is used by one or more computing devices to perform one or more of: (1) create, modify, enhance, or evaluate one or more odds; (2) create, modify, enhance, or evaluate one or more bets; (3) as a market upon which one or more bets are placed or accepted; (4) formulate one or more strategies; (5) create, modify, enhance, acquire, offer, or distribute one or more products; and/or (6) mitigate, prevent, or take one or more risks.

8. The system of claim 7, wherein the at least one reference insight, the at least one primary insight, the at least one predictive indicator, or combinations thereof, are used as training data for one or more Artificial Intelligence techniques to create, modify, or enhance one or more new predictive indicators.

9. The system of claim 8, wherein the computing subsystem is configured to execute one or more steps of:
gathering or calculating subsequent R-R intervals derived from one or more source sensors from a targeted individual, the subsequent R-R intervals being R-R intervals during an event;
calculating differences in subsequent successive R-R intervals;
calculating one or more subsequent heart rate variability values from the successive differences between heartbeats, wherein one value of the one or more subsequent heart rate variability values is calculated for each sub-event amongst two or more sub-events that comprise at least a portion of the event;
calculating a heart rate variability HRV difference based upon the difference between the heart rate variability values for consecutive sub-events and a heart rate variability baseline, divided by the heart rate variability baseline;
calculating the difference between two successive HRV differences to create at least one variability indicator;
creating a threshold utilizing at least a portion of contextual data to characterize information derived from one or more variability indicators;
creating at least one primary insight by comparing the threshold and the at least one variability indicator;
accessing one or more reference insights; and
comparing the at least one primary insight and the one or more reference insights to create one or more predictive indicators.

10. The system of claim 9, wherein the one or more bets include at least one of: a proposition bet, spread bet, a line bet, a future bet, a parlay bet, a round-robin bet, a handicap bet, an over/under bet, a full cover bet, an accumulator bet, an outright bet, or a teaser bet.

11. A system for generating dynamic real-time predictions using heart rate variability comprising:
one or more source sensors that gather animal data from a targeted individual in real-time or near real-time prior to or during a targeted event, wherein at least a portion of the gathered animal data is heart rate variability data, the animal data being transmitted by the one or more source sensors electronically;
a transmission subsystem that provides transmitted animal data to a computing subsystem; and
a computing subsystem that gathers the animal data and associated contextual data related to the gathered animal data, the associated contextual data including event data from the targeted event associated with the targeted individual, wherein the computing subsystem is configured to:
take one or more actions to transform the gathered animal data and the associated contextual data as transformed data, including the event data from the targeted event, into a data format wherein at least a portion of the transformed data is used to create, modify, or enhance one or more features, the one or more features including information derived from heart rate variability data measured over an adjustable time interval, and wherein each of the features is measured in an adjustable time period prior to an outcome of the targeted event being determined;
take one or more actions to transform the gathered animal data and associated contextual data into reference data, wherein at least a portion of the reference data is used to create, modify, or enhance one or more features, the one or more features including information derived from heart rate variability data measured over an adjustable time interval, and wherein each of the one or more features is measured in an adjustable time period prior to an outcome associated with the event being determined;
access one or more baselines for the targeted individual prior to each targeted event, the computing subsystem utilizing the one or more baselines and the real-time or near real-time animal data or its one or more derivatives to perform one or more calculations to derive a difference in values; and
compare the difference in values to create, modify, or enhance at least one predictive indicator related to the outcome of the targeted event or another targeted event, and wherein the at least one predictive indicator is used by one or more computing devices to at least one of: (1) create, modify, enhance, or evaluate one or more odds; (2) create, modify, enhance, or evaluate one or more bets; (3) as a market upon which one or more bets are placed or accepted; (4) formulate one or more strategies; (5) create, modify, enhance, acquire, offer, or distribute one or more products; or (6) mitigate, prevent, or take one or more risks.

12. The system of claim 11, wherein the computing subsystem is configured to execute one or more steps to create, modify, or enhance the at least one predictive indicator in the real-time or near real-time.

13. The system of claim 11, wherein the computing subsystem is configured to execute one or more steps of:
gathering or calculating R-R intervals derived from one or more source sensors from a targeted individual;
calculating differences in successive R-R intervals;
calculating one or more heart rate variability values from successive differences between normal heartbeats;
establishing a heart rate variability baseline for the targeted individual using at least a portion of the calculated one or more heart rate variability values for an event associated with the targeted individual with a definable, quantifiable, measurable, or observable outcome, wherein the heart rate variability baseline is created, modified, or enhanced based upon values collected prior to a start of the event;
gathering or calculating subsequent R-R intervals derived from the one or more source sensors from the targeted individual;
calculating differences in subsequent successive R-R intervals;
calculating one or more subsequent heart rate variability values from the successive differences between normal heartbeats, wherein one value of the one or more subsequent heart rate variability values is calculated for each sub-event amongst two or more sub-events that comprise at least a portion of the event;
calculating a HRV difference based upon the difference between the heart rate variability values for consecutive sub-events and the heart rate variability baseline, divided by the heart rate variability baseline;
calculating the difference between two successive HRV differences to create at least one variability indicator;
creating a threshold utilizing at least a portion of contextual data to characterize information derived from one or more variability indicators;
creating at least one primary insight by comparing the threshold and the at least one variability indicator;
accessing one or more reference insights; and
comparing the at least one primary insight and the one or more reference insights to create one or more predictive indicators.

14. The system of claim 11, wherein the computing subsystem is configured to execute one or more steps for a plurality of targeted individuals, either collectively or individually, to create, modify, or enhance at least one predictive indicator in the real-time or near real-time.

15. The system of claim 11, wherein the computing subsystem gathers the reference data directly or indirectly associated with the targeted individual and/or the targeted event from one or more other computing devices.

16. The system of claim 11, wherein the computing subsystem does not have access to reference data but instead uses newly-gathered animal data from the targeted individual to create the one or more baselines that are representative of reference data and used for analysis.

17. The system of claim 11, wherein the computing subsystem uses one or more lagged values for one or more select features, wherein the one or more lagged values range from 1 to any number of seconds or time intervals.

18. The system of claim 11, wherein the computing subsystem is further operable to calculate one or more additional metrics, the one or more additional metrics including SPIKE_RMSSD_COUNT.

19. The system of claim 11, wherein performances of wherein one or more Artificial Intelligence-based models are measured by using one or more AI performance measures or indices which include at least one of: confusion matrices, accuracy percentages per event, or distribution of mis-classifications over a spectrum of events.

20. The system of claim 19, wherein the reference data used to design and train the one or more Artificial Intelligence-based models for each targeted individual or group of targeted individuals is organized by a higher tier in an event hierarchy, a lower tier in the event hierarchy, or a subset of tiers on the event hierarchy.

21. The system of claim 11, wherein the computing subsystem is trained to learn one or more correlations between animal data gathered from a plurality of individuals and the associated contextual data from the plurality of individuals, the associated contextual data including one or more associated event outcomes.

22. The system of claim 21, wherein the computing subsystem utilizes the real-time or the near real-time animal data, or its one or more derivatives, and the associated contextual data gathered from the targeted individual or a group of targeted individuals, a group of targeted individuals being sourced from the plurality of individuals, to create, modify, or enhance the at least one predictive indicator for the targeted individual or a group of targeted individuals related to one or more outcomes of the targeted event or another one or more events.

23. The system of claim 22, wherein the one or more outcomes of the targeted event is a binary outcome.

24. The system of claim 22, wherein the one or more outcomes of the targeted event is a multiclass outcome.

25. The system of claim 11, wherein the at least one predictive indicator is derived utilizing at least one classification algorithm selected from the group consisting of Random Forest classification algorithm, Random Forest/Decision Trees, Support Vector Machine classifier, K-Nearest Neighbors, Naive Bayes, Linear Discriminant Analysis, Logistic Regression, Neural Networks, and Gradient Boosting Machine Classifier.

26. The system of claim 11, wherein the one or more features include at least one of: moving average heart rate over an adjustable time interval, rolling time domain HRV features RMSSD and SDNN over an adjustable time interval, rolling frequency domain features LF, HF, and LF/HF Ratio over an adjustable time interval, or one or more cumulative metrics.

27. The system of claim 11, wherein the one or more bets include at least one of: a proposition bet, spread bet, a line bet, a future bet, a parlay bet, a round-robin bet, a handicap bet, an over/under bet, a full cover bet, an accumulator bet, an outright bet, or a teaser bet.

28. The system of claim 11, wherein creation, modification, enhancement, or evaluation of the one or more odds occurs dynamically and in real-time or near real-time as new animal data, contextual data, or a combination thereof is gathered by the computing subsystem.

29. The system of claim 11, wherein the one or more features includes information derived from heart rate data.

30. The system of claim 11, wherein the computing subsystem is trained with reference data from one or more other individuals, at least a portion of the reference data from the one or more other individuals being utilized to create, modify, or enhance the at least one predictive indicator related to the targeted individual.

31. The system of claim 11, wherein the targeted event is comprised of a plurality of targeted events.

32. The system of claim 11, wherein the computing subsystem uses a single input variable to create, modify, or enhance one or more outcome predictions via the one or more predictive indicators.

33. The system of claim 11, wherein the computing subsystem uses multiple input variables to create, modify, or enhance one or more outcome predictions via the one or more predictive indicators.

34. The system of claim 11, wherein the computing subsystem is configured to operate in a multi-dimensional space with one or more inputs from gathered data that include animal data, its associated contextual data, event outcome data, or a combination thereof, with at least a portion of the one or more inputs being orthogonal data.

35. The system of claim 34, wherein the dimensionality of the gathered data is reduced using one or more Artificial Intelligence techniques, the one or more techniques including one or more Linear and Non-Linear Dimensionality Reduction techniques or methods, to identify and extract at least one contributing factor towards making a prediction.

36. The system of claim 34, wherein one or more predictions are generated using a Multivariate Time Series forecasting technique in a multi-dimensional space via the use of one or more Classification or Regression techniques.

37. A system for generating dynamic real-time predictions using heart rate variability comprising:
one or more sensors that gather as gathered animal data from a targeted individual associated with an event wherein at least a portion of the gathered animal data is heart rate variability;
animal data that is transmitted by the one or more sensors electronically;
a transmission subsystem that provides transmitted animal data to a computing subsystem; and
a computing subsystem that gathers the animal data and contextual data associated with the gathered animal data, the contextual data including event data associated with the targeted individual, wherein the computing subsystem is configured to:
take one or more actions to transform the gathered animal data and contextual data into reference data, wherein at least a portion of the reference data is used to create, modify, or enhance one or more features, the one or more features including information derived from heart rate variability data measured over an adjustable time interval, and wherein each of the one or more features is measured in an adjustable time period prior to an outcome associated with the event being determined;
organize the reference data for the targeted individual by event such that the computing subsystem implements one or more Artificial Intelligence-based models designed and trained with the reference data, the reference data including the one or more features and event data, on an initial subset of one or more events associated with the targeted individual from which a predictive indicator for each event outcome is generated and compared against actual outcomes, wherein the one or more Artificial Intelligence-based models are further tested on a holdout data set derived from at least a portion of the event data on a rolling basis to validate accuracy of one or more model performances; and
correlate one or more aspects of a targeted individual's reference data, the one or more aspects including the one or more features and the event data, to create one or more baselines for the targeted individual.

38. A method for generating dynamic real-time predictions using heart rate variability comprising:
gathering or calculating R-R intervals derived from one or more source sensors from a targeted individual;
calculating differences in successive R-R intervals;
calculating one or more heart rate variability values from successive differences between normal heartbeats;
establishing a heart rate variability baseline for the targeted individual using at least a portion of the calculated one or more heart rate variability values for an event associated with the targeted individual with a definable, quantifiable, measurable, or observable outcome, wherein the heart rate variability baseline is created, modified, or enhanced based upon values collected prior to a start of the event;
gathering or calculating subsequent R-R intervals derived from the one or more source sensors from the targeted individual;
calculating differences in subsequent successive R-R intervals;
calculating one or more subsequent heart rate variability values from the successive differences between normal heartbeats, wherein one value of the one or more subsequent heart rate variability values is calculated for each sub-event amongst two or more sub-events that comprise at least a portion of the event;
calculating a HRV difference based upon the difference between the heart rate variability values for consecutive sub-events and the heart rate variability baseline, divided by the heart rate variability baseline;
calculating the difference between two successive HRV differences to create at least one variability indicator;
creating a threshold utilizing at least a portion of contextual data to characterize information derived from one or more variability indicators;
creating at least one primary insight by comparing the threshold and the at least one variability indicator,
accessing one or more reference insights; and
comparing the at least one primary insight and the one or more reference insights to create one or more predictive indicators.
